(19) United States
(12) Patent Application Publication
(10) Pub. No.: US 2009/0181386 A1
(43) Pub. Date: Jul. 16, 2009

(54) Calving Characteristics

(75) Inventors: Mogens Sandø Lund, Ørum (DK); Christian Bendixen, Ulstrup (DK); Helle Jensen, Viborg (DK); Bo Thomsen, &Aring;rhus (DK); Peter Sørensen, Viborg (DK); Søren Svendsen, Randers (DK); Vivi Hunnicke Nielsen, Tjele (DK); Bente Flügel Majgren, Hobro (DK); Bernt Guldbrandtsen, &Aring;rhus (DK); Jørn Rind Thomasen, Holstebro (DK)

Correspondence Address: WEINGARTEN, SCHURGIN, GAGNEBIN & LEBOVICI LLP, TEN POST OFFICE SQUARE, BOSTON, MA, 02109, US

(73) Assignees: KVAEGAVLSFORENINGEN DANSIRE, Randers (DK); AARHUS UNIVERSITET, Arhus C (DK)

(21) Appl. No.: 12/223,773

(22) PCT Filed: Feb. 5, 2007
(86) PCT No.: PCT/DK2007/000060

§ 371 Date: Nov. 6, 2008

(30) Foreign Application Priority Data
Feb. 8, 2006 (DK) PA 2006 00181
Jan. 31, 2007 (DK) PA 2007 00165

(51) Int. Cl. C12Q 1/68

(52) U.S. Cl. 435/6

(57) ABSTRACT

The invention relates to a method for determining calving characteristics in bovine subjects, wherein calving characteristics comprise stillbirth, calving difficulty and calf size at birth, which are all economically important factors. In particular, the method of the invention involves identification of genetic markers and/or Quantitative Trait Locus (QTL) for the determination of calving characteristics in a bovine subject. The determination of calving characteristics involves resolution of the specific microsatellite status. Furthermore, the invention relates to a diagnostic kit for detection of genetic marker(s) associated with calving characteristics. The method and kit of the present invention can be applied for selection of bovine subjects for breeding purposes. Thus, the invention provides a method of genetically selecting bovine subjects with calving characteristics that will yield cows less prone to stillbirth, calving difficulties and undesired calf size at birth.

### FIELD OF INVENTION

The present invention relates to calving characteristics in bovine subjects. In particular, the invention relates to genetic markers for the determination of calving characteristics in a bovine subject and a diagnostic kit for detection of genetic marker(s) associated with calving characteristics.

### BACKGROUND OF INVENTION

Stillbirth, calving difficulty and calf size at birth are economic important calving traits, which are included in the Danish dairy cattle breeding program (Pedersen et al., 2003). The incidence of stillbirths for Holstein cattle has increased in several Holstein populations during the last two decades (Hansen et al., 2004). The increased incidence of stillbirths reduces the potential number of replacement heifers in dairy cattle herds and is associated with ethical problems.

Both direct and maternal genetic components are associated with the calving traits. In Danish Holstein, the heritability (h²) estimates of the calving traits, measured as a direct sire effect (h²=0.05-0.19) are higher than the heritability estimates of the calving traits measured as a maternal grand sire effect (h²=0.04-0.06). The genetic correlation between calving traits measured as a direct sire effect (0.69-0.93) are markedly higher than the genetic correlation between calving traits measured as a maternal grand sire effect (0.01-0.62). The genetic component associated with the calving traits may be due to the segregation of one or more quantitative trait loci (QTL).

Quantitative trait locus (QTL) is a region of DNA that is associated with a particular trait (e.g. a disease or calving characteristics). A QTL is not necessarily a gene itself, but rather a DNA region that is closely linked to the genes that underlie the trait in question. Most likely, a QTL is a set of genes that collectively encode a quantitative trait that varies continuously across a population. Thus, the allelic variation of the QTL is associated with variation in a quantitative trait. The presence of QTL is inferred from genetic mapping, in which the genetic location of the QTL is determined relative to known genetic markers.

The identification of genetic markers that are linked to a particular phenotype, such as calving traits or to a heritable disease, has been facilitated by the discovery of microsatellite markers as a source of polymorphic markers and single nucleotide polymorphisms linked to a mutation causing a specific phenotype. Markers linked to the mutation or the mutation itself causing a specific phenotype of interest are localised by use of genetic analysis in pedigrees and also by exploiting linkage disequilibrium (LD) when looking at populations

Linkage disequilibrium reflects recombination events dating back in history and the use of LD mapping within families increases the resolution of mapping. LD exists when observed haplotypes in a population do not agree with the haplotype frequencies predicted by multiplying together the frequency of individual genetic markers in each haplotype. In this respect the term haplotype means a set of closely linked genetic markers present on one chromosome which tend to be inherited together.

In order for LD mapping to be efficient the density of genetic markers needs to be compatible with the distance across which LD extends in the given population. In a study of LD in dairy cattle population using a high number of genetic markers (284 autosomal microsatellite markers) it was demonstrated that LD extends over several tens of centimorgans for intrachromosomal markers (Farnir et al. 2000). Similarly, Georges, M (2000) reported that the location of a genetic marker that is linked to a particular phenotype in livestock typically has a confidence interval of 20-30 cM (corresponding to maybe 500-1000 genes) (Georges, M., 2000). The existence of linkage disequilibrium is taken into account in order to use maps of particular regions of interest with high confidence.

Several QTL have been detected for calving traits in other Holstein populations (e.g. Kühn et al., 2003; Schrooten et al., 2000; Elo et al., 1999). Some QTL may affect more than one trait, and some QTL may even be located at the same chromosomal position for different traits. If the QTL affects multiple traits then it is important, for e.g. selection purposes, to test if it is a pleiotropic or linked QTL affecting the traits.

Calving traits such as stillbirth, calving difficulty and calf size are not easily predicted. The use of genetic analysis and genetic selection appears to be a possible method for prognostication of these calving traits. Once mapped, a QTL can be usefully applied in marker assisted selection.

### SUMMARY OF INVENTION

It is an object of the present invention to provide an application method for marker assisted selection of polymorphisms in the bovine genome, wherein polymorphisms are associated with calving trait characteristics, such as still birth, calving difficulties, and calf size; and/or provide genetic markers for use in such a method, and/or to provide animals selected using the method of the invention.

One aspect of the present invention relates to a method of determining calving characteristics in a bovine subject, comprising detecting in a sample from said bovine subject the presence or absence of at least one genetic marker that is linked to at least one trait indicative of increased risk of stillbirth and/or increased risk of calving difficulties and/or increased risk of non-desired calf size, wherein said at least one genetic marker is located on the bovine chromosome BTA3 in a region flanked by and including polymorphic microsatellite markers INRA006 and BM7225 and/or
BTA4 in the region flanked by and including polymorphic microsatellite markers BMS1788 and MGTG4B and/or,
BTA5 in the region flanked by and including polymorphic microsatellite markers BMS1095 and BM2830 and/or,
BTA7 in a region flanked by and including polymorphic microsatellite markers BM7160 and BL1043 and/or,
BTA8 in a region flanked by and including polymorphic microsatellite markers IDVGA-11 and BMS836 and/or,
BTA9 in a region flanked by and including polymorphic microsatellite markers BMS2151 and BMS1967 and/or,
BTA10 in a region flanked by and including polymorphic microsatellite markers DIK2658 and BMS2614 and/or,
BTA11 in the region flanked by and including polymorphic microsatellite markers BM716 and HEL13 and/or,
BTA12 in a region flanked by and including polymorphic microsatellite markers BMS410 and BMS2724 and/or,
BTA15 in a region flanked by and including polymorphic microsatellite markers BR3510 and BMS429 and/or,
BTA18 in a region flanked by and including polymorphic microsatellite markers IDVGA-31 and DIK4013 and/or,
BTA19 in a region flanked by and including polymorphic microsatellite markers BM9202 and BMS601 and/or,
BTA20 in a region flanked by and including polymorphic microsatellite markers BM3517 and UWCA26 and/or,
BTA21 in a region flanked by and including polymorphic microsatellite markers DIK5182 and IDVGA-30 and/or,
BTA22 in a region flanked by and including polymorphic microsatellite markers CSSM26 and BM4102 and/or,
BTA24 in a region flanked by and including polymorphic microsatellite markers BMS917 and BMS3024 and/or,
BTA25 in a region flanked by and including polymorphic microsatellite markers ILSTS102 and AF5 and/or,
BTA26 in a region flanked by and including polymorphic microsatellite markers BMS651 and BM7237 and/or,
BTA28 in a region flanked by and including polymorphic microsatellite markers,
BMC6020 and BMC2208, wherein the presence of said at least one genetic marker is indicative of calving characteristics of said bovine subject and/or off-spring therefrom.

A second aspect of the present invention relates to diagnostic kit for use in detecting the presence in a bovine subject of at least one genetic marker associated with bovine calving characteristics, comprising at least one oligonucleotide sequence, wherein the nucleotide sequences are selected from any of SEQ ID NO.: 1 to SEQ ID NO.: 558 and/or any combination thereof.

### DESCRIPTION OF DRAWINGS

FIG. 1: Genome scan of BTA3 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

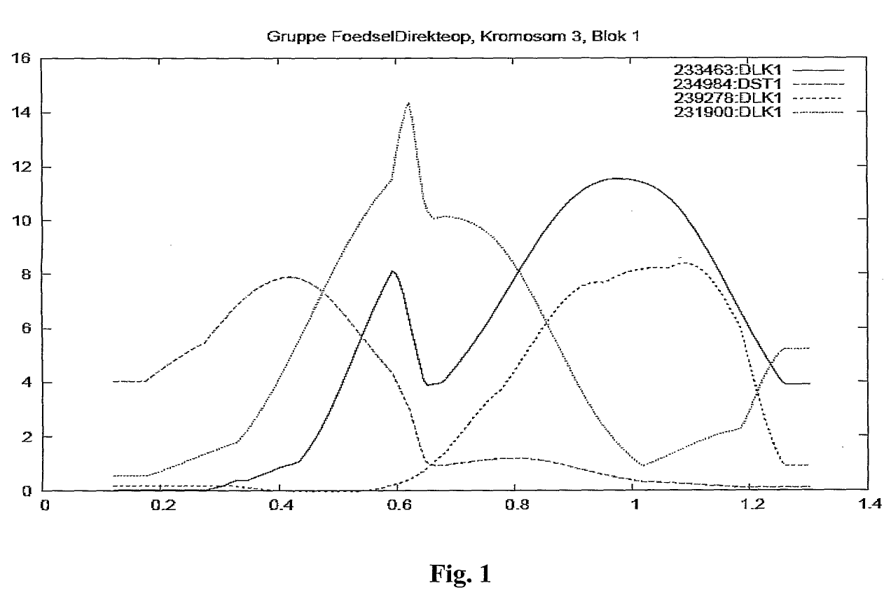

FIG. 2: Genome scan of BTA4 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

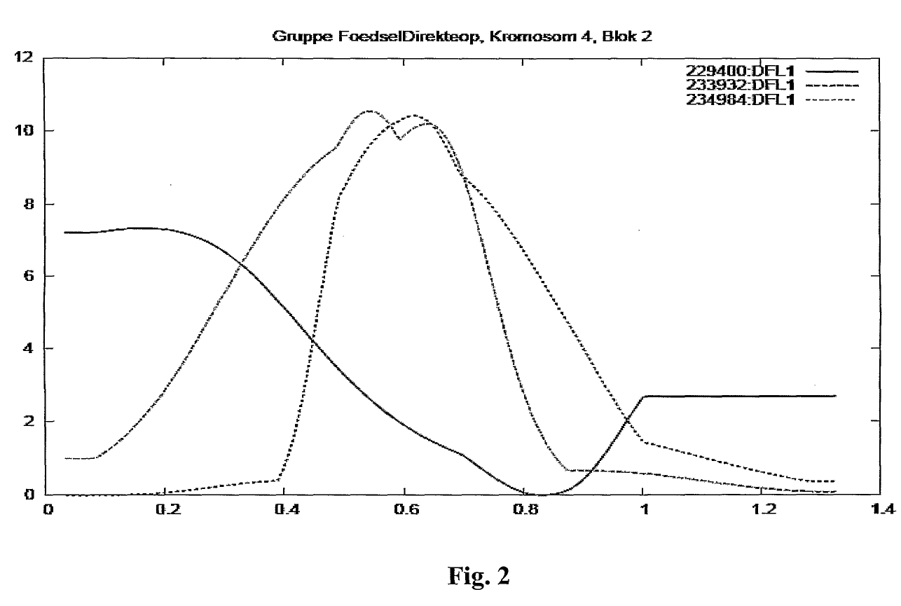

FIG. 3: Genome scan of BTA7 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

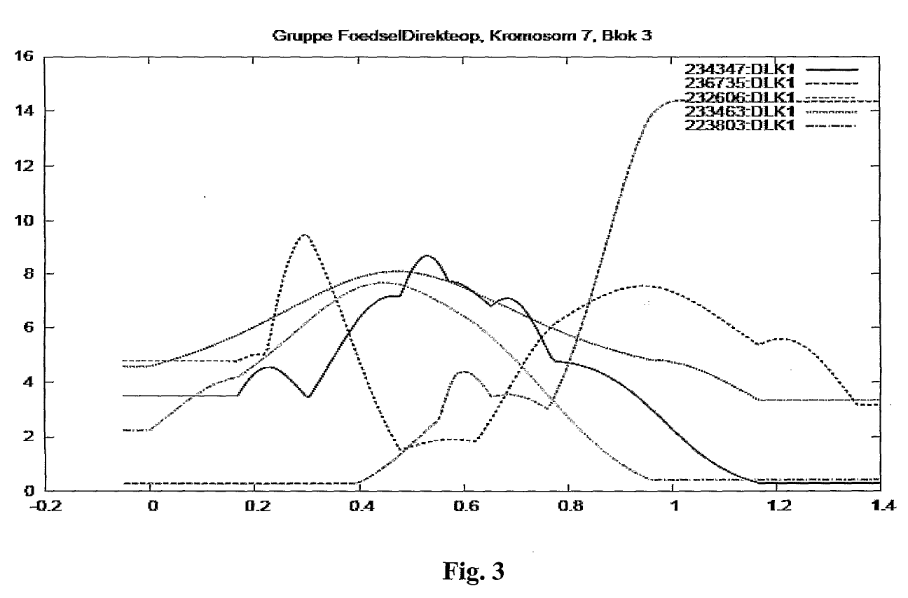

FIG. 4: Genome scan of BTA7 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

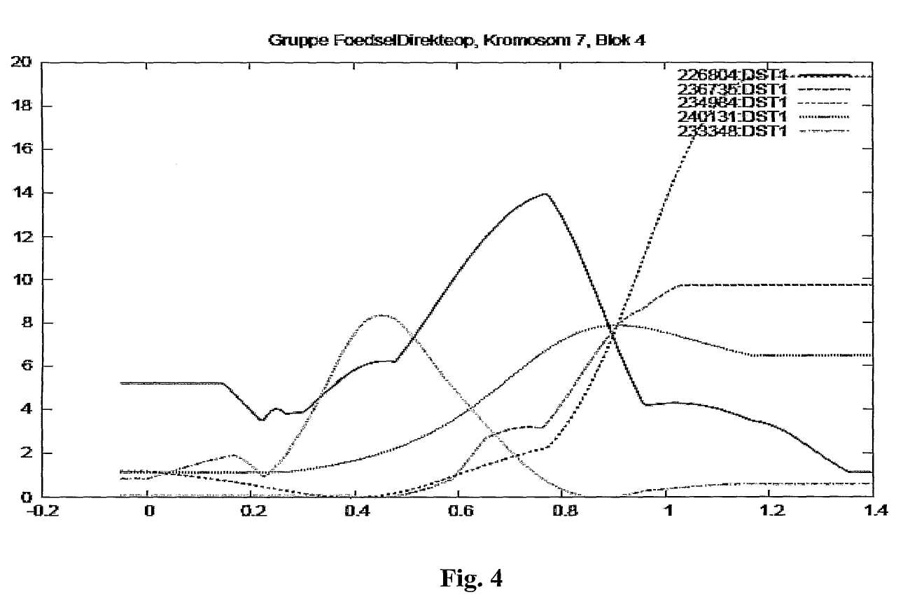

FIG. 5: Genome scan of BTA8 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

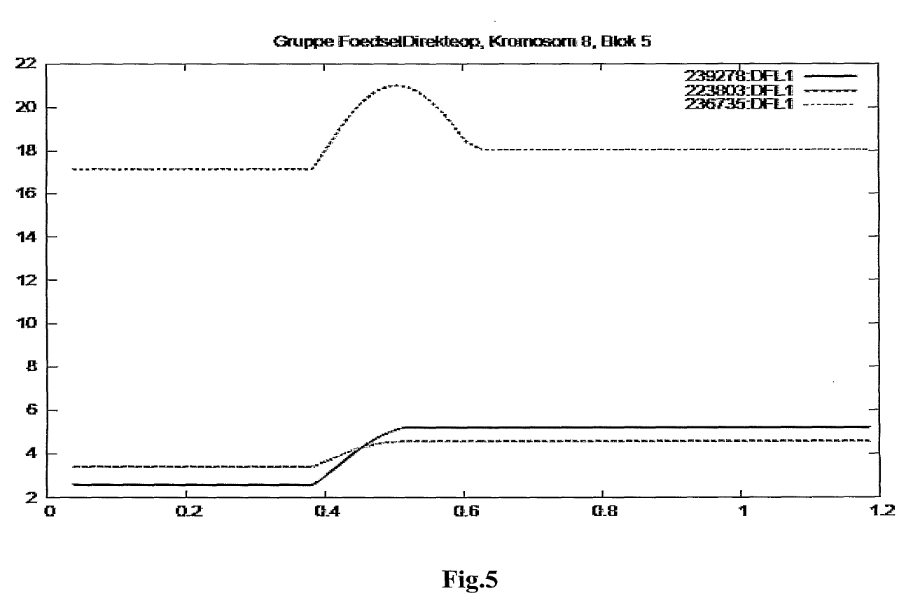

FIG. 6: Genome scan of BTA8 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number I in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

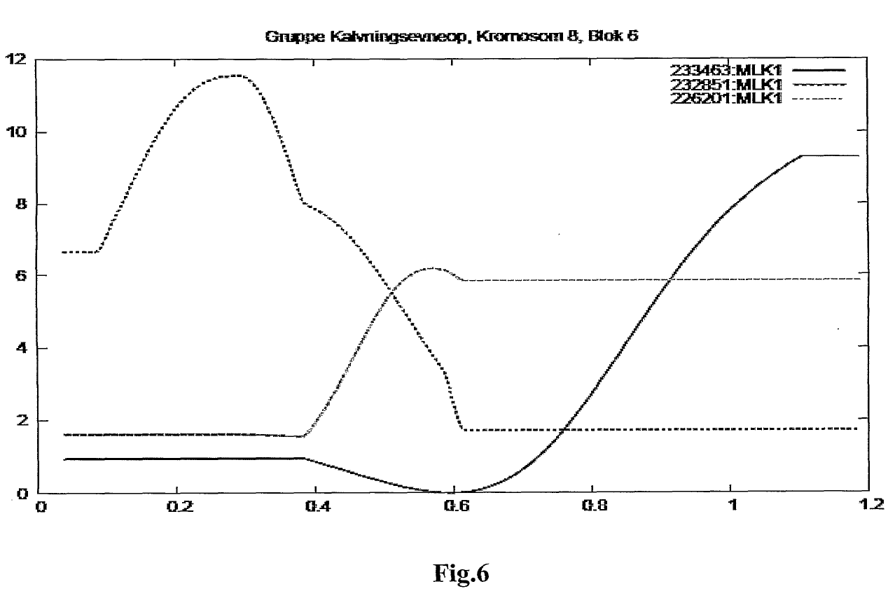

FIG. 7: Genome scan of BTA9 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

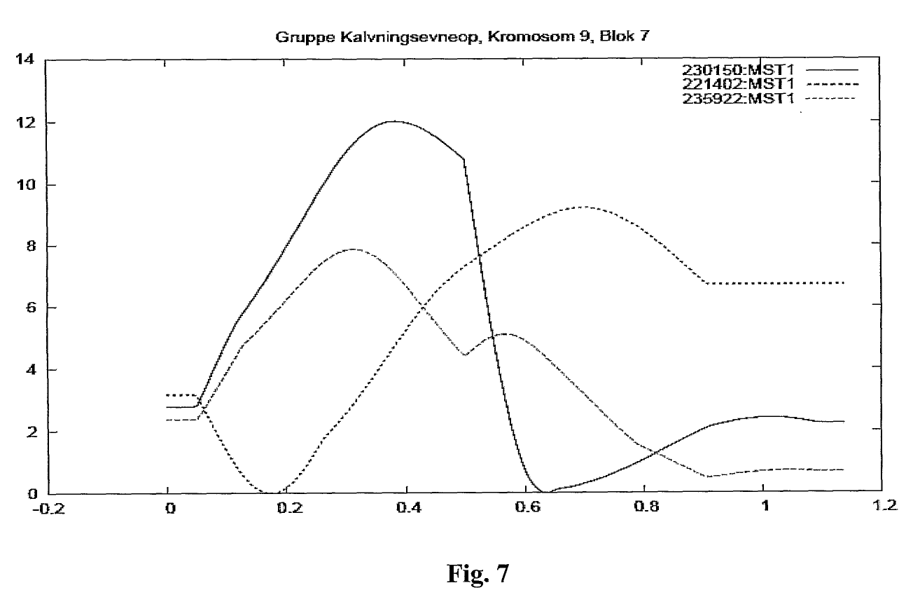

FIG. 8: Genome scan of BTA10 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

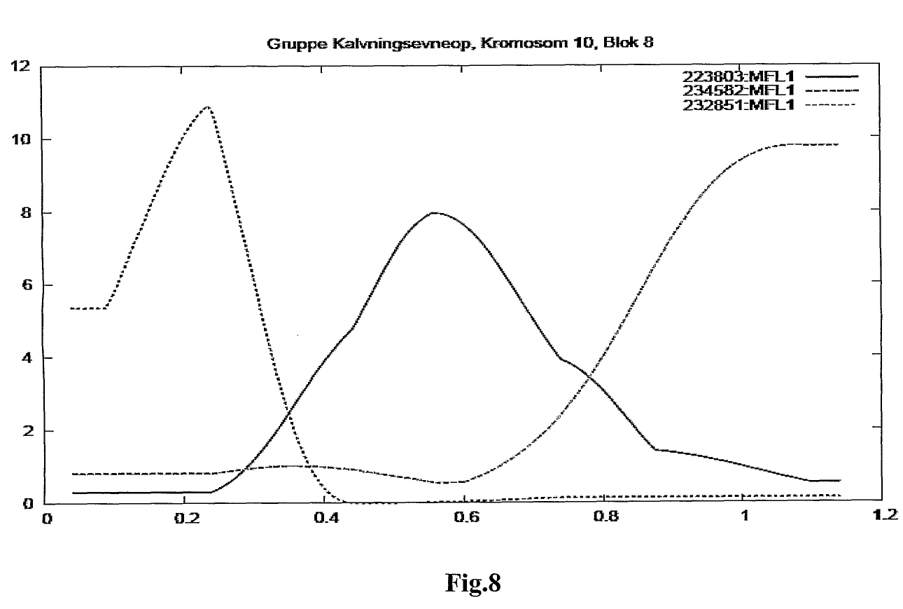

FIG. 9: Genome scan of BTA12 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

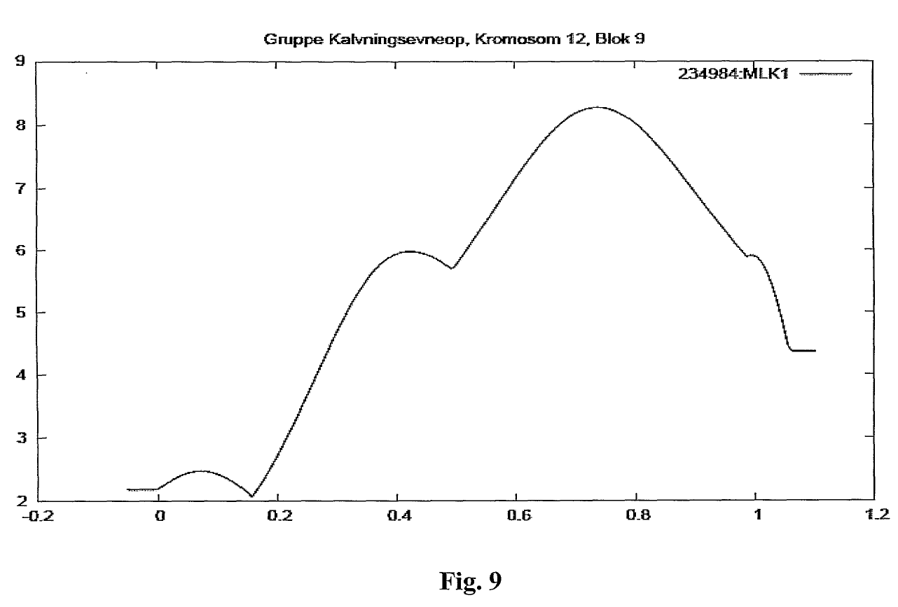

FIG. 10: Genome scan of BTA12 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

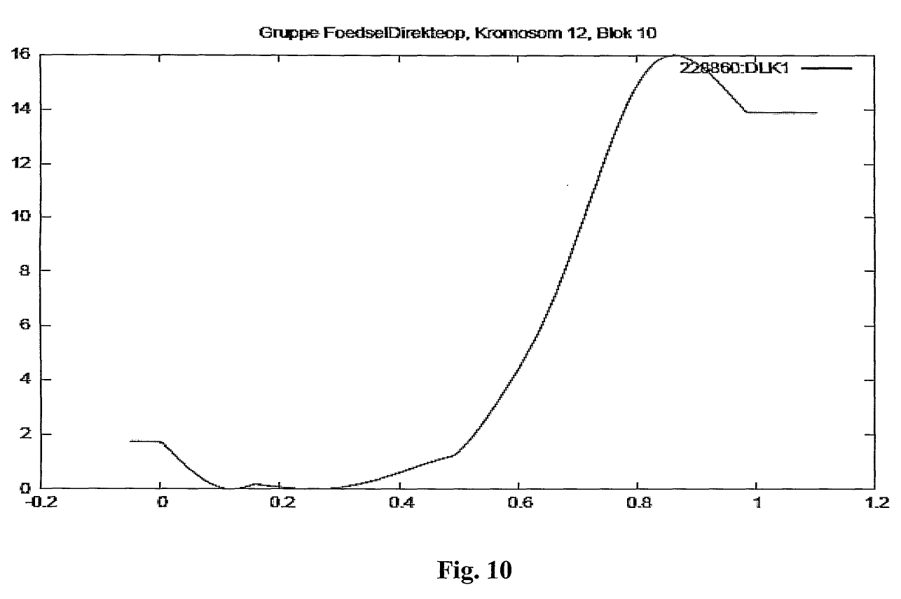

FIG. 11: Genome scan of BTA15 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

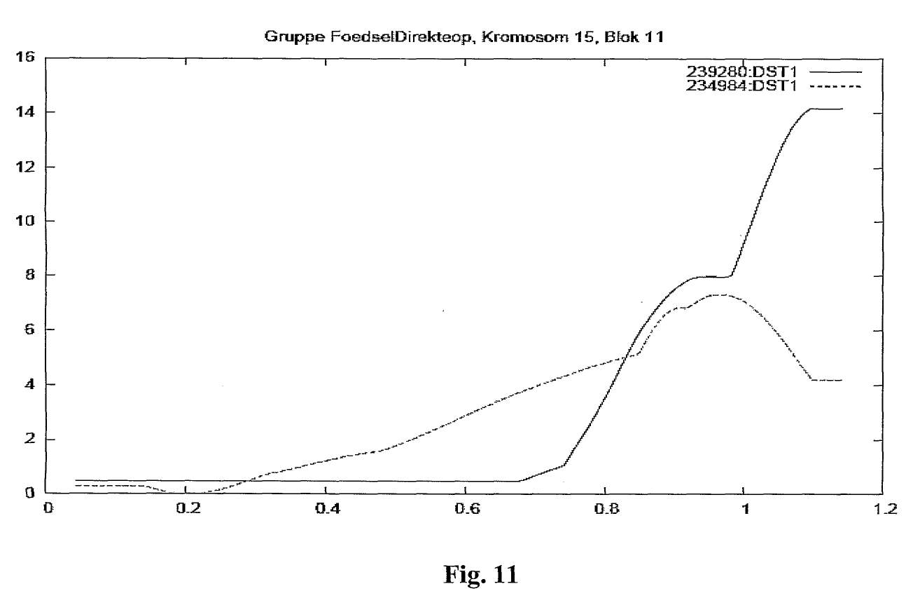

FIG. 12: Genome scan of BTA18 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

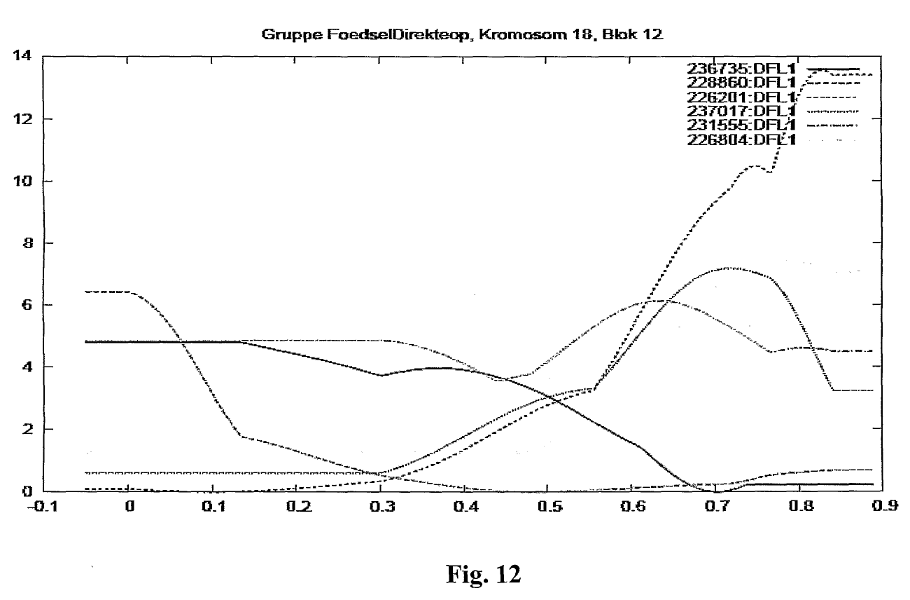

FIG. 13: Genome scan of BTA18 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

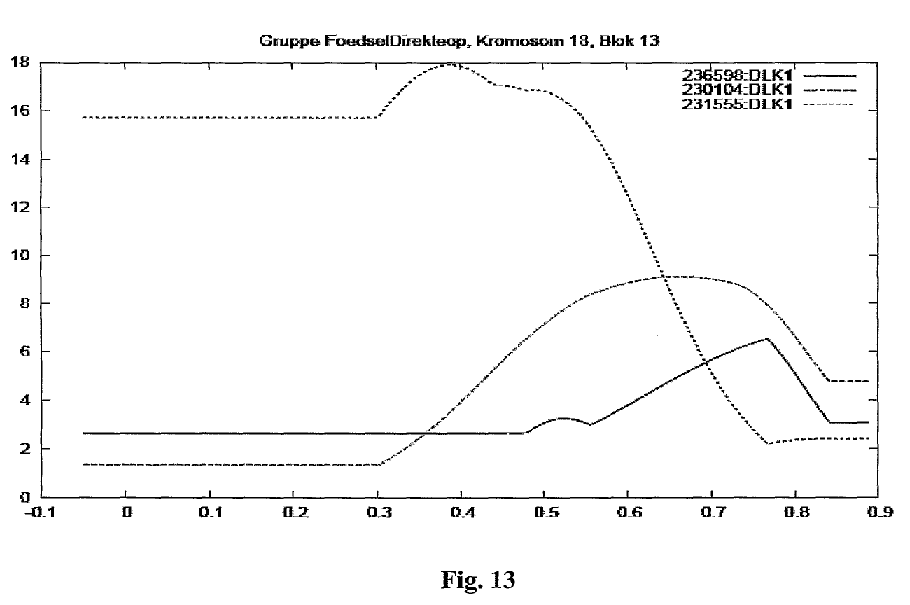

FIG. 14: Genome scan of BTA18 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

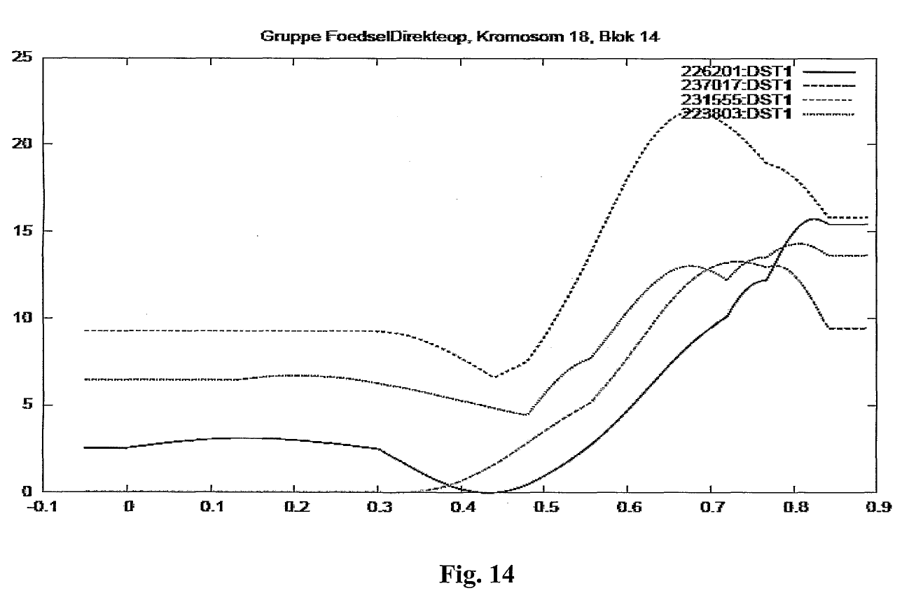

FIG. 15: Genome scan of BTA18 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

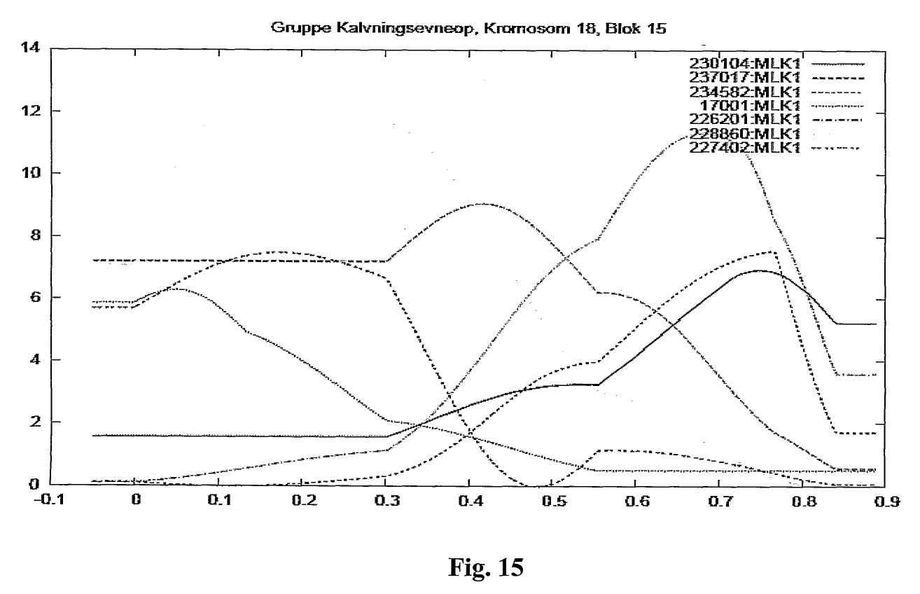

FIG. 16: Genome scan of BTA19 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

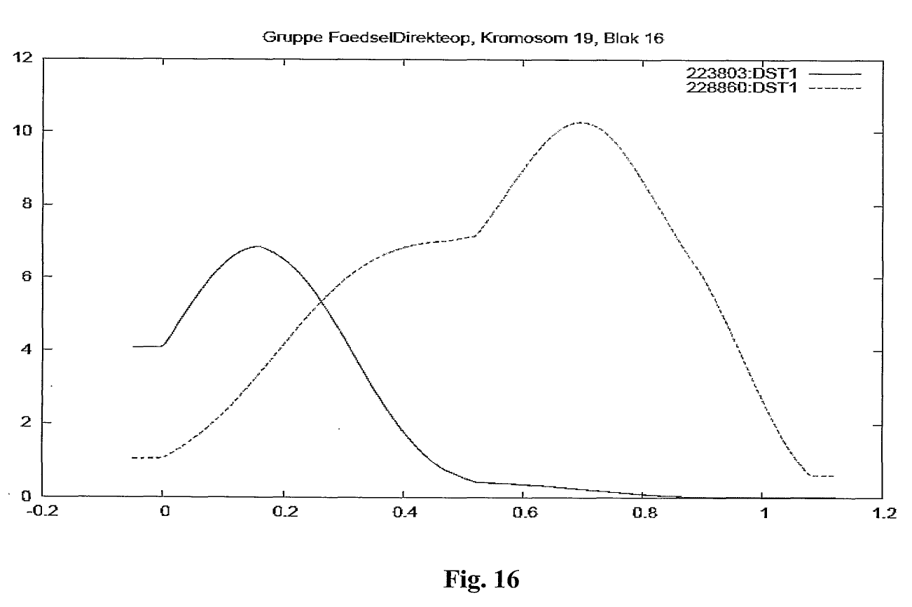

FIG. 17: Genome scan of BTA20 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

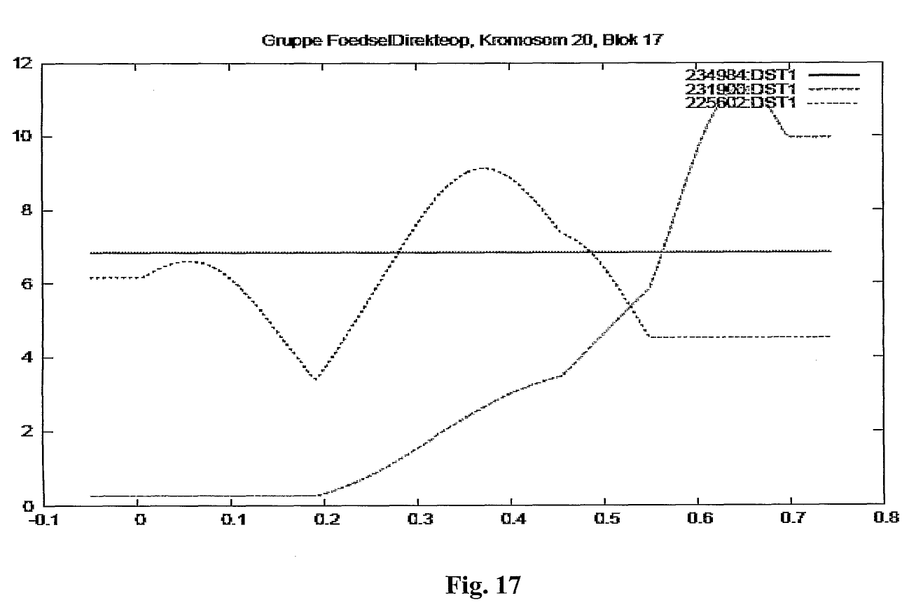

FIG. 18: Genome scan of BTA21 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

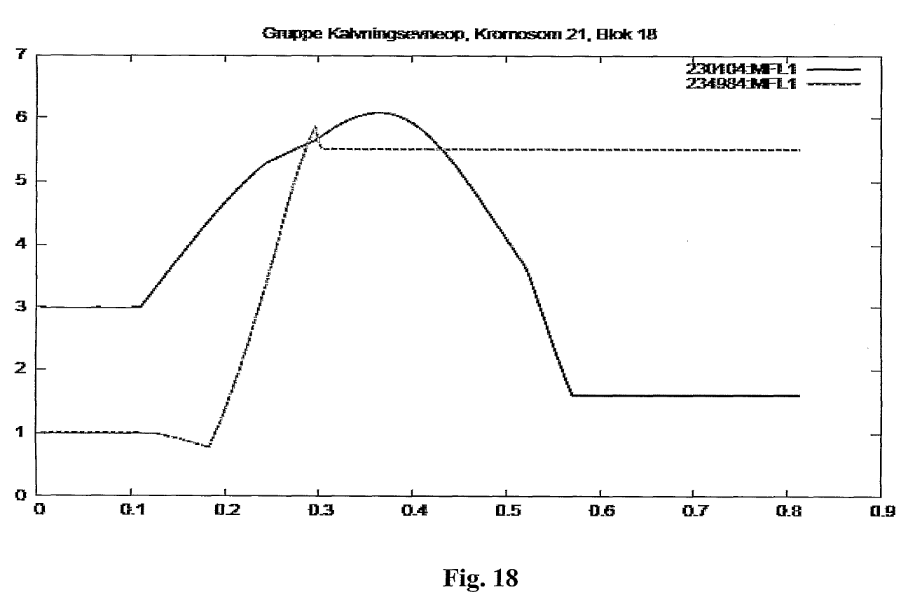

FIG. 19: Genome scan of BTA22 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

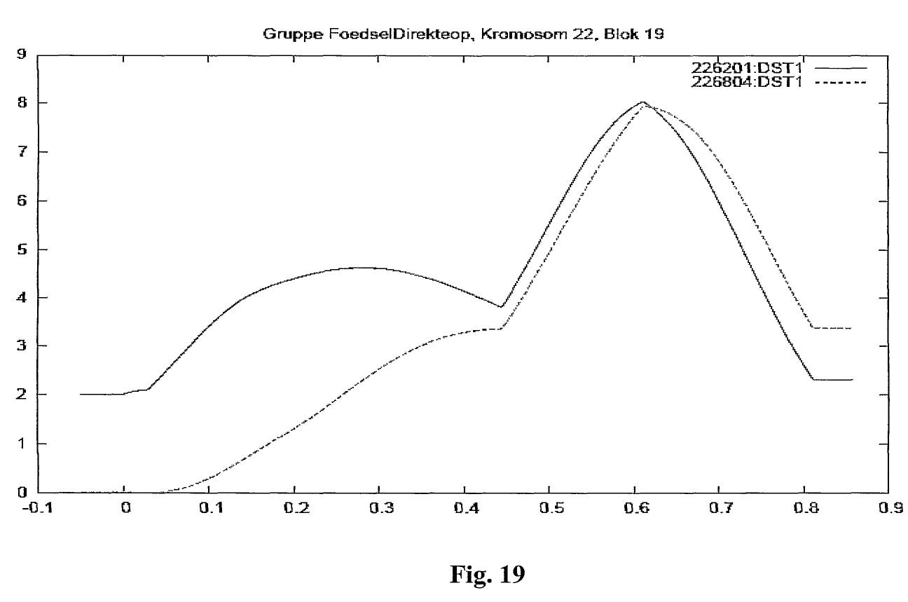

FIG. 20: Genome scan of BTA22 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

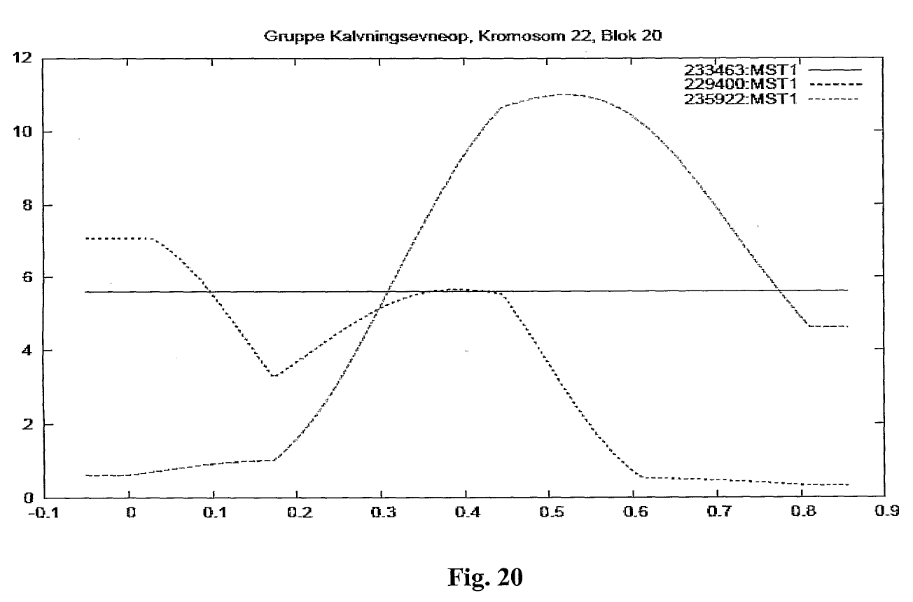

FIG. 21: Genome scan of BTA24 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

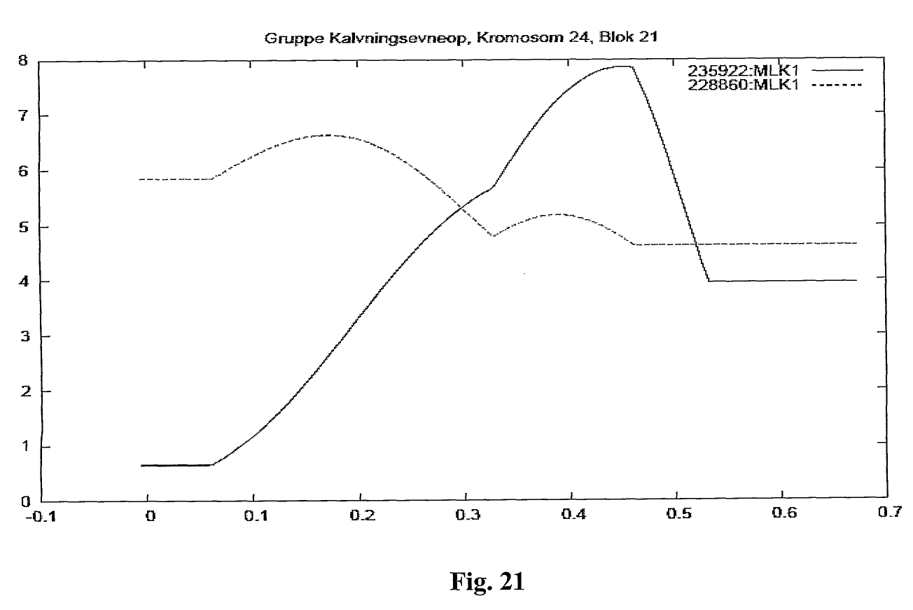

FIG. 22: Genome scan of BTA25 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

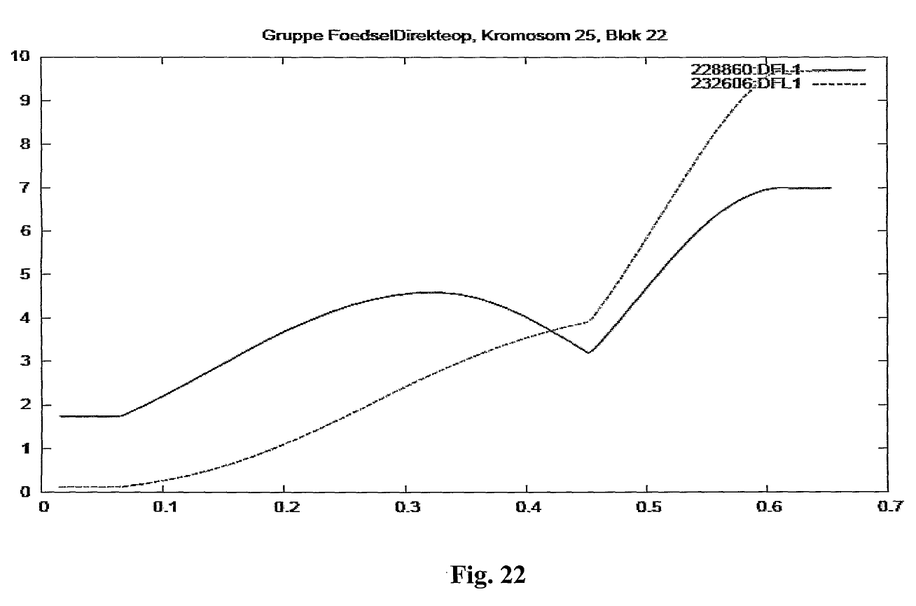

FIG. 23: Genome scan of BTA25 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

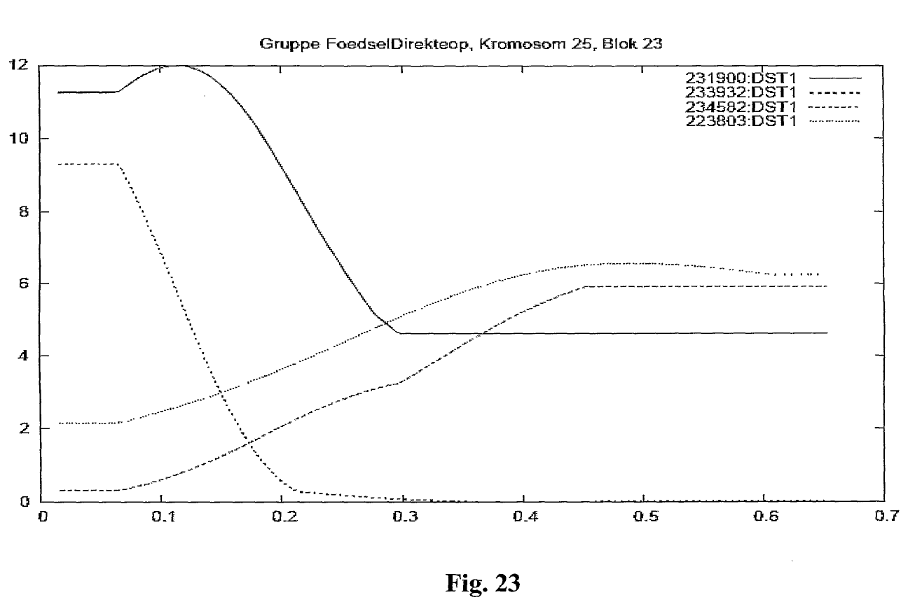

FIG. 24: Genome scan of BTA26 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

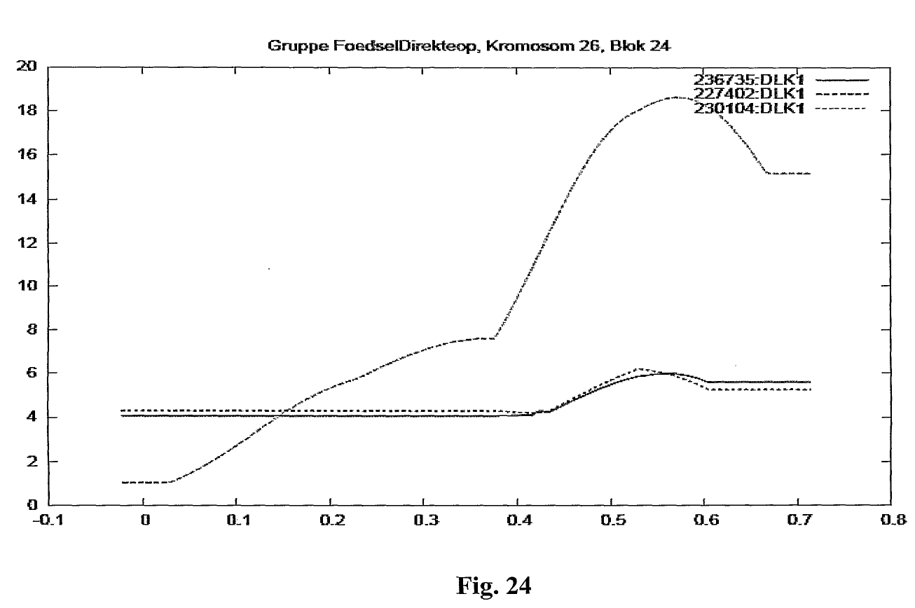

FIG. 25: Genome scan of BTA26 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

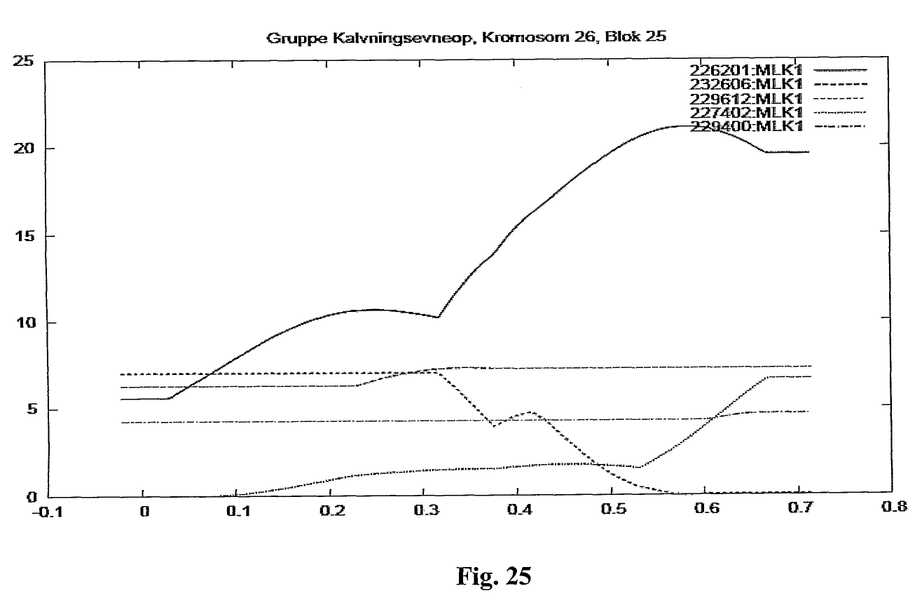

FIG. 26: Genome scan of BTA26 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

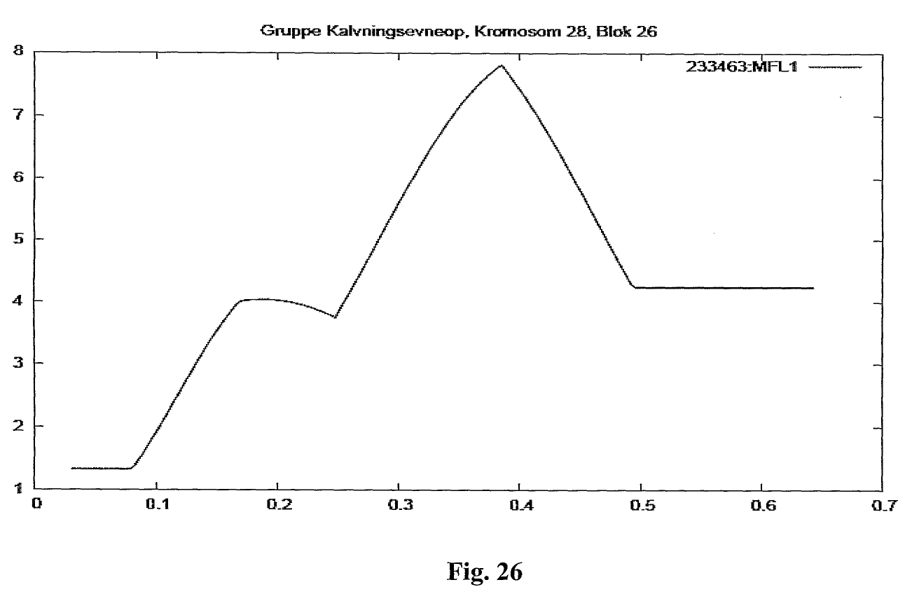

FIG. 27: Genome scan of BTA28 in relation to calving characteristics. Numbers refer to ‘herdbook number’ and calving parameter, respectively. Calving parameters are designated by D: Direct effect, M Maternal effect, while LK corresponds to stillbirth, FL correspond to calving difficulty, and ST correspond to calf size. The number 1 in calving parameter designates that data is derived from first calving. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

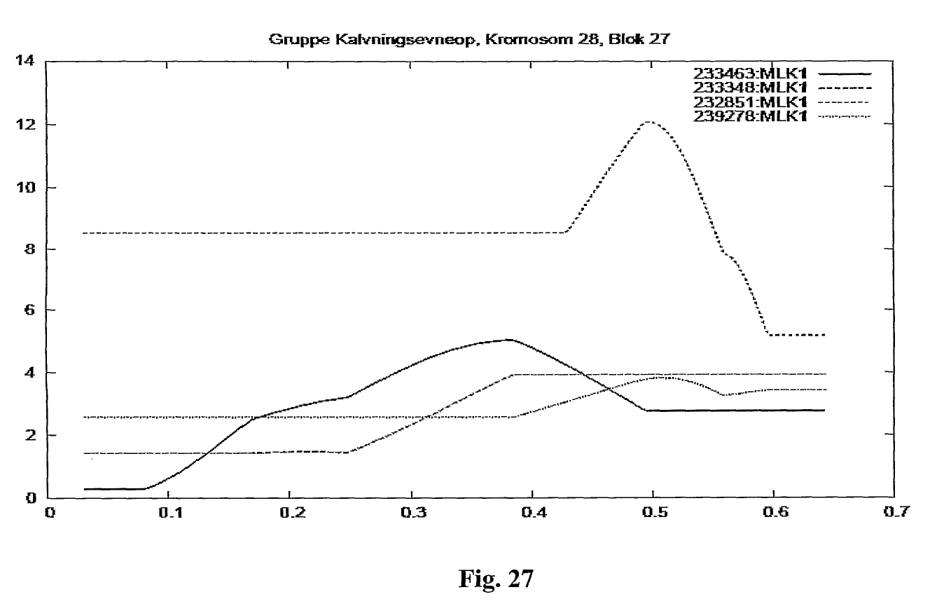

FIG. 28: Genome scan of BTA5 in relation to calving characteristics. Numbers refer to ‘herdbook number’ and calving parameter, respectively. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

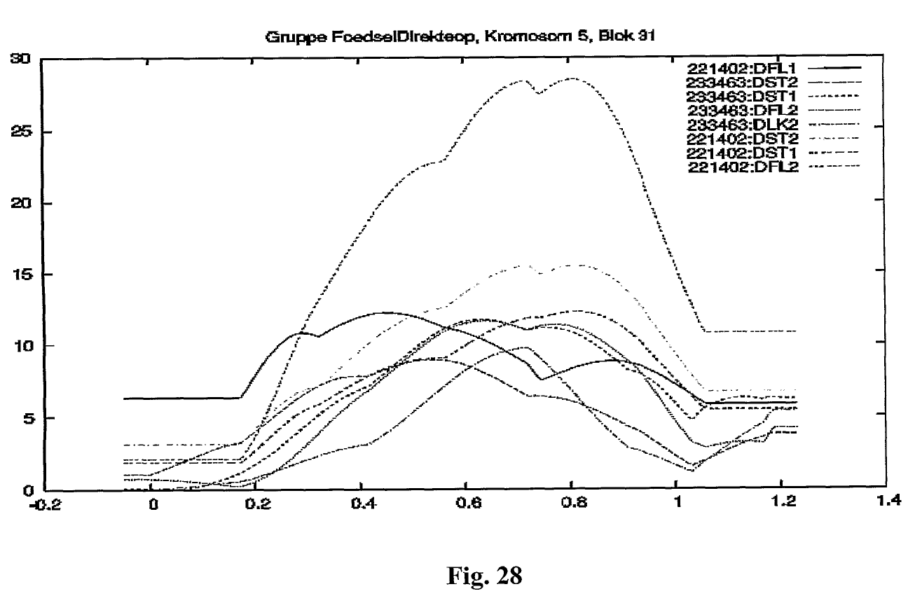

FIG. 29: Genome scan of BTA11 in relation to calving characteristics. Numbers refer to ‘herdbook number’ and calving parameter, respectively. The X-axis represents the distance of the chromosome expressed in Morgan according to the positions employed in this analysis. The Y-axis represents the test-statistics of the QTL analysis expressed in the F-value. High F-values are indicative of genes, which affect the investigated calving traits.

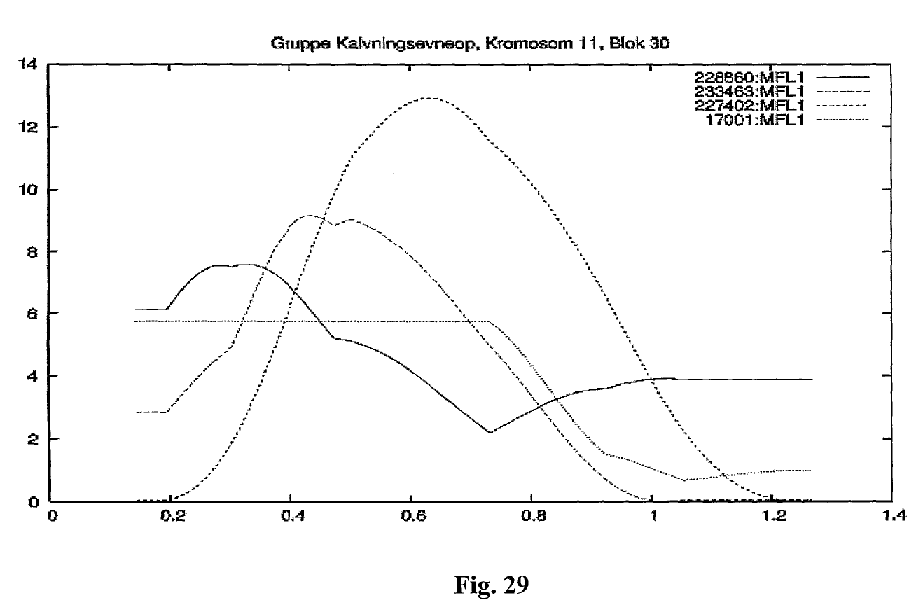

### DETAILED DESCRIPTION OF THE INVENTION

The present invention relates to genetic determinants of calving characteristics in dairy cattle. Calving traits, such as calving difficulties, stillbirths and calf size are economically important factors in the dairy industry. Therefore, it is of economic interest to identity those bovine subjects that have a genetic predisposition for specific calving characteristics. Bovine subjects with genetic predisposition for calving characteristics are carriers of non-desired traits, which both complicate calving, and can be passed on to their offspring.

The term “bovine subject” refers to cattle of any breed and is meant to include both cows and bulls, whether adult or newborn animals. No particular age of the animals are denoted by this term. One example of a bovine subject is a member of the Holstein breed. In one embodiment, the bovine subject is a member of the Holstein-Friesian cattle population. In another embodiment, the bovine subject is a member of the Holstein Swartbont cattle population. In another embodiment, the bovine subject is a member of the Deutsche Holstein Schwarzbunt cattle population. In another embodiment, the bovine subject is a member of the US Holstein cattle population. In one embodiment, the bovine subject is a member of the Red and White Holstein breed. In another embodiment, the bovine subject is a member of the Deutsche Holstein Schwarzbunt cattle population. In one embodiment, the bovine subject is a member of any family, which include members of the Holstein breed. In one embodiment the bovine subject is a member of the Danish Red population. In another embodiment the bovine subject is a member of the Finnish Ayrshire population. In yet another embodiment the bovine subject is a member of the Swedish Red population. In a further embodiment the bovine subject is a member of the Danish Holstein population. In another embodiment, the bovine subject is a member of the Swedish Red and White population. In yet another embodiment, the bovine subject is a member of the Nordic Red population.

In one embodiment of the present invention, the bovine subject is selected from the group consisting of Swedish Red and White, Danish Red, Finnish Ayrshire, Holstein-Friesian, Danish Holstein and Nordic Red. In another embodiment of the present invention, the bovine subject is selected from the group consisting of Finnish Ayrshire and Swedish Red cattle. In another embodiment of the present invention, the bovine subject is selected from the group consisting of Finnish Ayrshire and Swedish Red cattle.

In one embodiment, the bovine subject is selected from the group of breeds shown in table 1a

TABLE 1a
Breed names and breed codes assigned by ICAR
(International Committee for Animal Recording)

| | Breed | National Breed |
|---|---|---|
| Breed | Code | Names Annex |
| Abondance | AB | — |
| Tyrol Grey | AL | 2.2 |
| Angus | AN | 2.1 |
| Aubrac | AU | |
| Ayrshire | AY | 2.1 |
| Belgian Blue | BB | |
| Blonde d'Aquitaine | BD | |
| Beefmaster | BM | |
| Braford | BO | |
| Brahman | BR | |
| Brangus | BN | |
| Brown Swiss | BS | 2.1 |
| Chianina | CA | |
| Charolais | CH | |
| Dexter | DR | |
| Galloway | GA | 2.2 |
| Guernsey | GU | |
| Gelbvieh | GV | |
| Hereford, horned | HH | |
| Hereford, polled | HP | |
| Highland Cattle | HI | |
| Holstein | HO | 2.2 |
| Jersey | JE | |
| Limousin | LM | |
| Maine-Anjou | MA | |
| Murray-Grey | MG | |
| Montbéliard | MO | |
| Marchigiana | MR | |
| Normandy | NO** | |
| Piedmont | PI | 2.2 |
| Pinzgau | PZ | |
| European Red Dairy Breed | [RE]* | 2.1, 2.2 |
| Romagnola | RN | |
| Holstein, Red and White | RW*** | 2.2 |
| Salers | SL** | |
| Santa Gertrudis | SG | |
| South Devon | SD | |
| Shorthorn | [SH]* | 2.2 |
| Simmental | SM | 2.2 |
| Sahiwal | SW | |
| Tarentaise | TA | |
| Welsh Black | WB | |
| Buffalo (Bubalis bubalis) | BF | |

*new breed code
**change from earlier code because of existing code in France
***US proposal WW

In one embodiment, the bovine subject is a member of a breed selected from the group of breeds shown in table 1b

TABLE 1b
Breed names
National Breed Names

| English Name | National names | |
|---|---|---|
| Angus | Including | Aberdeen Angus |
| | | Canadian Angus |
| | | American Angus |
| | | German Angus |
| Ayrshire | Including | Ayrshire in |
| | | Australia |
| | | Canada |
| | | Colombia |
| | | Czech Republic |
| | | Finland |
| | | Kenya |
| | | New Zealand |
| | | Norway (NRF) |
| | | Russia |
| | | South Africa |
| | | Sweden (SRB) and SAB |
| | | UK |
| | | US |
| | | Zimbabwe |
| Belgian Blue | French: | Blanc-bleu Belge |
| | Flemish: | Witblauw Ras van Belgie |
| Brown Swiss | German: | Braunvieh |
| | Italian: | Razza Bruna |
| | French: | Brune |
| | Spanish: | Bruna, Parda Alpina |
| | Serbo-Croatian: | Solvenacko belo |
| | Czech: | Hnedy Karpatsky |
| | Romanian: | Shivitskaja |
| | Russian: | Bruna |
| | Bulgarian: | B'ljarska kafyava |
| European Red Dairy Breed | Including | Danish Red |
| | | Angeln |
| | | Swedish Red and White |
| | | Norwegian Red and White |
| | | Estonian Red |
| | | Latvian Brown |
| | | Lithuranian Red |
| | | Byelorus Red |
| | | Polish Red Lowland |

In one embodiment, the bovine subject is a member of a breed selected from the group of breeds shown in table 1c

TABLE 1c
Breed names
National Breed Names

| English Name | National names | |
|---|---|---|
| European Red Dairy Breed | | Ukrainian Polish Red |
| (continued) | | (French Rouge Flamande?) |
| | | (Belgian Flamande Rouge?) |
| Galloway: | Including | Black and Dun |
| | | Galloway |
| | | Belted Galloway |
| | | Red Galloway |
| | | White Galloway |
| Holstein, Black and White: | Dutch: | Holstein Swartbout |
| | German: | Deutsche Holstein, schwarzbunt |
| | Danish: | Sortbroget Dansk Malkekvaeg |
| | British: | Holstein Friesian |
| | Swedish: | Svensk Låglands Boskaap |
| | French: | Prim Holstein |
| | Italian: | Holstein Frisona |
| | Spanish: | Holstein Frisona |
| Holstein, Red and White | Dutch: | Holstein, roodbunt |
| | German: | Holstein, rotbunt |
| | Danish: | Roedbroget Dansk Malkekvaeg |
| Piedmont | Italian: | Piemontese |
| Shorthorn | Including | Dairy Shorthorn |
| | | Beef Shorthorn |
| | | Polled Shorthorn |
| Simmental | Including dual purpose and beef use | |
| | German: | Fleckvieh |
| | French: | Simmental Française |
| | Italian: | Razza Pezzata Rossa |
| | Czech: | Cesky strakatý |
| | Slovakian: | Slovensky strakaty |
| | Romanian: | Baltata româneasca |
| | Russian: | Simmentalskaja |
| Tyrol Grey | German: | Tiroler Grauvieh |
| | | Oberinntaler Grauvieh |
| | | Rätisches Grauvieh |
| | Italian: | Razza Grigia Alpina |

The term “genetic marker” refers to a variable nucleotide sequence (polymorphism) of the DNA on the bovine chromosome. The variable nucleotide sequence can be identified by methods known to a person skilled in the art, for example by using specific oligonucleotides in for example amplification methods and/or hybridization techniques and/or observation of a size difference. However, the variable nucleotide sequence may also be detected by sequencing or for example restriction fragment length polymorphism analysis. The variable nucleotide sequence may be represented by a deletion, an insertion, repeats, and/or a point mutation. Thus, a genetic marker comprises a variable number of polymorphic alleles.

One type of genetic marker is a microsatellite marker that is linked to a quantitative trait locus. Microsatellite markers refer to short sequences repeated after each other. In short sequences are for example one nucleotide, such as two nucleotides, for example three nucleotides, such as four nucleotides, for example five nucleotides, such as six nucleotides, for example seven nucleotides, such as eight nucleotides, for example nine nucleotides, such as ten nucleotides. However, changes sometimes occur and the number of repeats may increase or decrease. The specific definition and locus of the polymorphic microsatellite markers can be found in the USDA genetic map (Kappes et al. 1997; or by following the link to U.S. Meat Animal Research Center http://www.marc.usda.gov/).

In one embodiment of the present invention, specific marker alleles are linked to quantitative trait loci affecting calving characteristics.

It is furthermore appreciated that the nucleotide sequences of the genetic markers of the present invention are genetically linked to traits for calving in a bovine subject. Consequently, it is also understood that a number of genetic markers may be generated from the nucleotide sequence of the DNA region(s) flanked by and including the genetic markers according to the method of the present invention.

### Calving Trait Characteristics

Calving in a bovine subject is affected by a number of characteristics. Traits that affect calving according to the present invention are for example the occurrence of stillbirth (SB), calving difficulty (CD) and the size of the calf at birth (CS). The traits are assessed by a direct effect (D) of the sire in the calf. However, the traits are also assessed as a maternal effect (M) of the sire in the mother of the calf.

By the term calving characteristics is meant traits which affect calving in the bovine subject or its off-spring. Thus, calving characteristics of a bull are physically manifested by its off-spring—both female and male.

In the present invention calving characteristics comprise the traits SB, CD, and CS, which refer to the following characteristics:

SB: Designates stillbirths.
CS: Size of calves.
CD: Calving difficulties, which are based on registrations from the farmers where it is subjectively registered how difficult the calving is. The calving difficulties consist of four categories:
1: easy with no help
2: easy with assistance
3: difficult but without veterinary assistance
4: difficult with veterinary assistance

In one embodiment of the present invention, the method and kit described herein relates to still births, calving difficulties as categorized herein and/or calf size. In one embodiment of the present invention, the method and kit described herein relates to still births. In another embodiment, the method and kit of the present invention pertains to calving difficulties, such as detected by the calving difficulty categories described above. In yet another embodiment, the method and kit of the present invention relates to calf size. In another embodiment of the present invention, the method and kit described herein relates to any combination of still birth, calving difficulties and/or calf size.

### Granddaughter Design

The granddaughter design includes analysing data from DNA-based markers for grandsires that have been used extensively in breeding and for sons of grandsires where the sons have produced offspring. The phenotypic data that are to be used together with the DNA-marker data are derived from the daughters of the sons. Such phenotypic data could be for example milk production features, features relating to calving, meat quality, or disease. One group of daughters has inherited one allele from their father whereas a second group of daughters has inherited the other allele from their father. By comparing data from the two groups information can be gained whether a fragment of a particular chromosome is harbouring one or more genes that affect the trait in question. It may be concluded whether a QTL is present within this fragment of the chromosome.

A prerequisite for performing a granddaughter design is the availability of detailed phenotypic data. In the present invention such data have been available (http://www.Ir.dk/kvaeq/diverse/principles.pdf).

In contrast, DNA markers can be used directly to provide information of the traits passed on from parents to one or more of their offspring when a number of DNA markers on a chromosome have been determined for one or both parents and their offspring. The markers may be used to calculate the genetic history of the chromosome linked to the DNA markers.

### Frequency of Recombination

The frequency of recombination is the likelihood that a recombination event will occur between two genes or two markers. The frequency of recombination may be calculated as the genetic distance between the two genes or the two markers. Genetic distance is measured in units of centiMorgan (cM). One centiMorgan is equal to a 1% chance that a marker at one genetic locus will be separated from a marker at a second locus due to crossing over in a single generation. One centiMorgan is equivalent, on average, to one million base pairs.

### Chromosomal Regions and Markers

BTA is short for Bos taurus autosome.

One aspect of the present invention relates to a method of determining calving characteristics in a bovine subject, comprising detecting in a sample from said bovine subject the presence or absence of at least one genetic marker that is linked to at least one trait indicative of increased risk of stillbirth and/or increased risk of calving difficulties and/or increased risk of non-desired calf size, wherein said at least one genetic marker is located on the bovine chromosome BTA3 in a region flanked by and including polymorphic microsatellite markers INRA006 and BM7225 and/or BTA4 in the region flanked by and including polymorphic microsatellite markers BMS1788 and MGTG4B and/or, BTA5 in the region flanked by and including polymorphic microsatellite markers BMS1095 and BM2830 and/or, BTA7 in a region flanked by and including polymorphic microsatellite markers BM7160 and BL1043 and/or, BTA8 in a region flanked by and including polymorphic microsatellite markers IDVGA-11 and BMS836 and/or, BTA9 in a region flanked by and including polymorphic microsatellite markers BMS2151 and BMS1967 and/or, BTA10 in a region flanked by and including polymorphic microsatellite markers DIK2658 and BMS2614 and/or, BTA11 in the region flanked by and including polymorphic microsatellite markers BM716 and HEL13 and/or, BTA12 in a region flanked by and including polymorphic microsatellite markers BMS410 and BMS2724 and/or, BTA15 in a region flanked by and including polymorphic microsatellite markers BR3510 and BMS429 and/or, BTA18 in a region flanked by and including polymorphic microsatellite markers IDVGA-31 and DIK4013 and/or, BTA19 in a region flanked by and including polymorphic microsatellite markers BM9202 and BMS601 and/or, BTA20 in a region flanked by and including polymorphic microsatellite markers BM3517 and UWCA26 and/or, BTA21 in a region flanked by and including polymorphic microsatellite markers DIK5182 and IDVGA-30 and/or, BTA22 in a region flanked by and including polymorphic microsatellite markers CSSM26 and BM4102 and/or, BTA24 in a region flanked by and including polymorphic microsatellite markers BMS917 and BMS3024 and/or, BTA25 in a region flanked by and including polymorphic microsatellite markers ILSTS102 and AF5 and/or, BTA26 in a region flanked by and including polymorphic microsatellite markers BMS651 and BM7237 and/or, BTA28 in a region flanked by and including polymorphic microsatellite markers, BMC6020 and BMC2208, wherein the presence of said at least one genetic marker is indicative of calving characteristics of said bovine subject and/or off-spring therefrom.

In order to determine calving characteristics in a bovine subject, wherein the at least one genetic marker is located on a bovine chromosome in the region flanked by and including the polymorphic microsatellite marker, it is appreciated that more than one genetic marker may be employed in the present invention. For example the at least one genetic marker may be a combination of at least two or more genetic markers such that the accuracy may be increased, such as at least three genetic markers, for example four genetic markers, such as at least five genetic markers, for example six genetic markers, such as at least seven genetic markers, for example eight genetic markers, such as at least nine genetic markers, for example ten genetic markers.

The at least one genetic marker may be located on at least one bovine chromosome, such as two chromosomes, for example three chromosomes, such as four chromosomes, for example five chromosomes, and/or such as six chromosomes.

In a preferred embodiment the at least one marker is selected from any of the individual markers of the tables shown herein.

### BTA3

In one embodiment of the invention the at least one genetic marker is located on the bovine chromosome BTA3. In one specific embodiment of the present invention, the at least one genetic marker is located in the region from about 17.1 cM to about 101.8 cM (http://www.marc.usda.gov/) on the bovine chromosome BTA3. In one embodiment the at least one genetic marker is located on the bovine chromosome BTA3 in the region flanked by and including the markers INRA006 and BM7225. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 2a:

TABLE 2a

| | Relative position (cM) |
|---|---|
| Marker on BTA3 | http://www.marc.usda.gov/ |
| INRA006 | 17.1 |
| UWCA7 | 17.4 |
| ILSTS096 | 27.4 |
| DIK4403 | 32.5 |
| RME23 | 32.5 |
| BMS963 | 32.9 |
| BMS819 | 33.5 |
| FCGR1 | 34.6 |
| BL41 | 43.3 |
| DIK4353 | 52.5 |
| INRA003 | 59.4 |
| BMS2790 | 62.4 |
| ILSTS029 | 64.9 |
| BM220 | 66.3 |
| INRA123 | 66.3 |
| BMS862 | 67.4 |
| HUJ246 | 68.0 |
| BMS937 | 68.0 |
| DIK4664 | 68.3 |
| DIK2702 | 77.6 |
| HUJII77 | 87.3 |
| DIK2686 | 95.5 |
| BM7225 | 101.8 |

In a preferred embodiment of the invention, the at least one genetic marker is located in the region from about 34.6 cM to about 87.3 cM (http://www.marc.usda.gov/) on the bovine chromosome BTA3. In one embodiment the at least one genetic marker is located on the bovine chromosome BTA3 in the region flanked by and including the markers FCGR1 and HUJII77. The at least one genetic marker is selected from the group of markers shown in Table 2b:

TABLE 2b

| | Relative position (cM) |
|---|---|
| Marker on BTA3 | http://www.marc.usda.gov/ |
| FCGR1 | 34.6 |
| BL41 | 43.3 |
| DIK4353 | 52.5 |
| INRA003 | 59.4 |
| BMS2790 | 62.4 |
| ILSTS029 | 64.9 |
| BM220 | 66.3 |
| INRA123 | 66.3 |
| BMS862 | 67.4 |
| HUJ246 | 68.0 |
| BMS937 | 68.0 |
| DIK4664 | 68.3 |
| DIK2702 | 77.6 |
| HUJII77 | 87.3 |

In a preferred embodiment of the invention, the at least one genetic marker is located in the region from about 32.5 cM to about 59.4 cM (http://www.marc.usda.gov/) on the bovine chromosome BTA3. In one embodiment the at least one genetic marker is located on the bovine chromosome BTA3 in the region flanked by and including the markers DIK4403 and INRA003. The at least one genetic marker is selected from the group of markers shown in Table 2c:

TABLE 2c

| | Relative position (cM) |
|---|---|
| Marker on BTA3 | http://www.marc.usda.gov/ |
| DIK4403 | 32.5 |
| RME23 | 32.5 |
| BMS963 | 32.9 |
| BMS819 | 33.5 |
| FCGR1 | 34.6 |
| BL41 | 43.3 |
| DIK4353 | 52.5 |
| INRA003 | 59.4 |

In a preferred embodiment of the invention, the at least one genetic marker is located in the region from about 77.6 cM to about 101.8 cM (http://www.marc.usda.gov/) on the bovine chromosome BTA3. In one embodiment the at least one genetic marker is located on the bovine chromosome BTA3 in the region flanked by and including the markers DIK2702 and BM7225. The at least one genetic marker is selected from the group of markers shown in Table 2d:

TABLE 2d

| | Relative position (cM) |
|---|---|
| Marker on BTA3 | http://www.marc.usda.gov/ |
| DIK2702 | 77.6 |
| HUJII77 | 87.3 |
| DIK2686 | 95.5 |
| BM7225 | 101.8 |

In a preferred embodiment of the invention, the at least one genetic marker is located in the region from about 52.5 cM to about 68.3 cM (http://www.marc.usda.gov/) on the bovine chromosome BTA3. In one embodiment the at least one genetic marker is located on the bovine chromosome BTA3 in the region flanked by and including the markers DIK4353 and DIK4664. The at least one genetic marker is selected from the group of markers shown in Table 2e:

TABLE 2e

| | Relative position (cM) |
|---|---|
| Marker on BTA3 | http://www.marc.usda.gov/ |
| DIK4353 | 52.5 |
| INRA003 | 59.4 |
| BMS2790 | 62.4 |
| ILSTS029 | 64.9 |
| BM220 | 66.3 |
| INRA123 | 66.3 |
| BMS862 | 67.4 |
| HUJ246 | 68.0 |
| BMS937 | 68.0 |
| DIK4664 | 68.3 |

In a preferred embodiment of the invention, the at least one genetic marker is located in the region from about 59.4 cM to about 66.3 cM (http://www.marc.usda.gov/) on the bovine chromosome BTA3. In one embodiment the at least one genetic marker is located on the bovine chromosome BTA3 in the region flanked by and including the markers INRA003 and INRA123. The at least one genetic marker is selected from the group of markers shown in Table 2f:

TABLE 2f

| | Relative position (cM) |
|---|---|
| Marker on BTA3 | http://www.marc.usda.gov/ |
| INRA003 | 59.4 |
| BMS2790 | 62.4 |
| ILSTS029 | 64.9 |
| BM220 | 66.3 |
| INRA123 | 66.3 |

In a preferred embodiment of the invention, the at least one genetic marker is located in the region from about 32.5 cM to about 52.5 cM (http://www.marc.usda.gov/) on the bovine chromosome BTA3. In one embodiment the at least one genetic marker is located on the bovine chromosome BTA3 in the region flanked by and including the markers DIK4403 and DIK4353. The at least one genetic marker is selected from the group of markers shown in Table 2g:

TABLE 2g

| | Relative position (cM) |
|---|---|
| Marker on BTA3 | http://www.marc.usda.gov/ |
| DIK4403 | 32.5 |
| RME23 | 32.5 |
| BMS963 | 32.9 |
| BMS819 | 33.5 |
| FCGR1 | 34.6 |
| BL41 | 43.3 |
| DIK4353 | 52.5 |

In a preferred embodiment of the invention, the at least one genetic marker is located in the region from about 77.6 cM to 101.8 cM (http://www.marc.usda.gov/) on the bovine chromosome BTA3. In one embodiment the at least one genetic marker is located on the bovine chromosome BTA3 in the region flanked by and including the marker FCGR1 and HUJII77. The at least one genetic marker is selected from the group of markers shown in Table 2h:

TABLE 2h

| | Relative position (cM) |
|---|---|
| Marker on BTA3 | http://www.marc.usda.gov/ |
| DIK2702 | 77.6 |
| HUJII77 | 87.3 |
| DIK2686 | 95.5 |
| BM7225 | 101.8 |

### BTA4

In one embodiment of the invention the at least one genetic marker is located on the bovine chromosome BTA4. In one specific embodiment of the present invention, the at least one genetic marker is located in the region from about 12.5 cM to about 112.8 cM (http://www.marc.usda.gov/) on the bovine chromosome BTA4. In one embodiment the at least one genetic marker is located on the bovine chromosome BTA4 in the region flanked by and including the markers BMS1788 and MGTG4B. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 3a:

TABLE 3a

| | Relative position (cM) |
|---|---|
| Marker on BTA4 | http://www.marc.usda.gov/ |
| BMS1788 | 12.5 |
| BMS2646 | 43.2 |
| TGLA116 | 52.5 |
| INRA072 | 63.0 |
| BM8233 | 73.4 |
| BMS648 | 91.2 |
| BR6303 | 104.9 |
| MGTG4B | 112.8 |

In a preferred embodiment of the invention, the at least one genetic marker is located in the region from about 12.5 cM to about 91.2 cM (http://www.marc.usda.gov/) on the bovine chromosome BTA4. In one embodiment the at least one genetic marker is located on the bovine chromosome BTA4 in the region flanked by and including the markers BMS1788 and BMS648. The at least one genetic marker is selected from the group of markers shown in Table 3b:

TABLE 3b

| | Relative position (cM) |
|---|---|
| Marker on BTA4 | http://www.marc.usda.gov/ |
| BMS1788 | 12.5 |
| BMS2646 | 43.2 |
| TGLA116 | 52.5 |
| INRA072 | 63.0 |
| BM8233 | 73.4 |
| BMS648 | 91.2 |

In a preferred embodiment of the invention, the at least one genetic marker is located in the region from about 43.2 cM to about 91.2 cM (http://www.marc.usda.gov/) on the bovine chromosome BTA4. In one embodiment the at least one genetic marker is located on the bovine chromosome BTA4 in the region flanked by and including the markers BMS2646 and BMS648. The at least one genetic marker is selected from the group of markers shown in Table 3c:

TABLE 3c

| | Relative position (cM) |
|---|---|
| Marker on BTA4 | http://www.marc.usda.gov/ |
| BMS2646 | 43.2 |
| TGLA116 | 52.5 |
| INRA072 | 63.0 |
| BM8233 | 73.4 |
| BMS648 | 91.2 |

In a preferred embodiment of the invention, the at least one genetic marker is located in the region from about 43.2 cM to about 63.0 cM (http://www.marc.usda.gov/) on the bovine chromosome BTA4. In one embodiment the at least one genetic marker is located on the bovine chromosome BTA4 in the region flanked by and including the markers BMS2646 and INRA072. The at least one genetic marker is selected from the group of markers shown in Table 3d:

TABLE 3d

| | Relative position (cM) |
|---|---|
| Marker on BTA4 | http://www.marc.usda.gov/ |
| BMS2646 | 43.2 |
| TGLA116 | 52.5 |
| INRA072 | 63.0 |

In a preferred embodiment of the invention, the at least one genetic marker is located in the region from about 52.2 cM to about 73.4 cM (http://www.marc.usda.gov/) on the bovine chromosome BTA4. In one embodiment the at least one genetic marker is located on the bovine chromosome BTA4 in the region flanked by and including the markers TGLA116 and BM8233. The at least one genetic marker is selected from the group of markers shown in Table 3e:

TABLE 3e

| | Relative position (cM) |
|---|---|
| Marker on BTA4 | http://www.marc.usda.gov/ |
| TGLA116 | 52.5 |
| INRA072 | 63.0 |
| BM8233 | 73.4 |

In a preferred embodiment of the invention, the at least one genetic marker is located in the region from about 63.0 cM to about 91.2 cM (http://www.marc.usda.gov/) on the bovine chromosome BTA4. In one embodiment the at least one genetic marker is located on the bovine chromosome BTA4 in the region flanked by and including the markers INRA072 and BMS648. The at least one genetic marker is selected from the group of markers shown in Table 3f:

TABLE 3f

| | Relative position (cM) |
|---|---|
| Marker on BTA4 | http://www.marc.usda.gov/ |
| INRA072 | 63.0 |
| BM8233 | 73.4 |
| BMS648 | 91.2 |

In a preferred embodiment of the invention, the at least one genetic marker is located in the region from about 63.0 cM to about 73.4 cM (http://www.marc.usda.gov/) on the bovine chromosome BTA4. In one embodiment the at least one genetic marker is located on the bovine chromosome BTA4 in the region flanked by and including the markers INRA072 and BM8233. The at least one genetic marker is selected from the group of markers shown in Table 3g:

TABLE 3g

| | Relative position (cM) |
|---|---|
| Marker on BTA4 | http://www.marc.usda.gov/ |
| INRA072 | 63.0 |
| BM8233 | 73.4 |

### BTA5

In one embodiment of the invention the at least one genetic marker is located on the bovine chromosome BTA5. In one specific embodiment of the present invention, the at least one genetic marker is located in the region from about 0.0 cM to about 116.9 cM (http://www.marc.usda.gov/) on the bovine chromosome BTA5. In one embodiment the at least one genetic marker is located on the bovine chromosome BTA5 in the region flanked by and including the markers BMS1095 and BM2830. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 4a:

TABLE 4a

| | Relative position (cM) |
|---|---|
| Marker on BTA5 | http://www.marc.usda.gov/ |
| BMS1095 | 0.0 |
| BM6026 | 6.0 |
| MNB-33 | 7.4 |
| BMS610 | 12.0 |
| BP1 | 17.3 |
| DIK4747 | 18.3 |
| DIK2718 | 30.1 |
| AGLA293 | 32.3 |
| DIK5002 | 33.7 |
| DIK4759 | 40.3 |
| BMC1009 | 41.7 |
| CSSM034 | 45.5 |
| RM500 | 56.3 |
| BMS1617 | 56.3 |
| DIK5046 | 66.2 |
| ETH10 | 71.8 |
| CSSM022 | 74.2 |
| BMS1216 | 78.2 |
| DIK2943 | 82.9 |
| BMS1248 | 90.8 |
| BM315 | 103.2 |
| BMS1658 | 105.7 |
| BM2830 | 116.9 |

In a preferred embodiment of the invention, the at least one genetic marker is located in the region from about 0.0 cM to about 103.2 cM (http://www.marc.usda.gov/) on the bovine chromosome BTA5. In one embodiment the at least one genetic marker is located on the bovine chromosome BTA5 in the region flanked by and including the markers BMS1095 and BM315. The at least one genetic marker is selected from the group of markers shown in Table 4b:

TABLE 4b

| | Relative position (cM) |
|---|---|
| Marker on BTA5 | http://www.marc.usda.gov/ |
| BMS1095 | 0.0 |
| BM6026 | 6.0 |
| MNB-33 | 7.4 |
| BMS610 | 12.0 |
| BP1 | 17.3 |
| DIK4747 | 18.3 |
| DIK2718 | 30.1 |
| AGLA293 | 32.3 |
| DIK5002 | 33.7 |
| DIK4759 | 40.3 |
| BMC1009 | 41.7 |
| CSSM034 | 45.5 |
| RM500 | 56.3 |
| BMS1617 | 56.3 |
| DIK5046 | 66.2 |
| ETH10 | 71.8 |
| CSSM022 | 74.2 |
| BMS1216 | 78.2 |
| DIK2943 | 82.9 |
| BMS1248 | 90.8 |
| BM315 | 103.2 |

In a preferred embodiment of the invention, the at least one genetic marker is located in the region from about 30.1 cM to about 103.2 cM (http://www.marc.usda.gov/) on the bovine chromosome BTA5. In one embodiment the at least one genetic marker is located on the bovine chromosome BTA5 in the region flanked by and including the markers DIK2718 and BM315. The at least one genetic marker is selected from the group of markers shown in Table 4c:

TABLE 4c

| | Relative position (cM) |
|---|---|
| Marker on BTA5 | http://www.marc.usda.gov/ |
| DIK2718 | 30.1 |
| AGLA293 | 32.3 |
| DIK5002 | 33.7 |
| DIK4759 | 40.3 |
| BMC1009 | 41.7 |
| CSSM034 | 45.5 |
| RM500 | 56.3 |
| BMS1617 | 56.3 |
| DIK5046 | 66.2 |
| ETH10 | 71.8 |
| CSSM022 | 74.2 |
| BMS1216 | 78.2 |
| DIK2943 | 82.9 |
| BMS1248 | 90.8 |
| BM315 | 103.2 |

In a preferred embodiment of the invention, the at least one genetic marker is located in the region from about 30.1 cM to about 78.2 cM (http://www.marc.usda.gov/) on the bovine chromosome BTA5. In one embodiment the at least one genetic marker is located on the bovine chromosome BTA5 in the region flanked by and including the markers DIK2718 and BMS1216. The at least one genetic marker is selected from the group of markers shown in Table 4d:

TABLE 4d

| | Relative position (cM) |
|---|---|
| Marker on BTA5 | http://www.marc.usda.gov/ |
| DIK2718 | 30.1 |
| AGLA293 | 32.3 |
| DIK5002 | 33.7 |
| DIK4759 | 40.3 |
| BMC1009 | 41.7 |
| CSSM034 | 45.5 |
| RM500 | 56.3 |
| BMS1617 | 56.3 |
| DIK5046 | 66.2 |
| ETH10 | 71.8 |
| CSSM022 | 74.2 |
| BMS1216 | 78.2 |

In a preferred embodiment of the invention, the at least one genetic marker is located in the region from about 18.3 cM to about 56.3 cM (http://www.marc.usda.gov/) on the bovine chromosome BTA5. In one embodiment the at least one genetic marker is located on the bovine chromosome BTA5 in the region flanked by and including the markers DIK4747 and RM500. The at least one genetic marker is selected from the group of markers shown in Table 4e:

TABLE 4e

| | Relative position (cM) |
|---|---|
| Marker on BTA5 | http://www.marc.usda.gov/ |
| DIK4747 | 18.3 |
| DIK2718 | 30.1 |
| AGLA293 | 32.3 |
| DIK5002 | 33.7 |
| DIK4759 | 40.3 |
| BMC1009 | 41.7 |
| CSSM034 | 45.5 |
| RM500 | 56.3 |

In a preferred embodiment of the invention, the at least one genetic marker is located in the region from about 17.3 cM to about 33.7 cM (http://www.marc.usda.gov/) on the bovine chromosome BTA5. In one embodiment the at least one genetic marker is located on the bovine chromosome BTA5 in the region flanked by and including the markers BP1 and DIK5002. The at least one genetic marker is selected from the group of markers shown in Table 4f:

TABLE 4f

| | Relative position (cM) |
|---|---|
| Marker on BTA5 | http://www.marc.usda.gov/ |
| BP1 | 17.3 |
| DIK4747 | 18.3 |
| DIK2718 | 30.1 |
| AGLA293 | 32.3 |
| DIK5002 | 33.7 |

In a preferred embodiment of the invention, the at least one genetic marker is located in the region from about 45.5 cM to about 82.9 cM (http://www.marc.usda.gov/) on the bovine chromosome BTA5. In one embodiment the at least one genetic marker is located on the bovine chromosome BTA5 in the region flanked by and including the markers CSSM034 and DIK2943. The at least one genetic marker is selected from the group of markers shown in Table 4g:

TABLE 4g

| | Relative position (cM) |
|---|---|
| Marker on BTA5 | http://www.marc.usda.gov/ |
| CSSM034 | 45.5 |
| RM500 | 56.3 |
| BMS1617 | 56.3 |
| DIK5046 | 66.2 |
| ETH10 | 71.8 |
| CSSM022 | 74.2 |
| BMS1216 | 78.2 |
| DIK2943 | 82.9 |

In a preferred embodiment of the invention, the at least one genetic marker is located in the region from about 45.5 cM to about 66.2 cM (http://www.marc.usda.gov/) on the bovine chromosome BTA5. In one embodiment the at least one genetic marker is located on the bovine chromosome BTA5 in the region flanked by and including the markers CSSM034 and DIK5046. The at least one genetic marker is selected from the group of markers shown in Table 4h:

TABLE 4h

| | Relative position (cM) |
|---|---|
| Marker on BTA5 | http://www.marc.usda.gov/ |
| CSSM034 | 45.5 |
| RM500 | 56.3 |
| BMS1617 | 56.3 |
| DIK5046 | 66.2 |

In a preferred embodiment of the invention, the at least one genetic marker is located in the region from about 66.2 cM to about 82.9 cM (http://www.marc.usda.gov/) on the bovine chromosome BTA5. In one embodiment the at least one genetic marker is located on the bovine chromosome BTA5 in the region flanked by and including the markers DIK5046 and DIK2943. The at least one genetic marker is selected from the group of markers shown in Table 4i:

TABLE 4i

| | Relative position (cM) |
|---|---|
| Marker on BTA5 | http://www.marc.usda.gov/ |
| DIK5046 | 66.2 |
| ETH10 | 71.8 |
| CSSM022 | 74.2 |
| BMS1216 | 78.2 |
| DIK2943 | 82.9 |

In a preferred embodiment of the invention, the at least one genetic marker is located in the region from about 71.8 cM to about 90.8 cM (http://www.marc.usda.gov/) on the bovine chromosome BTA5. In one embodiment the at least one genetic marker is located on the bovine chromosome BTA5 in the region flanked by and including the markers ETH10 and BMS1248. The at least one genetic marker is selected from the group of markers shown in Table 4j:

TABLE 4j

| | Relative position (cM) |
|---|---|
| Marker on BTA5 | http://www.marc.usda.gov/ |
| ETH10 | 71.8 |
| CSSM022 | 74.2 |
| BMS1216 | 78.2 |
| DIK2943 | 82.9 |
| BMS1248 | 90.8 |

### BTA7

In one embodiment of the invention the at least one genetic marker is located on the bovine chromosome BTA7. In one specific embodiment of the present invention, the at least one genetic marker is located in the region from about 0.0 cM to about 135.6 cM (http://www.marc.usda.gov/) on the bovine chromosome BTA7. In one embodiment the at least one genetic marker is located on the bovine chromosome BTA7 in the region flanked by and including the markers BM7160 and BL1043. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 5a:

TABLE 5a

| | Relative position (cM) |
|---|---|
| Marker on BTA7 | http://www.marc.usda.gov/ |
| BM7160 | 0.0 |
| BL1067 | 14.7 |
| BMS713 | 16.8 |
| DIK5321 | 22.3 |
| DIK4421 | 22.7 |
| DIK2207 | 26.7 |
| DIK5412 | 30.2 |
| IL4 | 32.0 |
| BM6105 | 37.9 |
| TGLA303 | 39.3 |
| DIK2819 | 47.9 |
| DIK4606 | 55.3 |
| BM7247 | 57.3 |
| UWCA20 | 58.6 |
| BM6117 | 62.2 |
| BMS2840 | 65.3 |
| DIK2915 | 76.2 |
| BMS2258 | 77.2 |
| OARAE129 | 95.9 |
| DIK2895 | 103.1 |
| ILSTS006 | 116.6 |
| BL1043 | 135.6 |

In a preferred embodiment of the invention, the at least one genetic marker is located in the region from about 30.2 cM to about 95.9 cM (http://www.marc.usda.gov/) on the bovine chromosome BTA7. In one embodiment the at least one genetic marker is located on the bovine chromosome BTA7 in the region flanked by and including the markers DIK5412 and OARAE129. The at least one genetic marker is selected from the group of markers shown in Table 5b:

TABLE 5b

| | Relative position (cM) |
|---|---|
| Marker on BTA7 | http://www.marc.usda.gov/ |
| DIK5412 | 30.2 |
| IL4 | 32.0 |
| BM6105 | 37.9 |
| TGLA303 | 39.3 |
| DIK2819 | 47.9 |
| DIK4606 | 55.3 |
| BM7247 | 57.3 |
| UWCA20 | 58.6 |
| BM6117 | 62.2 |
| BMS2840 | 65.3 |
| DIK2915 | 76.2 |
| BMS2258 | 77.2 |
| OARAE129 | 95.9 |

In a preferred embodiment of the invention, the at least one genetic marker is located in the region from about 30.2 cM to about 55.3 cM (http://www.marc.usda.gov/) on the bovine chromosome BTA7. In one embodiment the at least one genetic marker is located on the bovine chromosome BTA7 in the region flanked by and including the markers DIK5412 and DIK4606. The at least one genetic marker is selected from the group of markers shown in Table 5c:

TABLE 5c

| | Relative position (cM) |
|---|---|
| Marker on BTA7 | http://www.marc.usda.gov/ |
| DIK5412 | 30.2 |
| IL4 | 32.0 |
| BM6105 | 37.9 |
| TGLA303 | 39.3 |
| DIK2819 | 47.9 |
| DIK4606 | 55.3 |

In a preferred embodiment of the invention, the at least one genetic marker is located in the region from about 58.6 cM to about 95.9 cM (http://www.marc.usda.gov/) on the bovine chromosome BTA7. In one embodiment the at least one genetic marker is located on the bovine chromosome BTA7 in the region flanked by and including the markers UWCA20 and OARAE129. The at least one genetic marker is selected from the group of markers shown in Table 5d:

TABLE 5d

| | Relative position (cM) |
|---|---|
| Marker on BTA7 | http://www.marc.usda.gov/ |
| UWCA20 | 58.6 |
| BM6117 | 62.2 |
| BMS2840 | 65.3 |
| DIK2915 | 76.2 |
| BMS2258 | 77.2 |
| OARAE129 | 95.9 |

In a preferred embodiment of the invention, the at least one genetic marker is located in the region from about 77.2 cM to about 135.6 cM (http://www.marc.usda.gov/) on the bovine chromosome BTA7. In one embodiment the at least one genetic marker is located on the bovine chromosome BTA7 in the region flanked by and including the markers BMS2258 and BL1043. The at least one genetic marker is selected from the group of markers shown in Table 5e:

TABLE 5e

| | Relative position (cM) |
|---|---|
| Marker on BTA7 | http://www.marc.usda.gov/ |
| BMS2258 | 77.2 |
| OARAE129 | 95.9 |
| DIK2895 | 103.1 |
| ILSTS006 | 116.6 |
| BL1043 | 135.6 |

In a preferred embodiment of the invention, the at least one genetic marker is located in the region from about 77.2 cM to about 116.6 cM (http://www.marc.usda.gov/) on the bovine chromosome BTA7. In one embodiment the at least one genetic marker is located on the bovine chromosome BTA7 in the region flanked by and including the markers BMS2258 and ILSTS006. The at least one genetic marker is selected from the group of markers shown in Table 5f:

TABLE 5f

| | Relative position (cM) |
|---|---|
| Marker on BTA7 | http://www.marc.usda.gov/ |
| BMS2258 | 77.2 |
| OARAE129 | 95.9 |
| DIK2895 | 103.1 |
| ILSTS006 | 116.6 |

In a preferred embodiment of the invention, the at least one genetic marker is located in the region from about 77.2 cM to about 95.5 cM (http://www.marc.usda.gov/) on the bovine chromosome BTA7. In one embodiment the at least one genetic marker is located on the bovine chromosome BTA7 in the region flanked by and including the markers BMS2258 and OARAE129. The at least one genetic marker is selected from the group of markers shown in Table 5g:

TABLE 5g

| | Relative position (cM) |
|---|---|
| Marker on BTA7 | http://www.marc.usda.gov/ |
| BMS2258 | 77.2 |
| OARAE129 | 95.9 |

### BTA8

In one embodiment of the invention the at least one genetic marker is located on the bovine chromosome BTA8. In one specific embodiment of the present invention, the at least one genetic marker is located in the region from about 11.3 cM to about 122.9 cM (http://www.marc.usda.gov/) on the bovine chromosome BTA8. In one embodiment the at least one genetic marker is located on the bovine chromosome BTA8 in the region flanked by and including the markers IDVGA-11 and BMS836. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 6a:

TABLE 6a

| | Relative position (cM) |
|---|---|
| Marker on BTA8 | http://www.marc.usda.gov/ |
| IDVGA-11 | 11.3 |
| BMS1591 | 31.4 |
| BMS678 | 41.6 |
| INRA129 | 54.6 |
| BMS2072 | 66.0 |
| BMS887 | 68.5 |
| URB037 | 69.0 |
| MCM64 | 71.1 |
| CSSM047 | 118.7 |
| BMS836 | 122.9 |

In a preferred embodiment of the invention, the at least one genetic marker is located in the region from about 11.3 cM to about 71.1 cM (http://www.marc.usda.gov/) on the bovine chromosome BTA8. In one embodiment the at least one genetic marker is located on the bovine chromosome BTA8 in the region flanked by and including the markers IDVGA-11 and MCM64. The at least one genetic marker is selected from the group of markers shown in Table 6b:

TABLE 6b

| | Relative position (cM) |
|---|---|
| Marker on BTA8 | http://www.marc.usda.gov/ |
| IDVGA-11 | 11.3 |
| BMS1591 | 31.4 |
| BMS678 | 41.6 |
| INRA129 | 54.6 |
| BMS2072 | 66.0 |
| BMS887 | 68.5 |
| URB037 | 69.0 |
| MCM64 | 71.1 |

In a preferred embodiment of the invention, the at least one genetic marker is located in the region from about 41.6 cM to about 66.0 cM (http://www.marc.usda.gov/) on the bovine chromosome BTA8. In one embodiment the at least one genetic marker is located on the bovine chromosome BTA8 in the region flanked by and including the markers BMS678 and BMS2072. The at least one genetic marker is selected from the group of markers shown in Table 6c:

TABLE 6c

| | Relative position (cM) |
|---|---|
| Marker on BTA8 | http://www.marc.usda.gov/ |
| BMS678 | 41.6 |
| INRA129 | 54.6 |
| BMS2072 | 66.0 |

In a preferred embodiment of the invention, the at least one genetic marker is located in the region from about 71.1 cM to about 122.9 cM (http://www.marc.usda.gov/) on the bovine chromosome BTA8. In one embodiment the at least one genetic marker is located on the bovine chromosome BTA8 in the region flanked by and including the markers MCM64 and BMS836. The at least one genetic marker is selected from the group of markers shown in Table 6d:

TABLE 6d

| | Relative position (cM) |
|---|---|
| Marker on BTA8 | http://www.marc.usda.gov/ |
| MCM64 | 71.1 |
| CSSM047 | 118.7 |
| BMS836 | 122.9 |

In a preferred embodiment of the invention, the at least one genetic marker is located in the region from about 11.3 cM to about 41.6 cM (http://www.marc.usda.gov/) on the bovine chromosome BTA8. In one embodiment the at least one genetic marker is located on the bovine chromosome BTA8 in the region flanked by and including the markers IDVGA-11 and BMS678. The at least one genetic marker is selected from the group of markers shown in Table 6e:

TABLE 6e

| | Relative position (cM) |
|---|---|
| Marker on BTA8 | http://www.marc.usda.gov/ |
| IDVGA-11 | 11.3 |
| BMS1591 | 31.4 |
| BMS678 | 41.6 |

### BTA9

In one embodiment of the invention the at least one genetic marker is located on the bovine chromosome BTA9. In one specific embodiment of the present invention, the at least one genetic marker is located in the region from about 8.49 cM to about 109.3 cM (http://www.marc.usda.gov/) on the bovine chromosome BTA9. In one embodiment the at least one genetic marker is located on the bovine chromosome BTA9 in the region flanked by and including the markers BMS2151 and BMS1967. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 7a:

TABLE 7a

| | Relative position (cM) |
|---|---|
| Marker on BTA9 | http://www.marc.usda.gov/ |
| BMS2151 | 8.49 |
| ETH225 | 12.8 |
| ILSTS037 | 26.3 |
| BM2504 | 30.9 |
| DIK2892 | 30.9 |
| DIK3003 | 36.5 |
| DIK3002 | 36.5 |
| BMS1267 | 38.7 |
| DIK5142 | 43.8 |
| BMS555 | 43.8 |
| DIK5364 | 45.7 |
| UWCA9 | 50.0 |
| DIK4720 | 54.0 |
| BMS1290 | 64.9 |
| DIK2816 | 68.1 |
| BM6436 | 77.6 |
| BMS2753 | 79.2 |
| BM4208 | 90.7 |
| BMS2819 | 91.0 |
| BMS2295 | 98.6 |
| BMS1967 | 109.3 |

In a preferred embodiment of the invention, the at least one genetic marker is located in the region from about 12.8 cM to about 90.7 cM (http://www.marc.usda.gov/) on the bovine chromosome BTA9. In one embodiment the at least one genetic marker is located on the bovine chromosome BTA9 in the region flanked by and including the markers ETH225 and BM4208. The at least one genetic marker is selected from the group of markers shown in Table 7b:

TABLE 7b

| | Relative position (cM) |
|---|---|
| Marker on BTA9 | http://www.marc.usda.gov/ |
| ETH225 | 12.8 |
| ILSTS037 | 26.3 |
| BM2504 | 30.9 |
| DIK2892 | 30.9 |
| DIK3003 | 36.5 |
| DIK3002 | 36.5 |
| BMS1267 | 38.7 |
| DIK5142 | 43.8 |
| BMS555 | 43.8 |
| DIK5364 | 45.7 |
| UWCA9 | 50.0 |
| DIK4720 | 54.0 |
| BMS1290 | 64.9 |
| DIK2816 | 68.1 |
| BM6436 | 77.6 |
| BMS2753 | 79.2 |
| BM4208 | 90.7 |

In a preferred embodiment of the invention, the at least one genetic marker is located in the region from about 12.8 cM to about 64.9 cM (http://www.marc.usda.gov/) on the bovine chromosome BTA9. In one embodiment the at least one genetic marker is located on the bovine chromosome BTA9 in the region flanked by and including the markers ETH225 and BMS1290. The at least one genetic marker is selected from the group of markers shown in Table 7c:

TABLE 7c

| | Relative position (cM) |
|---|---|
| Marker on BTA9 | http://www.marc.usda.gov/ |
| ETH225 | 12.8 |
| ILSTS037 | 26.3 |
| BM2504 | 30.9 |
| DIK2892 | 30.9 |
| DIK3003 | 36.5 |
| DIK3002 | 36.5 |
| BMS1267 | 38.7 |
| DIK5142 | 43.8 |
| BMS555 | 43.8 |
| DIK5364 | 45.7 |
| UWCA9 | 50.0 |
| DIK4720 | 54.0 |
| BMS1290 | 64.9 |

In a preferred embodiment of the invention, the at least one genetic marker is located in the region from about 50.0 cM to about 91.0 cM (http://www.marc.usda.gov/) on the bovine chromosome BTA9. In one embodiment the at least one genetic marker is located on the bovine chromosome BTA9 in the region flanked by and including the markers UWCA9 and BMS2819. The at least one genetic marker is selected from the group of markers shown in Table 7d:

TABLE 7d

| | Relative position (cM) |
|---|---|
| Marker on BTA9 | http://www.marc.usda.gov/ |
| UWCA9 | 50.0 |
| DIK4720 | 54.0 |
| BMS1290 | 64.9 |
| DIK2816 | 68.1 |
| BM6436 | 77.6 |
| BMS2753 | 79.2 |
| BM4208 | 90.7 |
| BMS2819 | 91.0 |

In a preferred embodiment of the invention, the at least one genetic marker is located in the region from about 50.0 cM to about 79.2 cM (http://www.marc.usda.gov/) on the bovine chromosome BTA9. In one embodiment the at least one genetic marker is located on the bovine chromosome BTA9 in the region flanked by and including the markers UWCA9 and BMS2753. The at least one genetic marker is selected from the group of markers shown in Table 7e:

TABLE 7e

| | Relative position (cM) |
|---|---|
| Marker on BTA9 | http://www.marc.usda.gov/ |
| UWCA9 | 50.0 |
| DIK4720 | 54.0 |
| BMS1290 | 64.9 |
| DIK2816 | 68.1 |
| BM6436 | 77.6 |
| BMS2753 | 79.2 |

In a preferred embodiment of the invention, the at least one genetic marker is located in the region from about 45.7 cM to about 68.1 cM (http://www.marc.usda.gov/) on the bovine chromosome BTA9. In one embodiment the at least one genetic marker is located on the bovine chromosome BTA9 in the region flanked by and including the markers DIK5364 and DIK2816. The at least one genetic marker is selected from the group of markers shown in Table 7f:

TABLE 7f

| | Relative position (cM) |
|---|---|
| Marker on BTA9 | http://www.marc.usda.gov/ |
| DIK5364 | 45.7 |
| UWCA9 | 50.0 |
| DIK4720 | 54.0 |
| BMS1290 | 64.9 |
| DIK2816 | 68.1 |

In a preferred embodiment of the invention, the at least one genetic marker is located in the region from about 12.8 cM to about 43.8 cM (http://www.marc.usda.gov/) on the bovine chromosome BTA9. In one embodiment the at least one genetic marker is located on the bovine chromosome BTA9 in the region flanked by and including the markers ETH225 and DIK5142. The at least one genetic marker is selected from the group of markers shown in Table 7g:

TABLE 7g

| | Relative position (cM) |
|---|---|
| Marker on BTA9 | http://www.marc.usda.gov/ |
| ETH225 | 12.8 |
| ILSTS037 | 26.3 |
| BM2504 | 30.9 |
| DIK2892 | 30.9 |
| DIK3003 | 36.5 |
| DIK3002 | 36.5 |
| BMS1267 | 38.7 |
| DIK5142 | 43.8 |

### BTA10

In one embodiment of the invention the at least one genetic marker is located on the bovine chromosome BTA10. In one specific embodiment of the present invention, the at least one genetic marker is located in the region from about 2.7 cM to about 104.9 cM (http://www.marc.usda.gov/) on the bovine chromosome BTA10. In one embodiment the at least one genetic marker is located on the bovine chromosome BTA10 in the region flanked by and including the markers DIK2658 and BMS2614. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 8a:

TABLE 8a

| | Relative position (cM) |
|---|---|
| Marker on BTA10 | http://www.marc.usda.gov/ |
| DIK2658 | 2.7 |
| DIK2503 | 9.0 |
| CSSM38 | 11.0 |
| BMS528 | 24.0 |
| BM1237 | 24.7 |
| MB077 | 35.1 |
| DIK2000 | 37.5 |
| BMS2742 | 44.3 |
| BMS529 | 55.6 |
| DIK2361 | 56.5 |
| BM888 | 60.0 |
| TGLA433 | 74.0 |
| INRA037 | 79.0 |
| BMS1620 | 80.4 |
| ILSTS070 | 81.4 |
| BMS2641 | 87.5 |
| BMS614 | 100.0 |
| BMS2614 | 109.4 |

In a preferred embodiment of the invention, the at least one genetic marker is located in the region from about 9.0 cM to about 35.1 cM (http://www.marc.usda.gov/) on the bovine chromosome BTA10. In one embodiment the at least one genetic marker is located on the bovine chromosome BTA10 in the region flanked by and including the markers DIK2503 and MB077. The at least one genetic marker is selected from the group of markers shown in Table 8b:

TABLE 8b

| | Relative position (cM) |
|---|---|
| Marker on BTA10 | http://www.marc.usda.gov/ |
| DIK2503 | 9.0 |
| CSSM38 | 11.0 |
| BMS528 | 24.0 |
| BM1237 | 24.7 |
| MB077 | 35.1 |

In a preferred embodiment of the invention, the at least one genetic marker is located in the region from about 11.0 cM to about 37.5 cM (http://www.marc.usda.gov/) on the bovine chromosome BTA10. In one embodiment the at least one genetic marker is located on the bovine chromosome BTA10 in the region flanked by and including the markers CSSM38 and DIK2000. The at least one genetic marker is selected from the group of markers shown in Table 8c:

TABLE 8c

| | Relative position (cM) |
|---|---|
| Marker on BTA10 | http://www.marc.usda.gov/ |
| CSSM38 | 11.0 |
| BMS528 | 24.0 |
| BM1237 | 24.7 |
| MB077 | 35.1 |
| DIK2000 | 37.5 |

In a preferred embodiment of the invention, the at least one genetic marker is located in the region from about 24.0 cM to about 35.1 cM (http://www.marc.usda.gov/) on the bovine chromosome BTA10. In one embodiment the at least one genetic marker is located on the bovine chromosome BTA10 in the region flanked by and including the markers BMS528 and MB077. The at least one genetic marker is selected from the group of markers shown in Table 8d:

TABLE 8d

| | Relative position (cM) |
|---|---|
| Marker on BTA10 | http://www.marc.usda.gov/ |
| BMS528 | 24.0 |
| BM1237 | 24.7 |
| MB077 | 35.1 |

In a preferred embodiment of the invention, the at least one genetic marker is located in the region from about 37.5 cM to about 80.4 cM (http://www.marc.usda.gov/) on the bovine chromosome BTA10. In one embodiment the at least one genetic marker is located on the bovine chromosome BTA10 in the region flanked by and including the markers DIK2000 and BMS1620. The at least one genetic marker is selected from the group of markers shown in Table 8e:

TABLE 8e

| | Relative position (cM) |
|---|---|
| Marker on BTA10 | http://www.marc.usda.gov/ |
| DIK2000 | 37.5 |
| BMS2742 | 44.3 |
| BMS529 | 55.6 |
| DIK2361 | 56.5 |
| BM888 | 60.0 |
| TGLA433 | 74.0 |
| INRA037 | 79.0 |
| BMS1620 | 80.4 |

In a preferred embodiment of the invention, the at least one genetic marker is located in the region from about 44.3 cM to about 74.0 cM (http://www.marc.usda.gov/) on the bovine chromosome BTA10. In one embodiment the at least one genetic marker is located on the bovine chromosome BTA10 in the region flanked by and including the markers BMS2742 and TGLA433. The at least one genetic marker is selected from the group of markers shown in Table 8f:

TABLE 8f

| | Relative position (cM) |
|---|---|
| Marker on BTA10 | http://www.marc.usda.gov/ |
| BMS2742 | 44.3 |
| BMS529 | 55.6 |
| DIK2361 | 56.5 |
| BM888 | 60.0 |
| TGLA433 | 74.0 |

In a preferred embodiment of the invention, the at least one genetic marker is located in the region from about 56.5 cM to about 74.0 cM (http://www.marc.usda.gov/) on the bovine chromosome BTA10. In one embodiment the at least one genetic marker is located on the bovine chromosome BTA10 in the region flanked by and including the markers DIK2361 and TGLA433. The at least one genetic marker is selected from the group of markers shown in Table 8g:

TABLE 8g

| | Relative position (cM) |
|---|---|
| Marker on BTA10 | http://www.marc.usda.gov/ |
| DIK2361 | 56.5 |
| BM888 | 60.0 |
| TGLA433 | 74.0 |

In a preferred embodiment of the invention, the at least one genetic marker is located in the region from about 74.0 cM to about 87.5 cM (http://www.marc.usda.gov/) on the bovine chromosome BTA10. In one embodiment the at least one genetic marker is located on the bovine chromosome BTA10 in the region flanked by and including the markers TGLA433 and BMS2641. The at least one genetic marker is selected from the group of markers shown in Table 8h:

TABLE 8h

| | Relative position (cM) |
|---|---|
| Marker on BTA10 | http://www.marc.usda.gov/ |
| TGLA433 | 74.0 |
| INRA037 | 79.0 |
| BMS1620 | 80.4 |
| ILSTS070 | 81.4 |
| BMS2641 | 87.5 |

In a preferred embodiment of the invention, the at least one genetic marker is located in the region from about 87.5 cM to about 109.4 cM (http://www.marc.usda.gov/) on the bovine chromosome BTA10. In one embodiment the at least one genetic marker is located on the bovine chromosome BTA10 in the region flanked by and including the markers BMS2641 and BMS2614. The at least one genetic marker is selected from the group of markers shown in Table 8i:

TABLE 8i

| | Relative position (cM) |
|---|---|
| Marker on BTA10 | http://www.marc.usda.gov/ |
| BMS2641 | 87.5 |
| BMS614 | 100.0 |
| BMS2614 | 109.4 |

### BTA11

In one embodiment of the invention the at least one genetic marker is located on the bovine chromosome BTA11. In one specific embodiment of the present invention, the at least one genetic marker is located in the region from about 19.4 cM to about 122.4 cM (http://www.marc.usda.gov/) on the bovine chromosome BTA11. In one embodiment the at least one genetic marker is located on the bovine chromosome BTA11 in the region flanked by and including the markers BM716 and HELL 3. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 9a:

TABLE 9a

| | Relative position (cM) |
|---|---|
| Marker on BTA11 | http://www.marc.usda.gov/ |
| BM716 | 19.4 |
| BMS2569 | 21.1 |
| BM2818 | 30.0 |
| INRA177-2 | 34.8 |
| INRA177 | 34.8 |
| RM096 | 40.5 |
| INRA131 | 47.3 |
| BM7169 | 50.3 |
| BM6445 | 61.6 |
| ILSTS036 | 61.6 |
| BMS1822 | 65.9 |
| TGLA58 | 73.1 |
| BMS2047 | 78.5 |
| HUJV174 | 92.2 |
| BMS989 | 92.2 |
| TGLA436 | 105.2 |
| BMS460 | 109.4 |
| ILSTS045 | 114.2 |
| DIK4819 | 115.0 |
| HEL13 | 122.4 |

In a preferred embodiment of the invention, the at least one genetic marker is located in the region from about 19.4 cM to about 92.2 cM (http://www.marc.usda.gov/) on the bovine chromosome BTA11. In one embodiment the at least one genetic marker is located on the bovine chromosome BTA11 in the region flanked by and including the markers BM716 and BMS989. The at least one genetic marker is selected from the group of markers shown in Table 9b:

TABLE 9b

| | Relative position (cM) |
|---|---|
| Marker on BTA11 | http://www.marc.usda.gov/ |
| BM716 | 19.4 |
| BMS2569 | 21.1 |
| BM2818 | 30.0 |
| INRA177-2 | 34.8 |
| INRA177 | 34.8 |
| RM096 | 40.5 |
| INRA131 | 47.3 |
| BM7169 | 50.3 |
| BM6445 | 61.6 |
| ILSTS036 | 61.6 |
| BMS1822 | 65.9 |
| TGLA58 | 73.1 |
| BMS2047 | 78.5 |
| HUJV174 | 92.2 |
| BMS989 | 92.2 |

In a preferred embodiment of the invention, the at least one genetic marker is located in the region from about 19.4 cM to about 50.3 cM (http://www.marc.usda.gov/) on the bovine chromosome BTA11. In one embodiment the at least one genetic marker is located on the bovine chromosome BTA11 in the region flanked by and including the markers BM716 and BM7169. The at least one genetic marker is selected from the group of markers shown in Table 9c:

TABLE 9c

| | Relative position (cM) |
|---|---|
| Marker on BTA11 | http://www.marc.usda.gov/ |
| BM716 | 19.4 |
| BMS2569 | 21.1 |
| BM2818 | 30.0 |
| INRA177-2 | 34.8 |
| INRA177 | 34.8 |
| RM096 | 40.5 |
| INRA131 | 47.3 |
| BM7169 | 50.3 |

In a preferred embodiment of the invention, the at least one genetic marker is located in the region from about 30.0 cM to about 50.3 cM (http://www.marc.usda.gov/) on the bovine chromosome BTA11. In one embodiment the at least one genetic marker is located on the bovine chromosome BTA11 in the region flanked by and including the markers BM2818 and BM7169. The at least one genetic marker is selected from the group of markers shown in Table 9d:

TABLE 9d

| | Relative position (cM) |
|---|---|
| Marker on BTA11 | http://www.marc.usda.gov/ |
| BM2818 | 30.0 |
| INRA177-2 | 34.8 |
| INRA177 | 34.8 |
| RM096 | 40.5 |
| INRA131 | 47.3 |
| BM7169 | 50.3 |

In a preferred embodiment of the invention, the at least one genetic marker is located in the region from about 34.8 cM to about 47.3 cM (http://www.marc.usda.gov/) on the bovine chromosome BTA11. In one embodiment the at least one genetic marker is located on the bovine chromosome BTA11 in the region flanked by and including the markers INRA177-2 and INRA131. The at least one genetic marker is selected from the group of markers shown in Table 9e:

TABLE 9e

| | Relative position (cM) |
|---|---|
| Marker on BTA11 | http://www.marc.usda.gov/ |
| INRA177-2 | 34.8 |
| INRA177 | 34.8 |
| RM096 | 40.5 |
| INRA131 | 47.3 |

In a preferred embodiment of the invention, the at least one genetic marker is located in the region from about 50.3 cM to about 92.2 cM (http://www.marc.usda.gov/) on the bovine chromosome BTA11. In one embodiment the at least one genetic marker is located on the bovine chromosome BTA11 in the region flanked by and including the markers BM7169 and BMS989. The at least one genetic marker is selected from the group of markers shown in Table 9f:

TABLE 9f

| | Relative position (cM) |
|---|---|
| Marker on BTA11 | http://www.marc.usda.gov/ |
| BM7169 | 50.3 |
| BM6445 | 61.6 |
| ILSTS036 | 61.6 |
| BMS1822 | 65.9 |
| TGLA58 | 73.1 |
| BMS2047 | 78.5 |
| HUJV174 | 92.2 |
| BMS989 | 92.2 |

In a preferred embodiment of the invention, the at least one genetic marker is located in the region from about 61.6 cM to about 92.2 cM (http://www.marc.usda.gov/) on the bovine chromosome BTA11. In one embodiment the at least one genetic marker is located on the bovine chromosome BTA11 in the region flanked by and including the markers BM6445 and BMS989. The at least one genetic marker is selected from the group of markers shown in Table 9g:

TABLE 9g

| | Relative position (cM) |
|---|---|
| Marker on BTA11 | http://www.marc.usda.gov/ |
| BM6445 | 61.6 |
| ILSTS036 | 61.6 |
| BMS1822 | 65.9 |
| TGLA58 | 73.1 |
| BMS2047 | 78.5 |
| HUJV174 | 92.2 |
| BMS989 | 92.2 |

In a preferred embodiment of the invention, the at least one genetic marker is located in the region from about 73.3 cM to about 92.2 cM (http://www.marc.usda.gov/) on the bovine chromosome BTA11. In one embodiment the at least one genetic marker is located on the bovine chromosome BTA11 in the region flanked by and including the markers TGLA58 and BMS989. The at least one genetic marker is selected from the group of markers shown in Table 9h:

TABLE 9h

| | Relative position (cM) |
|---|---|
| Marker on BTA11 | http://www.marc.usda.gov/ |
| TGLA58 | 73.1 |
| BMS2047 | 78.5 |
| HUJV174 | 92.2 |
| BMS989 | 92.2 |

In a preferred embodiment of the invention, the at least one genetic marker is located in the region from about 92.2 cM to about 109.4 cM (http://www.marc.usda.gov/) on the bovine chromosome BTA1. In one embodiment the at least one genetic marker is located on the bovine chromosome BTA11 in the region flanked by and including the markers HUJV174 and BMS460. The at least one genetic marker is selected from the group of markers shown in Table 9i:

TABLE 9i

| | Relative position (cM) |
|---|---|
| Marker on BTA11 | http://www.marc.usda.gov/ |
| HUJV174 | 92.2 |
| BMS989 | 92.2 |
| TGLA436 | 105.2 |
| BMS460 | 109.4 |

### BTA12

In one embodiment of the invention the at least one genetic marker is located on the bovine chromosome BTA12. In one specific embodiment of the present invention, the at least one genetic marker is located in the region from about 0.0 cM to about 109.0 cM (http://www.marc.usda.gov/) on the bovine chromosome BTA12. In one embodiment the at least one genetic marker is located on the bovine chromosome BTA12 in the region flanked by and including the markers BMS410 and BMS2724. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 10a:

TABLE 10a

| | Relative position (cM) |
|---|---|
| Marker on BTA12 | http://www.marc.usda.gov/ |
| BMS410 | 0.0 |
| BM6108 | 15.1 |
| BM860 | 50.4 |
| BM3975 | 63.8 |
| BMS1316 | 102.0 |
| BMS2724 | 109.0 |

In a preferred embodiment of the invention, the at least one genetic marker is located in the region from about 50.4 cM to about 109.0 cM (http://www.marc.usda.gov/) on the bovine chromosome BTA12. In one embodiment the at least one genetic marker is located on the bovine chromosome BTA12 in the region flanked by and including the markers BM860 and BMS2724. The at least one genetic marker is selected from the group of markers shown in Table 10b:

TABLE 10b

| | Relative position (cM) |
|---|---|
| Marker on BTA12 | http://www.marc.usda.gov/ |
| BM860 | 50.4 |
| BMS975 | 63.8 |
| BMS1316 | 102.0 |
| BMS2724 | 109.0 |

In a preferred embodiment of the invention, the at least one genetic marker is located in the region from about 50.4 cM to about 102.0 cM (http://www.marc.usda.gov/) on the bovine chromosome BTA12. In one embodiment the at least one genetic marker is located on the bovine chromosome BTA12 in the region flanked by and including the markers BM860 and BMS1316. The at least one genetic marker is selected from the group of markers shown in Table 10c:

TABLE 10c

| | Relative position (cM) |
|---|---|
| Marker on BTA12 | http://www.marc.usda.gov/ |
| BM860 | 50.4 |
| BMS975 | 63.8 |
| BMS1316 | 102.0 |

In a preferred embodiment of the invention, the at least one genetic marker is located in the region from about 63.8 cM to about 102.0 cM (http://www.marc.usda.gov/) on the bovine chromosome BTA12. In one embodiment the at least one genetic marker is located on the bovine chromosome BTA12 in the region flanked by and including the markers BMS975 and BMS1316. The at least one genetic marker is selected from the group of markers shown in Table 10d:

TABLE 10d

| | Relative position (cM) |
|---|---|
| Marker on BTA12 | http://www.marc.usda.gov/ |
| BMS975 | 63.8 |
| BMS1316 | 102.0 |

### BTA15

In one embodiment of the invention the at least one genetic marker is located on the bovine chromosome BTA15. In one specific embodiment of the present invention, the at least one genetic marker is located in the region from about 9.4 cM to about 109.8 cM (http://www.marc.usda.gov/) on the bovine chromosome BTA15. In one embodiment the at least one genetic marker is located on the bovine chromosome BTA15 in the region flanked by and including the markers BR3510 and BMS429. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 11a:

TABLE 11a

| | Relative position (cM) |
|---|---|
| Marker on BTA15 | http://www.marc.usda.gov/ |
| BR3510 | 9.4 |
| BMS2533 | 13.9 |
| INRA050 | 31.1 |
| JAB8 | 31.2 |
| BMS2684 | 48.2 |
| DIK1106 | 51.9 |
| INRA145 | 67.8 |
| IDVGA-10 | 67.8 |
| DIK4850 | 74.1 |
| DIK2768 | 77.9 |
| ILSTS027 | 83.4 |
| BMS812 | 84.9 |
| BMS2076 | 91.8 |
| BL1095 | 94.8 |
| BMS820 | 98.2 |
| BMS927 | 105.0 |
| BMS429 | 109.8 |

In a preferred embodiment of the invention, the at least one genetic marker is located in the region from about 48.2 cM to about 109.8 cM (http://www.marc.usda.gov/) on the bovine chromosome BTA15. In one embodiment the at least one genetic marker is located on the bovine chromosome BTA15 in the region flanked by and including the markers BMS2684 and BMS429. The at least one genetic marker is selected from the group of markers shown in Table 11b:

TABLE 11b

| | Relative position (cM) |
|---|---|
| Marker on BTA15 | http://www.marc.usda.gov/ |
| BMS2684 | 48.2 |
| DIK1106 | 51.9 |
| INRA145 | 67.8 |
| IDVGA-10 | 67.8 |
| DIK4850 | 74.1 |
| DIK2768 | 77.9 |
| ILSTS027 | 83.4 |
| BMS812 | 84.9 |
| BMS2076 | 91.8 |
| BL1095 | 94.8 |
| BMS820 | 98.2 |
| BMS927 | 105.0 |
| BMS429 | 109.8 |

In a preferred embodiment of the invention, the at least one genetic marker is located in the region from about 48.2 cM to about 91.8 cM (http://www.marc.usda.gov/) on the bovine chromosome BTA15. In one embodiment the at least one genetic marker is located on the bovine chromosome BTA15 in the region flanked by and including the markers BMS2684 and BMS2076. The at least one genetic marker is selected from the group of markers shown in Table 11c:

TABLE 11c

| | Relative position (cM) |
|---|---|
| Marker on BTA15 | http://www.marc.usda.gov/ |
| BMS2684 | 48.2 |
| DIK1106 | 51.9 |
| INRA145 | 67.8 |
| IDVGA-10 | 67.8 |
| DIK4850 | 74.1 |
| DIK2768 | 77.9 |
| ILSTS027 | 83.4 |
| BMS812 | 84.9 |
| BMS2076 | 91.8 |

In a preferred embodiment of the invention, the at least one genetic marker is located in the region from about 77.9 cM to about 109.8 cM (http://www.marc.usda.gov/) on the bovine chromosome BTA15. In one embodiment the at least one genetic marker is located on the bovine chromosome BTA15 in the region flanked by and including the markers 77.9 and 109.8. The at least one genetic marker is selected from the group of markers shown in Table 11d:

TABLE 11d

| | Relative position (cM) |
|---|---|
| Marker on BTA15 | http://www.marc.usda.gov/ |
| DIK2768 | 77.9 |
| ILSTS027 | 83.4 |
| BMS812 | 84.9 |
| BMS2076 | 91.8 |
| BL1095 | 94.8 |
| BMS820 | 98.2 |
| BMS927 | 105.0 |
| BMS429 | 109.8 |

In a preferred embodiment of the invention, the at least one genetic marker is located in the region from about 84.9 cM to about 109.8 cM (http://www.marc.usda.gov/) on the bovine chromosome BTA15. In one embodiment the at least one genetic marker is located on the bovine chromosome BTA15 in the region flanked by and including the markers BMS812 and BMS429. The at least one genetic marker is selected from the group of markers shown in Table 11e:

TABLE 11e

| | Relative position (cM) |
|---|---|
| Marker on BTA15 | http://www.marc.usda.gov/ |
| BMS812 | 84.9 |
| BMS2076 | 91.8 |
| BL1095 | 94.8 |
| BMS820 | 98.2 |
| BMS927 | 105.0 |
| BMS429 | 109.8 |

In a preferred embodiment of the invention, the at least one genetic marker is located in the region from about 84.9 cM to about 94.8 cM (http://www.marc.usda.gov/) on the bovine chromosome BTA15. In one embodiment the at least one genetic marker is located on the bovine chromosome BTA15 in the region flanked by and including the markers BMS812 and BL1095. The at least one genetic marker is selected from the group of markers shown in Table 11f:

TABLE 11f

| | Relative position (cM) |
|---|---|
| Marker on BTA15 | http://www.marc.usda.gov/ |
| BMS812 | 84.9 |
| BMS2076 | 91.8 |
| BL1095 | 94.8 |

In a preferred embodiment of the invention, the at least one genetic marker is located in the region from about 91.8 cM to about 105.0 cM (http://www.marc.usda.gov/) on the bovine chromosome BTA15. In one embodiment the at least one genetic marker is located on the bovine chromosome BTA15 in the region flanked by and including the markers BMS2076 and BMS927. The at least one genetic marker is selected from the group of markers shown in Table 11g:

TABLE 11g

| | Relative position (cM) |
|---|---|
| Marker on BTA15 | http://www.marc.usda.gov/ |
| BMS2076 | 91.8 |
| BL1095 | 94.8 |
| BMS820 | 98.2 |
| BMS927 | 105.0 |

In a preferred embodiment of the invention, the at least one genetic marker is located in the region from about 98.2 cM to about 109.8 cM (http://www.marc.usda.gov/) on the bovine chromosome BTA15. In one embodiment the at least one genetic marker is located on the bovine chromosome BTA15 in the region flanked by and including the markers BMS820 and BMS429. The at least one genetic marker is selected from the group of markers shown in Table 11 h:

TABLE 11h

| | Relative position (cM) |
|---|---|
| Marker on BTA15 | http://www.marc.usda.gov/ |
| BMS820 | 98.2 |
| BMS927 | 105.0 |
| BMS429 | 109.8 |

In a preferred embodiment of the invention, the at least one genetic marker is located in the region from about 105.0 cM to about 109.8 cM (http://www.marc.usda.gov/) on the bovine chromosome BTA15. In one embodiment the at least one genetic marker is located on the bovine chromosome BTA15 in the region flanked by and including the markers BMS927 and BMS429. The at least one genetic marker is selected from the group of markers shown in Table 11i:

TABLE 11i

| | Relative position (cM) |
|---|---|
| Marker on BTA15 | http://www.marc.usda.gov/ |
| BMS927 | 105.0 |
| BMS429 | 109.8 |

### BTA18

In one embodiment of the invention the at least one genetic marker is located on the bovine chromosome BTA18. In one specific embodiment of the present invention, the at least one genetic marker is located in the region from about 0.0 cM to about 84.4 cM (http://www.marc.usda.gov/) on the bovine chromosome BTA18. In one embodiment the at least one genetic marker is located on the bovine chromosome BTA18 in the region flanked by and including the markers IDVGA-31 and DIK4013. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 12a:

TABLE 12a

| | Relative position (cM) |
|---|---|
| Marker on BTA18 | http://www.marc.usda.gov/ |
| IDVGA-31 | 0.0 |
| BMS1355 | 2.9 |
| BMS1322 | 13.5 |
| TEXAN-10 | 20.7 |
| BMS2213 | 24.5 |
| INRA121 | 30.2 |
| BR4406 | 33.4 |
| BMS2554 | 40.2 |
| MNB-27 | 44.0 |
| BM7109 | 47.0 |
| INRA063 | 48.0 |
| ILSTS002 | 54.7 |
| BMS2639 | 55.5 |
| DIK4960 | 56.3 |
| DIK4849 | 57.0 |
| BMON117 | 57.6 |
| DIK4232 | 61.2 |
| BMS2785 | 72.0 |
| DIK4569 | 73.8 |
| BM2078 | 76.8 |
| BM6507 | 78.8 |
| TGLA227 | 84.1 |
| DIK4013 | 84.4 |

In a preferred embodiment of the invention, the at least one genetic marker is located in the region from about 0.0 cM to about 13.5 cM (http://www.marc.usda.gov/) on the bovine chromosome BTA18. In one embodiment the at least one genetic marker is located on the bovine chromosome BTA18 in the region flanked by and including the markers IDVGA-31 and BMS1322. The at least one genetic marker is selected from the group of markers shown in Table 12b:

TABLE 12b

| | Relative position (cM) |
|---|---|
| Marker on BTA18 | http://www.marc.usda.gov/ |
| IDVGA-31 | 0.0 |
| BMS1355 | 2.9 |
| BMS1322 | 13.5 |

In a preferred embodiment of the invention, the at least one genetic marker is located in the region from about 2.9 cM to about 13.5 cM (http://www.marc.usda.gov/) on the bovine chromosome BTA18. In one embodiment the at least one genetic marker is located on the bovine chromosome BTA18 in the region flanked by and including the markers BMS1355 and BMS1322. The at least one genetic marker is selected from the group of markers shown in Table 12c:

TABLE 12c

| | Relative position (cM) |
|---|---|
| Marker on BTA18 | http://www.marc.usda.gov/ |
| BMS1355 | 2.9 |
| BMS1322 | 13.5 |

In a preferred embodiment of the invention, the at least one genetic marker is located 10 in the region from about 30.2 cM to about 61.2 cM (http://www.marc.usda.gov/) on the bovine chromosome BTA18. In one embodiment the at least one genetic marker is located on the bovine chromosome BTA18 in the region flanked by and including the markers INRA121 and DIK4232. The at least one genetic marker is selected from the group of markers shown in Table 12d:

TABLE 12d

| | Relative position (cM) |
|---|---|
| Marker on BTA18 | http://www.marc.usda.gov/ |
| INRA121 | 30.2 |
| BR4406 | 33.4 |
| BMS2554 | 40.2 |
| MNB-27 | 44.0 |
| BM7109 | 47.0 |
| INRA063 | 48.0 |
| ILSTS002 | 54.7 |
| BMS2639 | 55.5 |
| DIK4960 | 56.3 |
| DIK4849 | 57.0 |
| BMON117 | 57.6 |
| DIK4232 | 61.2 |

In a preferred embodiment of the invention, the at least one genetic marker is located in the region from about 33.4 cM to about 54.7 cM (http://www.marc.usda.gov/) on the bovine chromosome BTA18. In one embodiment the at least one genetic marker is located on the bovine chromosome BTA18 in the region flanked by and including the markers BR4406 and ILSTS002. The at least one genetic marker is selected from the group of markers shown in Table 12e:

TABLE 12e

| | Relative position (cM) |
|---|---|
| Marker on BTA18 | http://www.marc.usda.gov/ |
| BR4406 | 33.4 |
| BMS2554 | 40.2 |
| MNB-27 | 44.0 |
| BM7109 | 47.0 |
| INRA063 | 48.0 |
| ILSTS002 | 54.7 |

In a preferred embodiment of the invention, the at least one genetic marker is located in the region from about 57.6 cM to about 84.4 cM (http://www.marc.usda.gov/) on the bovine chromosome BTA18. In one embodiment the at least one genetic marker is located on the bovine chromosome BTA18 in the region flanked by and including the markers BMON117 and DIK4013. The at least one genetic marker is selected from the group of markers shown in Table 12f:

TABLE 12f

| | Relative position (cM) |
|---|---|
| Marker on BTA18 | http://www.marc.usda.gov/ |
| BMON117 | 57.6 |
| DIK4232 | 61.2 |
| BMS2785 | 72.0 |
| DIK4569 | 73.8 |
| BM2078 | 76.8 |
| BM6507 | 78.8 |
| TGLA227 | 84.1 |
| DIK4013 | 84.4 |

In a preferred embodiment of the invention, the at least one genetic marker is located in the region from about 61.2 cM to about 84.4 cM (http://www.marc.usda.gov/) on the bovine chromosome BTA18. In one embodiment the at least one genetic marker is located on the bovine chromosome BTA18 in the region flanked by and including the markers DIK4232 and DIK4013. The at least one genetic marker is selected from the group of markers shown in Table 12g:

TABLE 12g

| | Relative position (cM) |
|---|---|
| Marker on BTA18 | http://www.marc.usda.gov/ |
| DIK4232 | 61.2 |
| BMS2785 | 72.0 |
| DIK4569 | 73.8 |
| BM2078 | 76.8 |
| BM6507 | 78.8 |
| TGLA227 | 84.1 |
| DIK4013 | 84.4 |

In a preferred embodiment of the invention, the at least one genetic marker is located in the region from about 72.0 cM to about 76.8 cM (http://www.marc.usda.gov/) on the bovine chromosome BTA18. In one embodiment the at least one genetic marker is located on the bovine chromosome BTA18 in the region flanked by and including the markers BMS2785 and BM2078. The at least one genetic marker is selected from the group of markers shown in Table 12h:

TABLE 12h

| | Relative position (cM) |
|---|---|
| Marker on BTA18 | http://www.marc.usda.gov/ |
| BMS2785 | 72.0 |
| DIK4569 | 73.8 |
| BM2078 | 76.8 |

In a preferred embodiment of the invention, the at least one genetic marker is located in the region from about 76.8 cM to about 84.4 cM (http://www.marc.usda.gov/) on the bovine chromosome BTA18. In one embodiment the at least one genetic marker is located on the bovine chromosome BTA18 in the region flanked by and including the markers BM2078 and DIK4013. The at least one genetic marker is selected from the group of markers shown in Table 12i:

TABLE 12i

| | Relative position (cM) |
|---|---|
| Marker on BTA18 | http://www.marc.usda.gov/ |
| BM2078 | 76.8 |
| BM6507 | 78.8 |
| TGLA227 | 84.1 |
| DIK4013 | 84.4 |

In a preferred embodiment of the invention, the at least one genetic marker is located in the region from about 76.8 cM to about 78.8 cM (http://www.marc.usda.gov/) on the bovine chromosome BTA18. In one embodiment the at least one genetic marker is located on the bovine chromosome BTA18 in the region flanked by and including the markers BM2078 and BM6507. The at least one genetic marker is selected from the group of markers shown in Table 12j:

TABLE 12j

| | Relative position (cM) |
|---|---|
| Marker on BTA18 | http://www.marc.usda.gov/ |
| BM2078 | 76.8 |
| BM6507 | 78.8 |

In a preferred embodiment of the invention, the at least one genetic marker is located in the region from about 78.8 cM to about 84.4 cM (http://www.marc.usda.gov/) on the bovine chromosome BTA18. In one embodiment the at least one genetic marker is located on the bovine chromosome BTA18 in the region flanked by and including the markers BM6507 and DIK4013. The at least one genetic marker is selected from the group of markers shown in Table 12k:

TABLE 12k

| | Relative position (cM) |
|---|---|
| Marker on BTA18 | http://www.marc.usda.gov/ |
| BM6507 | 78.8 |
| TGLA227 | 84.1 |
| DIK4013 | 84.4 |

### BTA19

In one embodiment of the invention the at least one genetic marker is located on the bovine chromosome BTA19. In one specific embodiment of the present invention, the at least one genetic marker is located in the region from about 0.0 cM to about 108.0 cM (http://www.marc.usda.gov/) on the bovine chromosome BTA19. In one embodiment the at least one genetic marker is located on the bovine chromosome BTA19 in the region flanked by and including the markers BM9202 and BMS601. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 13a:

TABLE 13a

| | Relative position (cM) |
|---|---|
| Marker on BTA19 | http://www.marc.usda.gov/ |
| BM9202 | 0.0 |
| BMS745 | 16.0 |
| BP20 | 45.9 |
| IDVGA-46 | 47.0 |
| BMS2389 | 52.2 |
| CSSM065 | 69.8 |
| ETH3 | 90.0 |
| BMS601 | 108.0 |

In a preferred embodiment of the invention, the at least one genetic marker is located in the region from about 0.0 cM to about 90.0 cM (http://www.marc.usda.gov/) on the bovine chromosome BTA19. In one embodiment the at least one genetic marker is located on the bovine chromosome BTA19 in the region flanked by and including the markers BM9202 and ETH3. The at least one genetic marker is selected from the group of markers shown in Table 13b:

TABLE 13b

| | Relative position (cM) |
|---|---|
| Marker on BTA19 | http://www.marc.usda.gov/ |
| BM9202 | 0.0 |
| BMS745 | 16.0 |
| BP20 | 45.9 |
| IDVGA-46 | 47.0 |
| BMS2389 | 52.2 |
| CSSM065 | 69.8 |
| ETH3 | 90.0 |

In a preferred embodiment of the invention, the at least one genetic marker is located in the region from about 0.0 cM to about 45.9 cM (http://www.marc.usda.gov/) on the bovine chromosome BTA19. In one embodiment the at least one genetic marker is located on the bovine chromosome BTA19 in the region flanked by and including the markers BM9202 and BP20. The at least one genetic marker is selected from the group of markers shown in Table 13c:

TABLE 13c

| | Relative position (cM) |
|---|---|
| Marker on BTA19 | http://www.marc.usda.gov/ |
| BM9202 | 0.0 |
| BMS745 | 16.0 |
| BP20 | 45.9 |

In a preferred embodiment of the invention, the at least one genetic marker is located in the region from about 16.0 cM to about 45.9 cM (http://www.marc.usda.gov/) on the bovine chromosome BTA19. In one embodiment the at least one genetic marker is located on the bovine chromosome BTA19 in the region flanked by and including the markers BMS745 and BP20. The at least one genetic marker is selected from the group of markers shown in Table 13d:

TABLE 13d

| | Relative position (cM) |
|---|---|
| Marker on BTA19 | http://www.marc.usda.gov/ |
| BMS745 | 16.0 |
| BP20 | 45.9 |

In a preferred embodiment of the invention, the at least one genetic marker is located in the region from about 47.0 cM to about 90.0 cM (http://www.marc.usda.gov/) on the bovine chromosome BTA19. In one embodiment the at least one genetic marker is located on the bovine chromosome BTA19 in the region flanked by and including the markers IDVGA-46 and ETH3. The at least one genetic marker is selected from the group of markers shown in Table 13e:

TABLE 13e

| | Relative position (cM) |
|---|---|
| Marker on BTA19 | http://www.marc.usda.gov/ |
| IDVGA-46 | 47.0 |
| BMS2389 | 52.2 |
| CSSM065 | 69.8 |
| ETH3 | 90.0 |

In a preferred embodiment of the invention, the at least one genetic marker is located in the region from about 52.2 cM to about 108.0 cM (http://www.marc.usda.gov/) on the bovine chromosome BTA19. In one embodiment the at least one genetic marker is located on the bovine chromosome BTA19 in the region flanked by and including the markers BMS2389 and BMS601. The at least one genetic marker is selected from the group of markers shown in Table 13f:

TABLE 13f

| | Relative position (cM) |
|---|---|
| Marker on BTA19 | http://www.marc.usda.gov/ |
| BMS2389 | 52.2 |
| CSSM065 | 69.8 |
| ETH3 | 90.0 |
| BMS601 | 108.0 |

In a preferred embodiment of the invention, the at least one genetic marker is located in the region from about 69.8 cM to about 90.0 cM (http://www.marc.usda.gov/) on the bovine chromosome BTA19. In one embodiment the at least one genetic marker is located on the bovine chromosome BTA19 in the region flanked by and including the markers CSSM065 and ETH3. The at least one genetic marker is selected from the group of markers shown in Table 13g:

TABLE 13g

| | Relative position (cM) |
|---|---|
| Marker on BTA19 | http://www.marc.usda.gov/ |
| CSSM065 | 69.8 |
| ETH3 | 90.0 |

### BTA20

In one embodiment of the invention the at least one genetic marker is located on the bovine chromosome BTA20. In one specific embodiment of the present invention, the at least one genetic marker is located in the region from about 0.0 cM to about 77.1 cM (http://www.marc.usda.gov/) on the bovine chromosome BTA20. In one embodiment the at least one genetic marker is located on the bovine chromosome BTA20 in the region flanked by and including the markers BM3517 and UWCA26. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 14a:

TABLE 14a

| | Relative position (cM) |
|---|---|
| Marker on BTA20 | http://www.marc.usda.gov/ |
| BM3517 | 0.0 |
| HEL12 | 0.6 |
| BMS1282 | 19.1 |
| BMS1754 | 26.3 |
| TGLA126 | 31.9 |
| BMS2361 | 49.7 |
| AGLA29 | 55.1 |
| BMS703 | 60.1 |
| BM5004 | 71.8 |
| UWCA26 | 77.1 |

In a preferred embodiment of the invention, the at least one genetic marker is located in the region from about 0.0 cM to about 71.8 cM (http://www.marc.usda.gov/) on the bovine chromosome BTA20. In one embodiment the at least one genetic marker is located on the bovine chromosome BTA20 in the region flanked by and including the markers BM3517 and BM5004. The at least one genetic marker is selected from the group of markers shown in Table 14b:

TABLE 14b

| | Relative position (cM) |
|---|---|
| Marker on BTA20 | http://www.marc.usda.gov/ |
| BM3517 | 0.0 |
| HEL12 | 0.6 |
| BMS1282 | 19.1 |
| BMS1754 | 26.3 |
| TGLA126 | 31.9 |
| BMS2361 | 49.7 |
| AGLA29 | 55.1 |
| BMS703 | 60.1 |
| BM5004 | 71.8 |

In a preferred embodiment of the invention, the at least one genetic marker is located in the region from about 0.0 cM to about 26.3 cM (http://www.marc.usda.gov/) on the bovine chromosome BTA20. In one embodiment the at least one genetic marker is located on the bovine chromosome BTA20 in the region flanked by and including the markers BM3517 and BMS1754. The at least one genetic marker is selected from the group of markers shown in Table 14c:

TABLE 14c

| | Relative position (cM) |
|---|---|
| Marker on BTA20 | http://www.marc.usda.gov/ |
| BM3517 | 0.0 |
| HEL12 | 0.6 |
| BMS1282 | 19.1 |
| BMS1754 | 26.3 |

In a preferred embodiment of the invention, the at least one genetic marker is located in the region from about 0.6 cM to about 19.1 cM (http://www.marc.usda.gov/) on the bovine chromosome BTA20. In one embodiment the at least one genetic marker is located on the bovine chromosome BTA20 in the region flanked by and including the markers HEL12 and BMS1282. The at least one genetic marker is selected from the group of markers shown in Table 14d:

TABLE 14d

| | Relative position (cM) |
|---|---|
| Marker on BTA20 | http://www.marc.usda.gov/ |
| HEL12 | 0.6 |
| BMS1282 | 19.1 |

In a preferred embodiment of the invention, the at least one genetic marker is located in the region from about 19.1 cM to about 55.1 cM (http://www.marc.usda.gov/) on the bovine chromosome BTA20. In one embodiment the at least one genetic marker is located on the bovine chromosome BTA20 in the region flanked by and including the markers BMS1282 and AGLA29. The at least one genetic marker is selected from the group of markers shown in Table 14e:

TABLE 14e

| | Relative position (cM) |
|---|---|
| Marker on BTA20 | http://www.marc.usda.gov/ |
| BMS1282 | 19.1 |
| BMS1754 | 26.3 |
| TGLA126 | 31.9 |
| BMS2361 | 49.7 |
| AGLA29 | 55.1 |

In a preferred embodiment of the invention, the at least one genetic marker is located in the region from about 31.9 cM to about 49.7 cM (http://www.marc.usda.gov/) on the bovine chromosome BTA20. In one embodiment the at least one genetic marker is located on the bovine chromosome BTA20 in the region flanked by and including the markers TGLA126 and BMS2361. The at least one genetic marker is selected from the group of markers shown in Table 14f:

TABLE 14f

| | Relative position (cM) |
|---|---|
| Marker on BTA20 | http://www.marc.usda.gov/ |
| TGLA126 | 31.9 |
| BMS2361 | 49.7 |

In a preferred embodiment of the invention, the at least one genetic marker is located in the region from about 49.7 cM to about 55.1 cM (http://www.marc.usda.gov/) on the bovine chromosome BTA20. In one embodiment the at least one genetic marker is located on the bovine chromosome BTA20 in the region flanked by and including the markers BMS2361 and AGLA29. The at least one genetic marker is selected from the group of markers shown in Table 14g:

TABLE 14g

| | Relative position (cM) |
|---|---|
| Marker on BTA20 | http://www.marc.usda.gov/ |
| BMS2361 | 49.7 |
| AGLA29 | 55.1 |

In a preferred embodiment of the invention, the at least one genetic marker is located in the region from about 55.1 cM to about 77.1 cM (http://www.marc.usda.gov/) on the bovine chromosome BTA20. In one embodiment the at least one genetic marker is located on the bovine chromosome BTA20 in the region flanked by and including the markers AGLA29 and UWCA26. The at least one genetic marker is selected from the group of markers shown in Table 14h:

TABLE 14h

| | Relative position (cM) |
|---|---|
| Marker on BTA20 | http://www.marc.usda.gov/ |
| AGLA29 | 55.1 |
| BMS703 | 60.1 |
| BM5004 | 71.8 |
| UWCA26 | 77.1 |

In a preferred embodiment of the invention, the at least one genetic marker is located in the region from about 60.1 cM to about 71.8 cM (http://www.marc.usda.gov/) on the bovine chromosome BTA20. In one embodiment the at least one genetic marker is located on the bovine chromosome BTA20 in the region flanked by and including the markers BMS703 and BM5004. The at least one genetic marker is selected from the group of markers shown in Table 14i:

TABLE 14i

| | Relative position (cM) |
|---|---|
| Marker on BTA20 | http://www.marc.usda.gov/ |
| BMS703 | 60.1 |
| BM5004 | 71.8 |

### BTA21

In one embodiment of the invention the at least one genetic marker is located on the bovine chromosome BTA21. In one specific embodiment of the present invention, the at least one genetic marker is located in the region from about 5.6 cM to about 76.8 cM (http://www.marc.usda.gov/) on the bovine chromosome BTA21. In one embodiment the at least one genetic marker is located on the bovine chromosome BTA21 in the region flanked by and including the markers DIK5182 and IDVGA-30. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 15a:

TABLE 15a

| | Relative position (cM) |
|---|---|
| Marker on BTA21 | http://www.marc.usda.gov/ |
| DIK5182 | 5.5 |
| BMS1117 | 11.0 |
| RM151 | 12.6 |
| DIK2492 | 18.3 |
| AGLA233 | 21.2 |
| ILSTS095 | 23.7 |
| DIK4602 | 24.3 |
| BM103 | 29.8 |
| DIK4001 | 30.0 |
| IDVGA-45 | 30.9 |
| DIK2481 | 33.7 |
| INRA103 | 35.9 |
| BMS2815 | 41.7 |
| DIK2842 | 41.7 |
| DIK3036 | 47.8 |
| DIK4391 | 52.1 |
| DIK2913 | 57.1 |
| BM846 | 61.2 |
| TGLA122 | 62.7 |
| ILSTS054 | 65.8 |
| BMS743 | 75.3 |
| IDVGA-30 | 76.8 |

In a preferred embodiment of the invention, the at least one genetic marker is located in the region from about 11.0 cM to about 61.2 cM (http://www.marc.usda.gov/) on the bovine chromosome BTA21. In one embodiment the at least one genetic marker is located on the bovine chromosome BTA21 in the region flanked by and including the markers BMS1117 and BM846. The at least one genetic marker is selected from the group of markers shown in Table 15b:

TABLE 15b

| | Relative position (cM) |
|---|---|
| Marker on BTA21 | http://www.marc.usda.gov/ |
| BMS1117 | 11.0 |
| RM151 | 12.6 |
| DIK2492 | 18.3 |
| AGLA233 | 21.2 |
| ILSTS095 | 23.7 |
| DIK4602 | 24.3 |
| BM103 | 29.8 |
| DIK4001 | 30.0 |
| IDVGA-45 | 30.9 |
| DIK2481 | 33.7 |
| INRA103 | 35.9 |
| BMS2815 | 41.7 |
| DIK2842 | 41.7 |
| DIK3036 | 47.8 |
| DIK4391 | 52.1 |
| DIK2913 | 57.1 |
| BM846 | 61.2 |

In a preferred embodiment of the invention, the at least one genetic marker is located in the region from about 18.3 cM to about 57.1 cM (http://www.marc.usda.gov/) on the bovine chromosome BTA21. In one embodiment the at least one genetic marker is located on the bovine chromosome BTA21 in the region flanked by and including the markers DIK2492 and DIK2913. The at least one genetic marker is selected from the group of markers shown in Table 15c:

TABLE 15c

| | Relative position (cM) |
|---|---|
| Marker on BTA21 | http://www.marc.usda.gov/ |
| DIK2492 | 18.3 |
| AGLA233 | 21.2 |
| ILSTS095 | 23.7 |
| DIK4602 | 24.3 |
| BM103 | 29.8 |
| DIK4001 | 30.0 |
| IDVGA-45 | 30.9 |
| DIK2481 | 33.7 |
| INRA103 | 35.9 |
| BMS2815 | 41.7 |
| DIK2842 | 41.7 |
| DIK3036 | 47.8 |
| DIK4391 | 52.1 |
| DIK2913 | 57.1 |

In a preferred embodiment of the invention, the at least one genetic marker is located in the region from about 18.3 cM to about 30.0 cM (http://www.marc.usda.gov/) on the bovine chromosome BTA21. In one embodiment the at least one genetic marker is located on the bovine chromosome BTA21 in the region flanked by and including the markers DIK2492 and DIK4001. The at least one genetic marker is selected from the group of markers shown in Table 15d:

TABLE 15d

| | Relative position (cM) |
|---|---|
| Marker on BTA21 | http://www.marc.usda.gov/ |
| DIK2492 | 18.3 |
| AGLA233 | 21.2 |
| ILSTS095 | 23.7 |
| DIK4602 | 24.3 |
| BM103 | 29.8 |
| DIK4001 | 30.0 |

In a preferred embodiment of the invention, the at least one genetic marker is located in the region from about 30.9 cM to about 47.8 cM (http://www.marc.usda.gov/) on the bovine chromosome BTA21. In one embodiment the at least one genetic marker is located on the bovine chromosome BTA21 in the region flanked by and including the markers IDVGA-45 and DIK3036. The at least one genetic marker is selected from the group of markers shown in Table 15e:

TABLE 15e

| | Relative position (cM) |
|---|---|
| Marker on BTA21 | http://www.marc.usda.gov/ |
| IDVGA-45 | 30.9 |
| DIK2481 | 33.7 |
| INRA103 | 35.9 |
| BMS2815 | 41.7 |
| DIK2842 | 41.7 |
| DIK3036 | 47.8 |

In a preferred embodiment of the invention, the at least one genetic marker is located in the region from about 33.7 cM to about 41.7 cM (http://www.marc.usda.gov/) on the bovine chromosome BTA21. In one embodiment the at least one genetic marker is located on the bovine chromosome BTA21 in the region flanked by and including the markers DIK2481 and BMS2815. The at least one genetic marker is selected from the group of markers shown in Table 15f:

TABLE 15f

| | Relative position (cM) |
|---|---|
| Marker on BTA21 | http://www.marc.usda.gov/ |
| DIK2481 | 33.7 |
| INRA103 | 35.9 |
| BMS2815 | 41.7 |

In a preferred embodiment of the invention, the at least one genetic marker is located in the region from about 5.5 cM to about 61.2 cM (http://www.marc.usda.gov/) on the bovine chromosome BTA21. In one embodiment the at least one genetic marker is located on the bovine chromosome BTA21 in the region flanked by and including the markers DIK5182 and BM846. The at least one genetic marker is selected from the group of markers shown in Table 15g:

TABLE 15g

| | Relative position (cM) |
|---|---|
| Marker on BTA21 | http://www.marc.usda.gov/ |
| DIK5182 | 5.5 |
| DIK3036 | 47.8 |
| DIK4391 | 52.1 |
| DIK2913 | 57.1 |
| BM846 | 61.2 |

### BTA22

In one embodiment of the invention the at least one genetic marker is located on the bovine chromosome BTA22. In one specific embodiment of the present invention, the at least one genetic marker is located in the region from about 0.0 cM to about 82.9 cM (http://www.marc.usda.gov/) on the bovine chromosome BTA22. In one embodiment the at least one genetic marker is located on the bovine chromosome BTA22 in the region flanked by and including the markers CSSM26 and BM4102. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 16a:

TABLE 16a

| | Relative position (cM) |
|---|---|
| Marker on BTA22 | http://www.marc.usda.gov/ |
| CSSM26 | 0.0 |
| INRA026 | 2.9 |
| BM1558 | 19.1 |
| BM3628 | 47.1 |
| BMS875 | 64.1 |
| BM4102 | 82.9 |

In a preferred embodiment of the invention, the at least one genetic marker is located in the region from about 2.9 cM to about 82.9 cM (http://www.marc.usda.gov/) on the bovine chromosome BTA22. In one embodiment the at least one genetic marker is located on the bovine chromosome BTA22 in the region flanked by and including the markers INRA026 and BM4102. The at least one genetic marker is selected from the group of markers shown in Table 16b:

TABLE 16b

| | Relative position (cM) |
|---|---|
| Marker on BTA22 | http://www.marc.usda.gov/ |
| INRA026 | 2.9 |
| BM1558 | 19.1 |
| BM3628 | 47.1 |
| BMS875 | 64.1 |
| BM4102 | 82.9 |

In a preferred embodiment of the invention, the at least one genetic marker is located in the region from about 2.9 cM to about 47.1 cM (http://www.marc.usda.gov/) on the bovine chromosome BTA22. In one embodiment the at least one genetic marker is located on the bovine chromosome BTA22 in the region flanked by and including the markers INRA026 and BM3628. The at least one genetic marker is selected from the group of markers shown in Table 16c:

TABLE 16c

| | Relative position (cM) |
|---|---|
| Marker on BTA22 | http://www.marc.usda.gov/ |
| INRA026 | 2.9 |
| BM1558 | 19.1 |
| BM3628 | 47.1 |

In a preferred embodiment of the invention, the at least one genetic marker is located in the region from about 19.1 cM to about 47.1 cM (http://www.marc.usda.gov/) on the bovine chromosome BTA22. In one embodiment the at least one genetic marker is located on the bovine chromosome BTA22 in the region flanked by and including the markers BM1558 and BM3628. The at least one genetic marker is selected from the group of markers shown in Table 16d:

TABLE 16d

| | Relative position (cM) |
|---|---|
| Marker on BTA22 | http://www.marc.usda.gov/ |
| BM1558 | 19.1 |
| BM3628 | 47.1 |

In a preferred embodiment of the invention, the at least one genetic marker is located in the region from about 19.1 cM to about 82.9 cM (http://www.marc.usda.gov/) on the bovine chromosome BTA22. In one embodiment the at least one genetic marker is located on the bovine chromosome BTA22 in the region flanked by and including the markers BM1558 and BM4102. The at least one genetic marker is selected from the group of markers shown in Table 16e:

TABLE 16e

| | Relative position (cM) |
|---|---|
| Marker on BTA22 | http://www.marc.usda.gov/ |
| BM1558 | 19.1 |
| BM3628 | 47.1 |
| BMS875 | 64.1 |
| BM4102 | 82.9 |

In a preferred embodiment of the invention, the at least one genetic marker is located in the region from about 47.1 cM to about 82.9 cM (http://www.marc.usda.gov/) on the bovine chromosome BTA22. In one embodiment the at least one genetic marker is located on the bovine chromosome BTA22 in the region flanked by and including the markers BM3628 and BM4102. The at least one genetic marker is selected from the group of markers shown in Table 16f:

TABLE 16f

| | Relative position (cM) |
|---|---|
| Marker on BTA22 | http://www.marc.usda.gov/ |
| BM3628 | 47.1 |
| BMS875 | 64.1 |
| BM4102 | 82.9 |

In a preferred embodiment of the invention, the at least one genetic marker is located in the region from about 64.1 cM to about 82.9 cM (http://www.marc.usda.gov/) on the bovine chromosome BTA22. In one embodiment the at least one genetic marker is located on the bovine chromosome BTA22 in the region flanked by and including the markers BMS875 and BM4102. The at least one genetic marker is selected from the group of markers shown in Table 16g:

TABLE 16g

| | Relative position (cM) |
|---|---|
| Marker on BTA22 | http://www.marc.usda.gov/ |
| BMS875 | 64.1 |
| BM4102 | 82.9 |

### BTA24

In one embodiment of the invention the at least one genetic marker is located on the bovine chromosome BTA24. In one specific embodiment of the present invention, the at least one genetic marker is located in the region from about 6.2 cM to about 65.9 cM (http://www.marc.usda.gov/) on the bovine chromosome BTA24. In one embodiment the at least one genetic marker is located on the bovine chromosome BTA24 in the region flanked by and including the markers BMS917 and BMS3024. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 17a:

TABLE 17a

| | Relative position (cM) |
|---|---|
| Marker on BTA24 | http://www.marc.usda.gov/ |
| BMS917 | 6.2 |
| BM7151 | 8.2 |
| BM226 | 8.2 |
| BMS2526 | 8.2 |
| TGLA351 | 11.1 |
| BM7228 | 19.3 |
| CSSM23 | 20.6 |
| BMS2270 | 23.7 |
| ILSTS065 | 27.4 |
| BMS1862 | 35.5 |
| BMS466 | 48.8 |
| INRA090 | 56.3 |
| BMS1926 | 61.2 |
| BMS3024 | 65.9 |

In a preferred embodiment of the invention, the at least one genetic marker is located in the region from about 8.2 cM to about 65.9 cM (http://www.marc.usda.gov/) on the bovine chromosome BTA24. In one embodiment the at least one genetic marker is located on the bovine chromosome BTA24 in the region flanked by and including the markers BM7151 and BMS3024. The at least one genetic marker is selected from the group of markers shown in Table 17b:

TABLE 17b

| | Relative position (cM) |
|---|---|
| Marker on BTA24 | http://www.marc.usda.gov/ |
| BM7151 | 8.2 |
| BM226 | 8.2 |
| BMS2526 | 8.2 |
| TGLA351 | 11.1 |
| BM7228 | 19.3 |
| CSSM23 | 20.6 |
| BMS2270 | 23.7 |
| ILSTS065 | 27.4 |
| BMS1862 | 35.5 |
| BMS466 | 48.8 |
| INRA090 | 56.3 |
| BMS1926 | 61.2 |
| BMS3024 | 65.9 |

In a preferred embodiment of the invention, the at least one genetic marker is located in the region from about 8.2 cM to about 35.5 cM (http://www.marc.usda.gov/) on the bovine chromosome BTA24. In one embodiment the at least one genetic marker is located on the bovine chromosome BTA24 in the region flanked by and including the markers BM7151 and BMS1862. The at least one genetic marker is selected from the group of markers shown in Table 17c:

TABLE 17c

| | Relative position (cM) |
|---|---|
| Marker on BTA24 | http://www.marc.usda.gov/ |
| BM7151 | 8.2 |
| BM226 | 8.2 |
| BMS2526 | 8.2 |
| TGLA351 | 11.1 |
| BM7228 | 19.3 |
| CSSM23 | 20.6 |
| BMS2270 | 23.7 |
| ILSTS065 | 27.4 |
| BMS1862 | 35.5 |

In a preferred embodiment of the invention, the at least one genetic marker is located in the region from about 11.1 cM to about 23.7 cM (http://www.marc.usda.gov/) on the bovine chromosome BTA24. In one embodiment the at least one genetic marker is located on the bovine chromosome BTA24 in the region flanked by and including the markers TGLA351 and BMS2270. The at least one genetic marker is selected from the group of markers shown in Table 17d:

TABLE 17d

| | Relative position (cM) |
|---|---|
| Marker on BTA24 | http://www.marc.usda.gov/ |
| TGLA351 | 11.1 |
| BM7228 | 19.3 |
| CSSM23 | 20.6 |
| BMS2270 | 23.7 |

In a preferred embodiment of the invention, the at least one genetic marker is located in the region from about 35.5 cM to about 65.9 cM (http://www.marc.usda.gov/) on the bovine chromosome BTA24. In one embodiment the at least one genetic marker is located on the bovine chromosome BTA24 in the region flanked by and including the markers BMS1862 and BMS3024. The at least one genetic marker is selected from the group of markers shown in Table 17e:

TABLE 17e

| | Relative position (cM) |
|---|---|
| Marker on BTA24 | http://www.marc.usda.gov/ |
| BMS1862 | 35.5 |
| BMS466 | 48.8 |
| INRA090 | 56.3 |
| BMS1926 | 61.2 |
| BMS3024 | 65.9 |

In a preferred embodiment of the invention, the at least one genetic marker is located in the region from about 48.8 cM to about 61.2 cM (http://www.marc.usda.gov/) on the bovine chromosome BTA24. In one embodiment the at least one genetic marker is located on the bovine chromosome BTA24 in the region flanked by and including the markers BMS466 and BMS1926. The at least one genetic marker is selected from the group of markers shown in Table 17f:

TABLE 17f

| | Relative position (cM) |
|---|---|
| Marker on BTA24 | http://www.marc.usda.gov/ |
| BMS466 | 48.8 |
| INRA090 | 56.3 |
| BMS1926 | 61.2 |

In a preferred embodiment of the invention, the at least one genetic marker is located in the region from about 48.8 cM to about 56.3 cM (http://www.marc.usda.gov/) on the bovine chromosome BTA24. In one embodiment the at least one genetic marker is located on the bovine chromosome BTA24 in the region flanked by and including the markers BMS466 and INRA090. The at least one genetic marker is selected from the group of markers shown in Table 17g:

TABLE 17g

| | Relative position (cM) |
|---|---|
| Marker on BTA24 | http://www.marc.usda.gov/ |
| BMS466 | 48.8 |
| INRA090 | 56.3 |

In a preferred embodiment of the invention, the at least one genetic marker is located in the region from about 56.3 cM to about 61.2 cM (http://www.marc.usda.gov/) on the bovine chromosome BTA24. In one embodiment the at least one genetic marker is located on the bovine chromosome BTA24 in the region flanked by and including the markers INRA090 and BMS1926. The at least one genetic marker is selected from the group of markers shown in Table 17h:

TABLE 17h

| | Relative position (cM) |
|---|---|
| Marker on BTA24 | http://www.marc.usda.gov/ |
| INRA090 | 56.3 |
| BMS1926 | 61.2 |

### BTA25

In one embodiment of the invention the at least one genetic marker is located on the bovine chromosome BTA25. In one specific embodiment of the present invention, the at least one genetic marker is located in the region from about 7.2 cM to about 61.7 cM (http://www.marc.usda.gov/) on the bovine chromosome BTA25. In one embodiment the at least one genetic marker is located on the bovine chromosome BTA25 in the region flanked by and including the markers ILSTS102 and AF5. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 18a:

TABLE 18a

| | Relative position (cM) |
|---|---|
| Marker on BTA25 | http://www.marc.usda.gov/ |
| ILSTS102 | 7.2 |
| BMS2843 | 22.6 |
| BM737 | 31.6 |
| ILSTS046 | 33.3 |
| BMS1353 | 46.4 |
| AF5 | 61.7 |

In a preferred embodiment of the invention, the at least one genetic marker is located in the region from about 7.2 cM to about 31.6 cM (http://www.marc.usda.gov/) on the bovine chromosome BTA25. In one embodiment the at least one genetic marker is located on the bovine chromosome BTA25 in the region flanked by and including the markers ILSTS102 and BM737. The at least one genetic marker is selected from the group of markers shown in Table 18b:

TABLE 18b

| | Relative position (cM) |
|---|---|
| Marker on BTA25 | http://www.marc.usda.gov/ |
| ILSTS102 | 7.2 |
| BMS2843 | 22.6 |
| BM737 | 31.6 |

In a preferred embodiment of the invention, the at least one genetic marker is located in the region from about 7.2 cM to about 22.6 cM (http://www.marc.usda.gov/) on the bovine chromosome BTA25. In one embodiment the at least one genetic marker is located on the bovine chromosome BTA25 in the region flanked by and including the markers ILSTS102 and BMS2843. The at least one genetic marker is selected from the group of markers shown in Table 18c:

TABLE 18c

| | Relative position (cM) |
|---|---|
| Marker on BTA25 | http://www.marc.usda.gov/ |
| ILSTS102 | 7.2 |
| BMS2843 | 22.6 |

In a preferred embodiment of the invention, the at least one genetic marker is located in the region from about 31.6 cM to about 61.7 cM (http://www.marc.usda.gov/) on the bovine chromosome BTA25. In one embodiment the at least one genetic marker is located on the bovine chromosome BTA25 in the region flanked by and including the markers BM737 and AF5. The at least one genetic marker is selected from the group of markers shown in Table 18d:

TABLE 18d

| | Relative position (CM) |
|---|---|
| Marker on BTA25 | http://www.marc.usda.gov/ |
| BM737 | 31.6 |
| ILSTS046 | 33.3 |
| BMS1353 | 46.4 |
| AF5 | 61.7 |

In a preferred embodiment of the invention, the at least one genetic marker is located in the region from about 33.3 cM to about 46.4 cM (http://www.marc.usda.gov/) on the bovine chromosome BTA25. In one embodiment the at least one genetic marker is located on the bovine chromosome BTA25 in the region flanked by and including the markers ILSTS046 and BMS1353. The at least one genetic marker is selected from the group of markers shown in Table 18e:

TABLE 18e

| | Relative position (cM) |
|---|---|
| Marker on BTA25 | http://www.marc.usda.gov/ |
| ILSTS046 | 33.3 |
| BMS1353 | 46.4 |

In a preferred embodiment of the invention, the at least one genetic marker is located in the region from about 46.4 cM to about 61.7 cM (http://www.marc.usda.gov/) on the bovine chromosome BTA25. In one embodiment the at least one genetic marker is located on the bovine chromosome BTA25 in the region flanked by and including the markers BMS1353 and AF5. The at least one genetic marker is selected from the group of markers shown in Table 18f:

TABLE 18f

| | Relative position (cM) |
|---|---|
| Marker on BTA25 | http://www.marc.usda.gov/ |
| BMS1353 | 46.4 |
| AF5 | 61.7 |

### BTA26

In one embodiment of the invention the at least one genetic marker is located on the bovine chromosome BTA26. In one specific embodiment of the present invention, the at least one genetic marker is located in the region from about 2.8 cM to about 66.8 cM (http://www.marc.usda.gov/) on the bovine chromosome BTA26. In one embodiment the at least one genetic marker is located on the bovine chromosome BTA26 in the region flanked by and including the markers BMS651 and BM7237. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 19a:

TABLE 19a

| | Relative position (cM) |
|---|---|
| Marker on BTA26 | http://www.marc.usda.gov/ |
| BMS651 | 2.8 |
| HEL11 | 22.9 |
| BMS332 | 31.7 |
| RM026 | 37.6 |
| BM9284 | 41.6 |
| RME40 | 43.2 |
| IDVGA-59 | 53.1 |
| BMS882 | 53.5 |
| BM804 | 60.5 |
| BM7237 | 66.8 |

In a preferred embodiment of the invention, the at least one genetic marker is located in the region from about 2.8 cM to about 60.5 cM (http://www.marc.usda.gov/) on the bovine chromosome BTA26. In one embodiment the at least one genetic marker is located on the bovine chromosome BTA26 in the region flanked by and including the markers BMS651 and BM804. The at least one genetic marker is selected from the group of markers shown in Table 19b:

TABLE 19b

| | Relative position (cM) |
|---|---|
| Marker on BTA26 | http://www.marc.usda.gov/ |
| BMS651 | 2.8 |
| HEL11 | 22.9 |
| BMS332 | 31.7 |
| RM026 | 37.6 |
| BM9284 | 41.6 |
| RME40 | 43.2 |
| IDVGA-59 | 53.1 |
| BMS882 | 53.5 |
| BM804 | 60.5 |

In a preferred embodiment of the invention, the at least one genetic marker is located in the region from about 2.8 cM to about 37.6 cM (http://www.marc.usda.gov/) on the bovine chromosome BTA26. In one embodiment the at least one genetic marker is located on the bovine chromosome BTA26 in the region flanked by and including the markers BMS651 and RM026. The at least one genetic marker is selected from the group of markers shown in Table 19c:

TABLE 19c

| | Relative position (cM) |
|---|---|
| Marker on BTA26 | http://www.marc.usda.gov/ |
| BMS651 | 2.8 |
| HEL11 | 22.9 |
| BMS332 | 31.7 |
| RM026 | 37.6 |

In a preferred embodiment of the invention, the at least one genetic marker is located in the region from about 22.9 cM to about 31.7 cM (http://www.marc.usda.gov/) on the bovine chromosome BTA26. In one embodiment the at least one genetic marker is located on the bovine chromosome BTA26 in the region flanked by and including the markers HEL11 and BMS332. The at least one genetic marker is selected from the group of markers shown in Table 19d:

TABLE 19d

| | Relative position (cM) |
|---|---|
| Marker on BTA26 | http://www.marc.usda.gov/ |
| HEL11 | 22.9 |
| BMS332 | 31.7 |

In a preferred embodiment of the invention, the at least one genetic marker is located in the region from about 31.7 cM to about 41.6 cM (http://www.marc.usda.gov/) on the bovine chromosome BTA26. In one embodiment the at least one genetic marker is located on the bovine chromosome BTA26 in the region flanked by and including the markers BMS332 and BM9284. The at least one genetic marker is selected from the group of markers shown in Table 19e:

TABLE 19e

| | Relative position (cM) |
|---|---|
| Marker on BTA26 | http://www.marc.usda.gov/ |
| BMS332 | 31.7 |
| RM026 | 37.6 |
| BM9284 | 41.6 |

In a preferred embodiment of the invention, the at least one genetic marker is located in the region from about 37.6 cM to about 66.8 cM (http://www.marc.usda.gov/) on the bovine chromosome BTA26. In one embodiment the at least one genetic marker is located on the bovine chromosome BTA26 in the region flanked by and including the markers RM026 and BM7237. The at least one genetic marker is selected from the group of markers shown in Table 19f:

TABLE 19f

| | Relative position (cM) |
|---|---|
| Marker on BTA26 | http://www.marc.usda.gov/ |
| RM026 | 37.6 |
| BM9284 | 41.6 |
| RME40 | 43.2 |
| IDVGA-59 | 53.1 |
| BMS882 | 53.5 |
| BM804 | 60.5 |
| BM7237 | 66.8 |

In a preferred embodiment of the invention, the at least one genetic marker is located in the region from about 37.6 cM to about 43.2 cM (http://www.marc.usda.gov/) on the bovine chromosome BTA26. In one embodiment the at least one genetic marker is located on the bovine chromosome BTA26 in the region flanked by and including the markers RM026 and RME40. The at least one genetic marker is selected from the group of markers shown in Table 19g:

TABLE 19g

| | Relative position (cM) |
|---|---|
| Marker on BTA26 | http://www.marc.usda.gov/ |
| RM026 | 37.6 |
| BM9284 | 41.6 |
| RME40 | 43.2 |

In a preferred embodiment of the invention, the at least one genetic marker is located in the region from about 43.2 cM to about 66.8 cM (http://www.marc.usda.gov/) on the bovine chromosome BTA26. In one embodiment the at least one genetic marker is located on the bovine chromosome BTA26 in the region flanked by and including the markers RME40 and BM7237. The at least one genetic marker is selected from the group of markers shown in Table 19h:

TABLE 19h

| | Relative position (cM) |
|---|---|
| Marker on BTA26 | http://www.marc.usda.gov/ |
| RME40 | 43.2 |
| IDVGA-59 | 53.1 |
| BMS882 | 53.5 |
| BM804 | 60.5 |
| BM7237 | 66.8 |

In a preferred embodiment of the invention, the at least one genetic marker is located in the region from about 53.1 cM to about 60.5 cM (http://www.marc.usda.gov/) on the bovine chromosome BTA26. In one embodiment the at least one genetic marker is located on the bovine chromosome BTA26 in the region flanked by and including the markers IDVGA-59 and BM804. The at least one genetic marker is selected from the group of markers shown in Table 19i:

TABLE 19i

| | Relative position (cM) |
|---|---|
| Marker on BTA26 | http://www.marc.usda.gov/ |
| IDVGA-59 | 53.1 |
| BMS882 | 53.5 |
| BM804 | 60.5 |

### BTA28

In one embodiment of the invention the at least one genetic marker is located on the bovine chromosome BTA28. In one specific embodiment of the present invention, the at least one genetic marker is located in the region from about 8.0 cM to about 59.6 cM (http://www.marc.usda.gov/) on the bovine chromosome BTA28. In one embodiment the at least one genetic marker is located on the bovine chromosome BTA28 in the region flanked by and including the markers BMC6020 and BMC2208. The at least one genetic marker is significant for the calving traits SB, CD and/or CS. In a particular embodiment the at least one genetic marker is significant for example the trait SB, such as CD, for example CS. However, in a further embodiment the at least one genetic marker is significant for the traits in any combination. The at least one genetic marker is selected from the group of markers shown in Table 20a:

TABLE 20a

| | Relative position (cM) |
|---|---|
| Marker on BTA28 | http://www.marc.usda.gov/ |
| BMC6020 | 8.0 |
| ETH1112 | 16.9 |
| BL25 | 24.8 |
| DIK2955 | 38.0 |
| BMS2608 | 38.5 |
| BMS2658 | 43.0 |
| DIK713 | 45.9 |
| BMS1714 | 49.4 |
| DIK5056 | 50.5 |
| DIK5323 | 55.9 |
| DIK4862 | 59.6 |
| BMC2208 | 59.6 |

In a preferred embodiment of the invention, the at least one genetic marker is located in the region from about 8.0 cM to about 24.8 cM (http://www.marc.usda.gov/) on the bovine chromosome BTA28. In one embodiment the at least one genetic marker is located on the bovine chromosome BTA28 in the region flanked by and including the markers BMC6020 and BL25. The at least one genetic marker is selected from the group of markers shown in Table 20b:

TABLE 20b

| | Relative position (cM) |
|---|---|
| Marker on BTA28 | http://www.marc.usda.gov/ |
| BMC6020 | 8.0 |
| ETH1112 | 16.9 |
| BL25 | 24.8 |

In a preferred embodiment of the invention, the at least one genetic marker is located in the region from about 16.9 cM to about 24.8 cM (http://www.marc.usda.gov/) on the bovine chromosome BTA28. In one embodiment the at least one genetic marker is located on the bovine chromosome BTA28 in the region flanked by and including the markers ETH1112 and BL25. The at least one genetic marker is selected from the group of markers shown in Table 20c:

TABLE 20c

| | Relative position (cM) |
|---|---|
| Marker on BTA28 | http://www.marc.usda.gov/ |
| ETH1112 | 16.9 |
| BL25 | 24.8 |

In a preferred embodiment of the invention, the at least one genetic marker is located in the region from about 24.8 cM to about 50.5 cM (http://www.marc.usda.gov/) on the bovine chromosome BTA28. In one embodiment the at least one genetic marker is located on the bovine chromosome BTA28 in the region flanked by and including the markers BL25 and DIK5056. The at least one genetic marker is selected from the group of markers shown in Table 20d:

TABLE 20d

| | Relative position (cM) |
|---|---|
| Marker on BTA28 | http://www.marc.usda.gov/ |
| BL25 | 24.8 |
| DIK2955 | 38.0 |
| BMS2608 | 38.5 |
| BMS2658 | 43.0 |
| DIK713 | 45.9 |
| BMS1714 | 49.4 |
| DIK5056 | 50.5 |

In a preferred embodiment of the invention, the at least one genetic marker is located in the region from about 38.0 cM to about 45.9 cM (http://www.marc.usda.gov/) on the bovine chromosome BTA28. In one embodiment the at least one genetic marker is located on the bovine chromosome BTA28 in the region flanked by and including the markers DIK2955 and DIK713. The at least one genetic marker is selected from the group of markers shown in Table 20e:

TABLE 20e

| | Relative position (cM) |
|---|---|
| Marker on BTA28 | http://www.marc.usda.gov/ |
| DIK2955 | 38.0 |
| BMS2608 | 38.5 |
| BMS2658 | 43.0 |
| DIK713 | 45.9 |

In a preferred embodiment of the invention, the at least one genetic marker is located in the region from about 38.0 cM to about 43.0 cM (http://www.marc.usda.gov/) on the bovine chromosome BTA28. In one embodiment the at least one genetic marker is located on the bovine chromosome BTA28 in the region flanked by and including the markers DIK2955 and BMS2658. The at least one genetic marker is selected from the group of markers shown in Table 20f:

TABLE 20f

| | Relative position (cM) |
|---|---|
| Marker on BTA28 | http://www.marc.usda.gov/ |
| DIK2955 | 38.0 |
| BMS2608 | 38.5 |
| BMS2658 | 43.0 |

In a preferred embodiment of the invention, the at least one genetic marker is located in the region from about 43.0 cM to about 59.6 cM (http://www.marc.usda.gov/) on the bovine chromosome BTA28. In one embodiment the at least one genetic marker is located on the bovine chromosome BTA28 in the region flanked by and including the markers BMS2658 and BMC2208. The at least one genetic marker is selected from the group of markers shown in Table 20g:

TABLE 20g

| | Relative position (cM) |
|---|---|
| Marker on BTA28 | http://www.marc.usda.gov/ |
| BMS2658 | 43.0 |
| DIK713 | 45.9 |
| BMS1714 | 49.4 |
| DIK5056 | 50.5 |
| DIK5323 | 55.9 |
| DIK4862 | 59.6 |
| BMC2208 | 59.6 |

In a preferred embodiment of the invention, the at least one genetic marker is located in the region from about 45.9 cM to about 55.9 cM (http://www.marc.usda.gov/) on the bovine chromosome BTA28. In one embodiment the at least one genetic marker is located on the bovine chromosome BTA28 in the region flanked by and including the markers DIK713 and DIK5323. The at least one genetic marker is selected from the group of markers shown in Table 20h:

TABLE 20h

| | Relative position (cM) |
|---|---|
| Marker on BTA28 | http://www.marc.usda.gov/ |
| DIK713 | 45.9 |
| BMS1714 | 49.4 |
| DIK5056 | 50.5 |
| DIK5323 | 55.9 |

In a preferred embodiment of the invention, the at least one genetic marker is located in the region from about 49.4 cM to about 50.5 cM (http://www.marc.usda.gov/) on the bovine chromosome BTA28. In one embodiment the at least one genetic marker is located on the bovine chromosome BTA28 in the region flanked by and including the markers BMS1714 and DIK5056. The at least one genetic marker is selected from the group of markers shown in Table 20i:

TABLE 20i

| | Relative position (cM) |
|---|---|
| Marker on BTA28 | http://www.marc.usda.gov/ |
| BMS1714 | 49.4 |
| DIK5056 | 50.5 |

In a preferred embodiment of the invention, the at least one genetic marker is located in the region from about 55.9 cM to about 59.6 cM (http://www.marc.usda.gov/) on the bovine chromosome BTA28. In one embodiment the at least one genetic marker is located on the bovine chromosome BTA28 in the region flanked by and including the markers DIK5323 and BMC2208. The at least one genetic marker is selected from the group of markers shown in Table 20j:

TABLE 20j

| | Relative position (cM) |
|---|---|
| Marker on BTA28 | http://www.marc.usda.gov/ |
| DIK5323 | 55.9 |
| DIK4862 | 59.6 |
| BMC2208 | 59.6 |

In another embodiment of the present invention, the at least one genetic marker is a combination of markers, as indicated in tables 20k1 to 20k19. It is understood that the term BTA3, BTA4. BTA5, BTA7, BTA8, BTA9, BTA10, BTA11, BTA12, BTA15, BTA18, BTA19, BTA20, BTA21, BTA22, BTA24, BTA25, BTA26, and BTA28 in tables 20k1 to 20k19 is meant to comprise any regions and genetic markers located on the bovine chromosomes, respectively, as described elsewhere herein.

The tables 20k1 to 20k19 show different embodiments, wherein the combination of markers is a multiplicity of bovine chromosomes, wherein the specific chromosome in each embodiment is indicated with X.

TABLE 20k1

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | X | X | | | | | | | | | | | | | | | | | |
| 2 | X | | X | | | | | | | | | | | | | | | | |
| 3 | X | | | X | | | | | | | | | | | | | | | |
| 4 | X | | | | X | | | | | | | | | | | | | | |
| 5 | X | | | | | X | | | | | | | | | | | | | |
| 6 | X | | | | | | X | | | | | | | | | | | | |
| 7 | X | | | | | | | X | | | | | | | | | | | |
| 8 | X | | | | | | | | X | | | | | | | | | | |
| 9 | X | | | | | | | | | X | | | | | | | | | |
| 10 | X | | | | | | | | | | X | | | | | | | | |
| 11 | X | | | | | | | | | | | X | | | | | | | |
| 12 | X | | | | | | | | | | | | X | | | | | | |
| 13 | X | | | | | | | | | | | | | X | | | | | |
| 14 | X | | | | | | | | | | | | | | X | | | | |
| 15 | X | | | | | | | | | | | | | | | X | | | |
| 16 | X | | | | | | | | | | | | | | | | X | | |
| 17 | X | | | | | | | | | | | | | | | | | X | |
| 18 | X | | | | | | | | | | | | | | | | | | X |
| 19 | X | | X | | X | | | | | | X | | | | | | | X | |
| 20 | X | | X | | X | | | | | | X | | | | | | | | |
| 21 | X | | X | | X | | | | | | | | | | | | | | |
| 22 | X | | X | | | | | | | | X | | | | | | | | |
| 23 | X | | | | X | | | | | | X | | | | | | | X | |
| 24 | X | | | | | | | | | | X | | | | | | | X | |

TABLE 20k2

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | X | X | | | | | | | | | | | | | | | | |
| 2 | | X | | X | | | | | | | | | | | | | | | |
| 3 | | X | | | X | | | | | | | | | | | | | | |
| 4 | | X | | | | X | | | | | | | | | | | | | |
| 5 | | X | | | | | X | | | | | | | | | | | | |
| 6 | | X | | | | | | X | | | | | | | | | | | |
| 7 | | X | | | | | | | X | | | | | | | | | | |
| 8 | | X | | | | | | | | X | | | | | | | | | |
| 9 | | X | | | | | | | | | X | | | | | | | | |
| 10 | | X | | | | | | | | | | X | | | | | | | |
| 11 | | X | | | | | | | | | | | X | | | | | | |
| 12 | | X | | | | | | | | | | | | X | | | | | |
| 13 | | X | | | | | | | | | | | | | X | | | | |
| 14 | | X | | | | | | | | | | | | | | X | | | |
| 15 | | X | | | | | | | | | | | | | | | X | | |
| 16 | | X | | | | | | | | | | | | | | | | X | |
| 17 | | X | | | | | | | | | | | | | | | | | X |
| 18 | | X | | | X | | | X | | | | | X | | | | X | | X |
| 19 | | X | X | | X | | | | | | X | | | | | | | X | |
| 20 | | X | X | | X | | | | | | X | | | | | | | | |
| 21 | | X | X | | X | | | | | | | | | | | | | | |
| 22 | | X | X | | | | | | | | X | | | | | | | | |
| 23 | | X | | | X | | | | | | X | | | | | | | X | |
| 24 | | X | | | | | | | | | X | | | | | | | X | |

TABLE 20k3

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | X | X | | | | | | | | | | | | | | | |
| 2 | | | X | | X | | | | | | | | | | | | | | |
| 3 | | | X | | | X | | | | | | | | | | | | | |
| 4 | | | X | | | | X | | | | | | | | | | | | |
| 5 | | | X | | | | | X | | | | | | | | | | | |
| 6 | | | X | | | | | | X | | | | | | | | | | |
| 7 | | | X | | | | | | | X | | | | | | | | | |
| 8 | | | X | | | | | | | | X | | | | | | | | |
| 9 | | | X | | | | | | | | | X | | | | | | | |
| 10 | | | X | | | | | | | | | | X | | | | | | |
| 11 | | | X | | | | | | | | | | | X | | | | | |
| 12 | | | X | | | | | | | | | | | | X | | | | |
| 13 | | | X | | | | | | | | | | | | | X | | | |
| 14 | | | X | | | | | | | | | | | | | | X | | |
| 15 | | | X | | | | | | | | | | | | | | | X | |
| 16 | | | X | | | | | | | | | | | | | | | | X |
| 17 | | | X | | | X | | X | | | X | | X | | X | | X | X | |
| 18 | | | X | | | | | | | | | | | | | | | | |
| 19 | | | X | | X | | | | | | X | | | | | | | X | |
| 20 | | | X | | X | | | | | | X | | | | | | | | |
| 21 | | | X | | X | | | | | | | | | | | | | | |
| 22 | | | X | | | | | | | | X | | | | | | | | |
| 23 | | | X | | X | | | | | | X | | | | | | | X | |
| 24 | | | X | | | | | | | | X | | | | | | | X | |

TABLE 20k4

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | X | X | | | | | | | | | | | | | | |
| 2 | | | | X | | X | | | | | | | | | | | | | |
| 3 | | | | X | | | X | | | | | | | | | | | | |
| 4 | | | | X | | | | X | | | | | | | | | | | |
| 5 | | | | X | | | | | X | | | | | | | | | | |
| 6 | | | | X | | | | | | X | | | | | | | | | |
| 7 | | | | X | | | | | | | X | | | | | | | | |
| 8 | | | | X | | | | | | | | X | | | | | | | |
| 9 | | | | X | | | | | | | | | X | | | | | | |
| 10 | | | | X | | | | | | | | | | X | | | | | |
| 11 | | | | X | | | | | | | | | | | X | | | | |
| 12 | | | | X | | | | | | | | | | | | X | | | |
| 13 | | | | X | | | | | | | | | | | | | X | | |
| 14 | | | | X | | | | | | | | | | | | | | X | |
| 15 | | X | | X | X | | X | | X | | X | | | | X | | X | X | X |
| 16 | | | X | X | | X | X | X | | X | | X | | X | | X | X | | |
| 17 | | X | | X | | X | | X | | | | X | | X | X | | | | |
| 18 | | | | X | | X | | X | | X | | X | | | | X | | X | X |
| 19 | | | X | X | X | | | | | | X | | | | | | | X | |
| 20 | | | X | X | X | | | | | | X | | | | | | | | |
| 21 | | | X | X | X | | | | | | | | | | | | | | |
| 22 | | | X | X | | | | | | | X | | | | | | | | |
| 23 | | | | X | X | | | | | | X | | | | | | | X | |
| 24 | | | | X | | | | | | | X | | | | | | | X | |

TABLE 20k5

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | X | X | | | | | | | | | | | | | |
| 2 | | | | | X | | X | | | | | | | | | | | | |
| 3 | | | | | X | | | X | | | | | | | | | | | |
| 4 | | | | | X | | | | X | | | | | | | | | | |
| 5 | | | | | X | | | | | X | | | | | | | | | |
| 6 | | | | | X | | | | | | X | | | | | | | | |
| 7 | | | | | X | | | | | | | X | | | | | | | |
| 8 | | | | | X | | | | | | | | X | | | | | | |
| 9 | | | | | X | | | | | | | | | X | | | | | |
| 10 | | | | | X | | | | | | | | | | X | | | | |
| 11 | | | | | X | | | | | | | | | | | X | | | |
| 12 | | | | | X | | | | | | | | | | | | X | | |
| 13 | | | | | X | | | | | | | | | | | | | X | |
| 14 | | | | | X | | | | | | | | | | | | | | X |
| 15 | | X | | X | X | | X | | X | | X | | | | X | | X | X | X |
| 16 | | | X | X | X | X | X | X | | X | | X | | X | | X | X | | |
| 17 | | X | | X | X | X | | X | | | | X | | X | X | | | | |
| 18 | | | | X | X | X | | X | | X | | X | | | | X | | X | X |
| 19 | | | X | | X | | | | | | X | | | | | | | X | |
| 20 | | | X | | X | | | | | | X | | | | | | | | |
| 21 | | | X | | X | | | | | | | | | | | | | | |
| 22 | | | X | | X | | | | | | X | | | | | | | | |
| 23 | | | | | X | | | | | | X | | | | | | | X | |
| 24 | | | | | X | | | | | | X | | | | | | | X | |

TABLE 20k6

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | X | X | | | | | | | | | | | | |
| 2 | | | | | | X | | X | | | | | | | | | | | |
| 3 | | | | | | X | | | X | | | | | | | | | | |
| 4 | | | | | | X | | | | X | | | | | | | | | |
| 5 | | | | | | X | | | | | X | | | | | | | | |
| 6 | | | | | | X | | | | | | X | | | | | | | |
| 7 | | | | | | X | | | | | | | X | | | | | | |
| 8 | | | | | | X | | | | | | | | X | | | | | |
| 9 | | | | | | X | | | | | | | | | X | | | | |
| 10 | | | | | | X | | | | | | | | | | X | | | |
| 11 | | | | | | X | | | | | | | | | | | X | | |
| 12 | | | | | | X | | | | | | | | | | | | X | |
| 13 | | | | | | X | | | | | | | | | | | | | X |
| 14 | | X | | X | X | X | X | | X | | X | | | | X | | X | X | X |
| 15 | | | X | X | X | X | X | X | | X | | X | | X | | X | X | | |
| 16 | | X | | X | X | X | | X | | | | X | | X | X | | | | |
| 17 | | | | X | X | X | | X | | X | | X | | | | X | | X | X |
| 18 | | X | | | | X | | | X | | X | | | X | | | | | |
| 19 | | | X | | X | X | | | | | X | | | | | | | X | |
| 20 | | | X | | X | X | | | | | X | | | | | | | | |
| 21 | | | X | | X | X | | | | | | | | | | | | | |
| 22 | | | X | | | X | | | | | X | | | | | | | | |
| 23 | | | | | X | X | | | | | X | | | | | | | X | |
| 24 | | | | | | X | | | | | X | | | | | | | X | |

TABLE 20k7

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | X | X | | | | | | | | | | | |
| 2 | | | | | | | X | | X | | | | | | | | | | |
| 3 | | | | | | | X | | | X | | | | | | | | | |
| 4 | | | | | | | X | | | | X | | | | | | | | |
| 5 | | | | | | | X | | | | | X | | | | | | | |
| 6 | | | | | | | X | | | | | | X | | | | | | |
| 7 | | | | | | | X | | | | | | | X | | | | | |
| 8 | | | | | | | X | | | | | | | | X | | | | |
| 9 | | | | | | | X | | | | | | | | | X | | | |
| 10 | | | | | | | X | | | | | | | | | | X | | |
| 11 | | | | | | | X | | | | | | | | | | | X | |
| 12 | | | | | | | X | | | | | | | | | | | | X |
| 13 | | X | | X | X | X | X | | X | | X | | | | X | | X | X | X |
| 14 | | | X | X | | X | X | X | | X | | X | | X | | X | X | | |
| 15 | | X | | | | X | X | X | | | | X | | X | X | | | | |
| 16 | | | | X | X | X | X | X | | X | | X | | | | X | | X | X |
| 17 | | X | | | | X | X | | X | | X | | | X | | | | | |
| 18 | | | | | | | X | | | | | | | | | | | | |
| 19 | | | X | | X | | X | | | | X | | | | | | | X | |
| 20 | | | X | | X | | X | | | | X | | | | | | | | |
| 21 | | | X | | X | | X | | | | | | | | | | | | |
| 22 | | | X | | | | X | | | | X | | | | | | | | |
| 23 | | | | | X | | X | | | | X | | | | | | | X | |
| 24 | | | | | | | X | | | | X | | | | | | | X | |

TABLE 20k8

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | X | X | | | | | | | | | | |
| 2 | | | | | | | | X | | X | | | | | | | | | |
| 3 | | | | | | | | X | | | X | | | | | | | | |
| 4 | | | | | | | | X | | | | X | | | | | | | |
| 5 | | | | | | | | X | | | | | X | | | | | | |
| 6 | | | | | | | | X | | | | | | X | | | | | |
| 7 | | | | | | | | X | | | | | | | X | | | | |
| 8 | | | | | | | | X | | | | | | | | X | | | |
| 9 | | | | | | | | X | | | | | | | | | X | | |
| 10 | | | | | | | | X | | | | | | | | | | X | |
| 11 | | | | | | | | X | | | | | | | | | | | X |
| 12 | | X | | X | X | X | X | X | X | | X | | | | X | | X | X | X |
| 13 | | | X | X | | X | X | X | | X | | X | | X | | X | X | | |
| 14 | | X | | | | X | X | X | | | | X | | X | X | | | | |
| 15 | | | | X | X | X | X | X | | X | | X | | | | X | | X | X |
| 16 | | X | | | | X | X | X | X | | X | | | X | | | | | |
| 17 | | | X | | X | | | X | | | | | X | | | | X | | |
| 18 | | | | X | X | | X | X | | X | | | X | | X | | X | | |
| 19 | | | X | | X | | | X | | | X | | | | | | | X | |
| 20 | | | X | | X | | | X | | | X | | | | | | | | |
| 21 | | | X | | X | | | X | | | | | | | | | | | |
| 22 | | | X | | | | | X | | | X | | | | | | | | |
| 23 | | | | | X | | | X | | | X | | | | | | | X | |
| 24 | | | | | | | | X | | | X | | | | | | | X | |

TABLE 20k9

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | X | X | | | | | | | | | |
| 2 | | | | | | | | | X | | X | | | | | | | | |
| 3 | | | | | | | | | X | | | X | | | | | | | |
| 4 | | | | | | | | | X | | | | X | | | | | | |
| 5 | | | | | | | | | X | | | | | X | | | | | |
| 6 | | | | | | | | | X | | | | | | X | | | | |
| 7 | | | | | | | | | X | | | | | | | X | | | |
| 8 | | | | | | | | | X | | | | | | | | X | | |
| 9 | | | | | | | | | X | | | | | | | | | X | |
| 10 | | | | | | | | | X | | | | | | | | | | X |
| 11 | | | | | X | | X | | X | X | | X | | | X | | X | | X |
| 12 | | X | X | | | X | | X | X | X | X | | | X | | X | | X | |
| 13 | X | | X | | X | | | | X | | X | | | | | | | X | |
| 14 | X | | X | | X | | | | X | | X | | | | | | | | |
| 15 | X | | X | | X | | | | X | | | | | | | | | | |
| 16 | X | | X | | | | | | X | | X | | | | | | | | |
| 17 | | | X | | X | | | | X | | X | | | | | | | X | |
| 18 | | | X | | | | | | X | | | | | | | | | | |
| 19 | | | X | | X | | | | X | | X | | | | | | | | |
| 20 | | | X | | X | | | | X | | | | | | | | | X | |
| 21 | | | X | | | | | | X | | X | | | | | | | X | |
| 22 | | | X | | | | | | X | | | | | | | | | X | |
| 23 | | | | | X | | | | X | | X | | | | | | | | |
| 24 | X | | | | | | | | X | | X | | | | | | | X | |

TABLE 20k10

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | X | X | | | | | | | | |
| 2 | | | | | | | | | | X | | X | | | | | | | |
| 3 | | | | | | | | | | X | | | X | | | | | | |
| 4 | | | | | | | | | | X | | | | X | | | | | |
| 5 | | | | | | | | | | X | | | | | X | | | | |
| 6 | | | | | | | | | | X | | | | | | X | | | |
| 7 | | | | | | | | | | X | | | | | | | X | | |
| 8 | | | | | | | | | | X | | | | | | | | X | |
| 9 | | | | | | | | | | X | | | | | | | | | X |
| 10 | | | | | X | | X | | | X | | X | | | X | | X | | X |
| 11 | | X | X | | | X | | X | | X | X | | | X | | X | | X | |
| 12 | X | | X | | X | | | | | X | X | | | | | | | X | |
| 13 | X | | X | | X | | | | | X | X | | | | | | | | |
| 14 | X | | X | | X | | | | | X | | | | | | | | | |
| 15 | X | | X | | | | | | | X | X | | | | | | | | |
| 16 | | | X | | X | | | | | X | X | | | | | | | X | |
| 17 | | | X | | | | | | | X | | | | | | | | | |
| 18 | | | X | | X | | | | | X | X | | | | | | | | |
| 19 | | | X | | X | | | | | X | | | | | | | | X | |
| 20 | | | X | | | | | | | X | X | | | | | | | X | |
| 21 | | | X | | | | | | | X | | | | | | | | X | |
| 22 | | | | | X | | | | | X | X | | | | | | | | |
| 23 | X | | | | | | | | | X | X | | | | | | | X | |
| 24 | X | | | | | | | | | X | X | | | | | | | X | |

TABLE 20k11

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | | X | X | | | | | | | |
| 2 | | | | | | | | | | | X | | X | | | | | | |
| 3 | | | | | | | | | | | X | | | X | | | | | |
| 4 | | | | | | | | | | | X | | | | X | | | | |
| 5 | | | | | | | | | | | X | | | | | X | | | |
| 6 | | | | | | | | | | | X | | | | | | X | | |
| 7 | | | | | | | | | | | X | | | | | | | X | |
| 8 | | | | | | | | | | | X | | | | | | | | X |
| 9 | | X | | | X | | | X | | X | X | | X | | X | X | | X | |
| 10 | X | | | X | | | X | | X | X | X | X | | X | X | | X | | |
| 11 | | | X | | | | X | | X | X | X | | X | | | | | | X |
| 12 | | X | X | | | X | | X | | X | X | | | X | | X | | X | |
| 13 | X | | X | | X | | | | | | X | | | | | | | X | |
| 14 | X | | X | | X | | | | | | X | | | | | | | | |
| 15 | X | | X | | X | | | | | | X | | | | | | | | |
| 16 | X | | X | | | | | | | | X | | | | | | | | |
| 17 | | | X | | X | | | | | | X | | | | | | | X | |
| 18 | | | X | | | | | | | | X | | | | | | | | |
| 19 | | | X | | X | | | | | | X | | | | | | | | |
| 20 | | | X | | X | | | | | | X | | | | | | | X | |
| 21 | | | X | | | | | | | | X | | | | | | | X | |
| 22 | | | X | | | | | | | | X | | | | | | | X | |
| 23 | | | | | X | | | | | | X | | | | | | | | |
| 24 | X | | | | | | | | | | X | | | | | | | X | |

TABLE 20k12

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | | | X | X | | | | | | |
| 2 | | | | | | | | | | | | X | | X | | | | | |
| 3 | | | | | | | | | | | | X | | | X | | | | |
| 4 | | | | | | | | | | | | X | | | | X | | | |
| 5 | | | | | | | | | | | | X | | | | | X | | |
| 6 | | | | | | | | | | | | X | | | | | | X | |
| 7 | | | | | | | | | | | | X | | | | | | | X |
| 8 | | | X | X | X | X | X | X | | X | | X | | X | | X | X | | |
| 9 | | X | | X | X | X | | X | | | | X | | X | X | | | | |
| 10 | | | | X | X | X | | X | | X | | X | | | | X | | X | X |
| 11 | | X | | | | X | | | X | | X | | | X | | | | | |
| 12 | | | X | | X | X | | | | | X | | | | | | | X | |
| 13 | X | | X | | X | | | | | | X | X | | | | | | X | |
| 14 | X | | X | | X | | | | | | X | X | | | | | | | |
| 15 | X | | X | | X | | | | | | | X | | | | | | | |
| 16 | X | | X | | | | | | | | X | X | | | | | | | |
| 17 | | | X | | X | | | | | | X | X | | | | | | X | |
| 18 | | | X | | | | | | | | | X | | | | | | | |
| 19 | | | X | | X | | | | | | X | X | | | | | | | |
| 20 | | | X | | X | | | | | | | X | | | | | | X | |
| 21 | | | X | | | | | | | | X | X | | | | | | X | |
| 22 | | | X | | | | | | | | | X | | | | | | X | |
| 23 | | | | | X | | | | | | X | X | | | | | | | |
| 24 | X | | | | | | | | | | X | X | | | | | | X | |

TABLE 20k13

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | | | | X | X | | | | | |
| 2 | | | | | | | | | | | | | X | | X | | | | |
| 3 | | | | | | | | | | | | | X | | | X | | | |
| 4 | | | | | | | | | | | | | X | | | | X | | |
| 5 | | | | | | | | | | | | | X | | | | | X | |
| 6 | | | | | | | | | | | | | X | | | | | | X |
| 7 | | X | | | X | | | X | | X | | | X | | X | X | | X | |
| 8 | X | | | X | | | X | | X | X | | X | X | X | X | | X | | |
| 9 | | | X | | | | X | | X | X | X | | X | | | | | | X |
| 10 | | X | X | | | X | | X | | X | | | X | X | | X | | X | |
| 11 | X | | X | | X | | | | | | X | | X | | | | | X | |
| 12 | X | | X | | X | | | | | | X | | X | | | | | | |
| 13 | X | | X | | X | | | | | | X | | X | | | | | | |
| 14 | X | | X | | | | | | | | X | | X | | | | | | |
| 15 | | | X | | X | | | | | | X | | X | | | | | X | |
| 16 | | | X | | | | | | | | X | | X | | | | | | |
| 17 | | | X | | X | | | | | | | | X | | | | | | |
| 18 | | | X | | X | | | | | | | | X | | | | | X | |
| 19 | | | X | | | | | | | | | | X | | | | | X | |
| 20 | | | X | | | | | | | | X | | X | | | | | X | |
| 21 | | | | | X | | | | | | | | X | | | | | | |
| 22 | X | | | | | | | | | | X | | X | | | | | X | |
| 23 | | | | | X | | | | | | | | X | | | | | X | |
| 24 | | | | | | | | | | | | | X | | | | | X | |

TABLE 20k14

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | | | | | X | X | | | | |
| 2 | | | | | | | | | | | | | | X | | X | | | |
| 3 | | | | | | | | | | | | | | X | | | X | | |
| 4 | | | | | | | | | | | | | | X | | | | X | |
| 5 | | | | | | | | | | | | | | X | | | | | X |
| 6 | | X | | | X | | | X | | X | | | X | | X | X | | X | |
| 7 | X | | | X | | | X | | X | X | | X | X | X | X | | X | | |
| 8 | | | X | | | | X | | X | X | X | | X | | | | | | X |
| 9 | | | | | X | | X | | | | | X | | X | X | | X | | X |
| 10 | | X | X | | | X | | X | | | X | | | X | | X | | X | |
| 11 | X | | X | | X | | | | | | X | | | X | | | | X | |
| 12 | X | | X | | X | | | | | | X | | | X | | | | | |
| 13 | X | | X | | X | | | | | | | | | X | | | | | |
| 14 | X | | X | | | | | | | | X | | | X | | | | | |
| 15 | | | X | | X | | | | | | X | | | X | | | | X | |
| 16 | | | X | | | | | | | | | | | X | | | | | |
| 17 | | | X | | X | | | | | | X | | | X | | | | | |
| 18 | | | X | | X | | | | | | | | | X | | | | X | |
| 19 | | | X | | | | | | | | X | | | X | | | | X | |
| 20 | | | X | | | | | | | | | | | X | | | | X | |
| 21 | | | | | X | | | | | | X | | | X | | | | | |
| 22 | X | | | | | | | | | | X | | | X | | | | X | |
| 23 | X | | | | | | | | | | X | | | X | | | | X | |
| 24 | | | | | | | | | | | X | | | X | | | | X | |

TABLE 20k15

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | | | | | | X | X | | | |
| 2 | | | | | | | | | | | | | | | X | | X | | |
| 3 | | | | | | | | | | | | | | | X | | | X | |
| 4 | | | | | | | | | | | | | | | X | | | | X |
| 5 | | X | | | X | | | X | | X | | | X | | X | X | | X | |
| 6 | X | | | X | | | X | | X | X | | X | X | X | X | | X | | |
| 7 | | | X | | | | X | | X | X | X | | X | | X | | | | X |
| 8 | | | | | X | | X | | | | | X | | | X | | X | | X |
| 9 | | X | X | | | X | | X | | | X | | | | X | X | | X | |
| 10 | X | | X | | X | | | | | | X | | | | X | | | X | |
| 11 | X | | X | | X | | | | | | X | | | | X | | | | |
| 12 | X | | X | | X | | | | | | | | | | X | | | | |
| 13 | X | | X | | | | | | | | X | | | | X | | | | |
| 14 | | | X | | X | | | | | | X | | | | X | | | X | |
| 15 | | | X | | | | | | | | | | | | X | | | | |
| 16 | | | X | | X | | | | | | X | | | | X | | | | |
| 17 | | | X | | X | | | | | | | | | | X | | | X | |
| 18 | | | X | | | | | | | | X | | | | X | | | X | |
| 19 | | | X | | | | | | | | | | | | X | | | X | |
| 20 | | | | | X | | | | | | X | | | | X | | | | |
| 21 | X | | | | | | | | | | X | | | | X | | | X | |
| 22 | X | | | | | | | | | | X | | | | X | | | X | |
| 23 | | | | | | | | | | | X | | | | X | | | X | |
| 24 | | | | | | | | | | | X | | | | X | | | X | |

TABLE 20k16

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | | | | | | | X | X | | |
| 2 | | | | | | | | | | | | | | | | X | | X | |
| 3 | | | | | | | | | | | | | | | | X | | | X |
| 4 | | X | | | X | | | X | | X | | | X | | | X | | X | |
| 5 | X | | | X | | | X | | X | X | | X | X | X | | X | X | | |
| 6 | | | X | | | | X | | X | X | X | | X | | | X | | | X |
| 7 | | | | | X | | X | | | | | X | | | | X | X | | X |
| 8 | | X | X | | | X | | X | | | X | | | | | X | | X | |
| 9 | X | | X | | X | | | | | | X | | | | | X | | X | |
| 10 | X | | X | | X | | | | | | X | | | | | X | | | |
| 11 | X | | X | | X | | | | | | | | | | | X | | | |
| 12 | X | | X | | | | | | | | X | | | | | X | | | |
| 13 | | | X | | X | | | | | | X | | | | | X | | X | |
| 14 | | | X | | | | | | | | | | | | | X | | | |
| 15 | | | X | | X | | | | | | X | | | | | X | | | |
| 16 | | | X | | X | | | | | | | | | | | X | | X | |
| 17 | | | X | | | | | | | | X | | | | | X | | X | |
| 18 | | | X | | | | | | | | | | | | | X | | X | |
| 19 | | | | | X | | | | | | X | | | | | X | | | |
| 20 | X | | | | | | | | | | X | | | | | X | | X | |
| 21 | X | | | | | | | | | | X | | | | | X | | X | |
| 22 | | | | | | | | | | | X | | | | | X | | X | |
| 23 | | | | | | | | | | | X | | | | | X | | X | |
| 24 | | | | | | | | | | | X | | | | | X | | X | |

TABLE 20k17

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | | | | | | | | X | X | |
| 2 | | | | | | | | | | | | | | | | | X | | X |
| 3 | | X | | | X | | | X | | X | | | X | | | | X | X | |
| 4 | X | | | X | | | X | | X | X | | X | X | X | | | X | | |
| 5 | | | X | | | | X | | X | X | X | | X | | | | X | | X |
| 6 | | | | | X | | X | | | | | X | | | | | X | | X |
| 7 | | X | X | | | X | | X | | | X | | | | | | X | X | |
| 8 | X | | X | | X | | | | | | X | | | | | | X | X | |
| 9 | X | | X | | X | | | | | | X | | | | | | X | | |
| 10 | X | | X | | X | | | | | | | | | | | | X | | |
| 11 | X | | X | | | | | | | | X | | | | | | X | | |
| 12 | | | X | | X | | | | | | X | | | X | | | X | X | |
| 13 | | | X | | | | | | | | | | | | X | | X | | |
| 14 | | | X | | X | | | | | | X | | | X | | | X | | |
| 15 | | | X | | X | | | | | | | | | | X | X | X | X | |
| 16 | | | X | | | | | | | | X | | | | | | X | X | |
| 17 | | | X | | | | | | | | | | | X | | X | X | X | |
| 18 | | | | | X | | | | | | X | | | | | | X | | |
| 19 | X | | | | | | | | | | X | | | | | X | X | X | |
| 20 | X | | | | | | | | | | X | | | | | | X | X | |
| 21 | | X | | X | X | | | | | | X | | | | | | X | X | |
| 22 | | | | | | | X | | | | X | | | X | | | X | X | |
| 23 | | X | X | | X | X | | | | | X | | | | | | X | X | |
| 24 | X | X | X | X | X | X | X | X | X | X | X | X | X | X | X | X | X | X | X |

TABLE 20k18

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | | | | | | | | | | | | | | | | | X | X |
| 2 | X | | X | | X | | | | | | X | | | | | | | X | |
| 3 | X | | X | | X | | | | | | X | | | | | | | X | |
| 4 | X | | X | | X | | | | | | | | | | | | | X | |
| 5 | X | | X | | | | | | | | X | | | | | | | X | |
| 6 | | | X | | X | | | | | | X | | | | | | | X | |
| 7 | | | X | | X | | | | | | | | | | | | | X | |
| 8 | | | X | | | | | | | | X | | | | | | | X | |
| 9 | | | X | | X | | | | | | | | | | | | | X | |
| 10 | | | X | | | | | | | | X | | | | | | | X | |
| 11 | | | X | | | | | | | | | | | | | | | X | |
| 12 | | | | | X | | | | | | X | | | | | | | X | |
| 13 | X | | | | | | | | | | X | | | | | | | X | |
| 14 | X | | | | X | | | | | | X | | | | | | | X | |
| 15 | X | | X | | X | | | X | | X | X | | X | | X | | X | X | |
| 16 | X | | | | | | | | | | | | | | | | | X | |
| 17 | | | | | | X | | | X | | X | | | X | | X | | X | |
| 18 | | X | | X | | | X | | | X | | X | | | X | X | X | X | |
| 19 | | | X | | X | | | | | | X | | | | | | | X | |
| 20 | | | X | | X | | | | | | X | | | | | | | X | |
| 21 | | | X | | X | | | | | | | | | | | | | X | |
| 22 | | | X | | | | | | | | X | | | | | | | X | |
| 23 | | X | | | X | | X | X | | | X | | | | | | | X | |
| 24 | | | | | | | X | | | | X | | X | | X | | | X | |

TABLE 20k19

| | BTA | | | | | | | | | | | | | | | | | | |
|---|---|---|---|---|---|---|---|---|---|---|---|---|---|---|---|---|---|---|---|
| Embodiment | 3 | 4 | 5 | 7 | 8 | 9 | 10 | 11 | 12 | 15 | 18 | 19 | 20 | 21 | 22 | 24 | 25 | 26 | 28 |
| 1 | | X | | | X | | | X | | X | | | X | | | | | X | X |
| 2 | X | | | X | | | X | | X | X | | X | X | X | | | | | X |
| 3 | | | X | | | | X | | X | X | X | | X | | | | | | X |
| 4 | | | | | X | | X | | | | | X | | | | X | | | X |
| 5 | | X | X | | | X | | X | | | X | | | | | | | X | X |
| 6 | X | | X | | X | | | | | | X | | | | | | | X | X |
| 7 | X | | X | | X | | | | | | X | | | | | | | | X |
| 8 | X | | X | | X | | X | | X | | | X | | X | | X | X | | X |
| 9 | X | | X | | | | | | | | X | | | | | | | | X |
| 10 | | | X | | X | | | | | | X | | | X | | | | X | X |
| 11 | | | X | | | | | | | | | | | | X | | | | X |
| 12 | | | X | | X | | | | | | X | | | X | | | | | X |
| 13 | | | X | | X | | | | | | | | | | X | X | | X | X |
| 14 | | | X | | | | | | | | X | | | | | | | X | X |
| 15 | | | X | | | | | | | | | | | X | | X | | X | X |
| 16 | | | | | X | | | | | | X | | | | | | | | X |
| 17 | X | | | | | | | | | | X | | | | | X | | X | X |
| 18 | X | | | | | | | | | | X | | | | | | | X | X |
| 19 | | X | | X | X | | | | | | X | | | | | | | X | X |
| 20 | | | | | | | X | | | | X | | | X | | | | X | X |
| 21 | | X | X | | X | X | | | | | X | | | | | | | X | X |
| 22 | X | | X | | | | | | | | X | | | | | | | | X |
| 23 | X | | | | X | | | | | | X | | | | | | | X | |
| 24 | X | | | | | | | | | | X | | | | | | | X | |

### Detection

The detection of the presence or absence of a genetic marker allele according to the present invention may be conducted on the DNA sequence of the bovine chromosomes BTA3, BTA4, BTA5, BTA7, BTA8, BTA9, BTA10, BTA11, BTA12, BTA15, BTA18, BTA19, BTA20, BTA21, BTA22, BTA24, BTA25, BTA26, and/or BTA28 specified elsewhere herein according to the present invention or a complementary sequence as well as on transcriptional (mRNA) and translational products (polypeptides, proteins) therefrom.

It will be apparent to the person skilled in the art that there are a large number of analytical procedures which may be used to detect the presence or absence of variant nucleotides at one or more of positions mentioned herein in the specified region. Mutations or polymorphisms within or flanking the specified region can be detected by utilizing a number of techniques. Nucleic acid from any nucleated cell can be used as the starting point for such assay techniques, and may be isolated according to standard nucleic acid preparation procedures that are well known to those of skill in the art. In general, the detection of allelic variation requires a mutation discrimination technique, optionally an amplification reaction and a signal generation system.

A number of mutation detection techniques are listed in Table 21. Some of the methods listed in Table 21 are based on the polymerase chain reaction (PCR), wherein the method according to the present invention includes a step for amplification of the nucleotide sequence of interest in the presence of primers based on the nucleotide sequence of the variable nucleotide sequence. The methods may be used in combination with a number of signal generation systems, a selection of which is also listed in Table 22.

TABLE 21

| | |
|---|---|
| General | DNA sequencing, Sequencing by hybridisation, |
| techniques | SNAPshot |
| Scanning | Single-strand conformation polymorphism analysis, |
| techniques | Denaturing gradient gel electrophoresis, Temperature |
| | gradient gel electrophoresis, Chemical mismatch |
| | cleavage, cleavage, heteroduplex analysis, enzymatic |
| | mismatch cleavage |
| Hybridisation | Solid phase hybridisation: Dot blots, Multiple allele |
| based | specific diagnostic assay (MASDA), Reverse dot blots, |
| techniques | Oligonucleotide arrays (DNA Chips) |
| | Solution phase hybridisation: Taqman-U.S. Pat. No. |
| | 5,210,015 & 5,487,972 (Hoffmann-La Roche), Molecular |
| | Beacons -- Tyagi et al (1996), Nature Biotechnology, 14, |
| | 303; WO 95/13399 (Public Health Inst., New York), |
| | Lightcycler, optionally in combination with Fluorescence |
| | resonance energy transfer (FRET). |
| Extension | Amplification refractory mutation system (ARMS), |
| based | Amplification refractory mutation system linear extension |
| techniques | (ALEX) - European Patent No. EP 332435 B1 (Zeneca |
| | Limited), Competitive oligonucleotide priming system |
| | (COPS) - Gibbs et al (1989), Nucleic Acids Research, |
| | 17, 2347. |
| Incorporation | Mini-sequencing, Arrayed primer extension (APEX) |
| based | |
| techniques | |
| Restriction | Restriction fragment length polymorphism (RFLP), |
| Enzyme | Restriction site generating PCR |
| based | |
| techniques | |
| Ligation based | Oligonucleotide ligation assay (OLA) |
| techniques | |
| Other | Invader assay |
| Various Signal | Fluorescence: |
| Generation or | Fluorescence resonance energy transfer (FRET), |
| Detection | Fluorescence quenching, Fluorescence polarisation-- |
| Systems | United Kingdom Patent No. 2228998 (Zeneca Limited) |
| Other | Chemiluminescence, Electrochemiluminescence, Raman, |
| | Radioactivity, Colorimetric, Hybridisation protection |
| | assay, Mass spectrometry |

Further amplification techniques are listed in Table 2. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997.

The detection of genetic markers can according to one embodiment of the present invention be achieved by a number of techniques known to the skilled person, including typing of microsatellites or short tandem repeats (STR), restriction fragment length polymorphisms (RFLP), detection of deletions or insertions, random amplified polymorphic DNA (RAPIDs) or the typing of single nucleotide polymorphisms by methods such as restriction fragment length polymerase chain reaction, allele-specific oligomer hybridisation, oligomer-specific ligation assays, hybridisation with PNA or locked nucleic acids (LNA) probes.

TABLE 22

| | |
|---|---|
| Further amplification techniques | Self sustained replication (SSR), |
| | Nucleic acid sequence based |
| | amplification (NASBA), |
| | Ligase chain reaction (LCR), |
| | Strand displacement amplification |
| | (SDA) |

A primer of the present invention is a nucleic acid molecule sufficiently complementary to the sequence on which it is based and of sufficiently length to selectively hybridise to the corresponding region of a nucleic acid molecule intended to be amplified. The primer is able to prime the synthesis of the corresponding region of the intended nucleic acid molecule in the methods described above. Similarly, a probe of the present invention is a molecule for example a nucleic acid molecule of sufficient length and sufficiently complementary to the nucleic acid sequence of interest which selectively binds to the nucleic acid sequence of interest under high or low stringency conditions.

### Sample

The method according to the present invention includes analyzing a sample of a bovine subject, wherein said sample may be any suitable sample capable of providing the bovine genetic material for use in the method. The bovine genetic material may for example be extracted, isolated and purified if necessary from a blood sample, a tissue samples (for example spleen, buccal smears), clipping of a body surface (hairs or nails), milk and/or semen. The samples may be fresh or frozen.

The sequence polymorphisms of the invention comprise at least one nucleotide difference, such as at least two nucleotide differences, for example at least three nucleotide differences, such as at least four nucleotide differences, for example at least five nucleotide differences, such as at least six nucleotide differences, for example at least seven nucleotide differences, such as at least eight nucleotide differences, for example at least nine nucleotide differences, such as 10 nucleotide differences. The nucleotide differences comprise nucleotide differences, deletion and/or insertion or any combination thereof.

### Primers

The primers that may be used according to the present invention are shown in Table 22. The in Table 22 specified primer pairs may be used individually or in combination with one or more primer pairs of Table 22.

The design of such primers or probes will be apparent to the molecular biologist of ordinary skill. Such primers are of any convenient length such as up to 50 bases, up to 40 bases, more conveniently up to 30 bases in length, such as for example 8-25 or 8-15 bases in length. In general such primers will comprise base sequences entirely complementary to the corresponding wild type or variant locus in the region. However, if required one or more mismatches may be introduced, provided that the discriminatory power of the oligonucleotide probe is not unduly affected. The primers/probes of the invention may carry one or more labels to facilitate detection.

In one embodiment, the primers and/or probes are capable of hybridizing to and/or amplifying a subsequence hybridizing to a single nucleotide polymorphism containing the sequence delineated by the markers as shown herein.

The primer nucleotide sequences of the invention further include: (a) any nucleotide sequence that hybridizes to a nucleic acid molecule of the delineated region(s) or its complementary sequence or RNA products under stringent conditions, e.g., hybridization to filter-bound DNA in 6× sodium chloride/sodium citrate (SSC) at about 45° C. followed by one or more washes in 0.2×SSC/0.1% Sodium Dodecyl Sulfate (SDS) at about 50-65° C., or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & sons, Inc., New York, at pp. 6.3.1-6.3.6 and 2.10.3). Preferably the nucleic acid molecule that hybridizes to the nucleotide sequence of (a) and (b), above, is one that comprises the complement of a nucleic acid molecule of the region s or r or a complementary sequence or RNA product thereof. In a preferred embodiment, nucleic acid molecules comprising the nucleotide sequences of (a) and (b), comprises nucleic acid molecule of RAI or a complementary sequence or RNA product thereof.

Among the nucleic acid molecules of the invention are deoxyoligonucleotides (“oligos”) which hybridize under highly stringent or stringent conditions to the nucleic acid molecules described above. In general, for probes between 14 and 70 nucleotides in length the Melting Temperature™ is calculated using the formula:

Tm(° C.)=81.5+16.6(log [monovalent cations (molar)])+0.41(% G+C)−(500/N)

where N is the length of the probe. If the hybridization is carried out in a solution containing formamide, the melting temperature is calculated using the equation Tm(° C.)=81.5+16.6(log [monovalent cations (molar)])+0.41 (% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

Exemplary highly stringent conditions may refer for example to washing in 6×SSC/0.05% sodium pyrophosphate at 37° C. (for about 14-base oligos), 48° C. (for about 17-base oligos), 55° C. (for about 20-base oligos), and 60° C. (for about 23-base oligos). Accordingly, the invention further provides nucleotide primers or probes which detect the r region polymorphisms of the invention. The assessment may be conducted by means of at least one nucleic acid primer or probe, such as a primer or probe of DNA, RNA or a nucleic acid analogue such as peptide nucleic acid (PNA) or locked nucleic acid (LNA).

According to one aspect of the present invention there is provided an allele-specific oligonucleotide probe capable of detecting a polymorphism at one or more of positions in the delineated regions 1.

The allele-specific oligonucleotide probe is preferably 5-50 nucleotides, more preferably about 5-35 nucleotides, more preferably about 5-30 nucleotides, more preferably at least 9 nucleotides.

### Determination of Linkage

In order to detect whether the genetic marker is present in the genetic material, standard methods well known to persons skilled in the art may be applied, for example by the use of nucleic acid amplification. In order to determine whether the genetic marker is genetically linked to the calving traits, a permutation test can be applied when the regression method is used (Doerge and Churchill, 1996), or the Piepho-method can be applied (Piepho, 2001) when the variance components method is used. The principle of the permutation test is well described by Doerge and Churchill (1996), whereas the Piepho-method is well described by Piepho (2001). Significant linkage in the within family analysis using the regression method, a 1000 permutations were made using the permutation test (Doerge and Churchill, 1996). A threshold at the 5% chromosome wide level was considered to be significant evidence for linkage between the genetic marker and the calving traits. In addition, the QTL was confirmed in different sire families. For the across family analysis and multi-trait analysis with the variance component method the piepho method was used to determine the significance level (Piepho, 2001). A threshold at the 5% chromosome wide level was considered to be significant evidence for linkage between the genetic marker and the calving traits.

### Kit

Another aspect of the present invention relates to a diagnostic kit for use in detecting the presence or absence in a bovine subject of at least one genetic marker associated with bovine calving characteristics, comprising at least one oligonucleotide sequence, wherein the nucleotide sequences are selected from any of SEQ ID NO.: 1 to SEQ ID NO.: 558 and/or any combination thereof.

Genotyping of a bovine subject in order to establish the genetic determinants of calving traits for that subject according to the present invention can be based on the analysis of genomic DNA which can be provided using standard DNA extraction methods as described herein. The genomic DNA may be isolated and amplified using standard techniques such as the polymerase chain reaction using oligonucleotide primers corresponding (complementary) to the polymorphic marker regions. Additional steps of purifying the DNA prior to amplification reaction may be included. Thus, a diagnostic kit for establishing calving characteristics comprises, in a separate packing, at least one oligonucleotide sequence selected from the group of sequences shown in table 23 and any combinations thereof.

### EXAMPLES

### Experimental Design

A total genome scan for QTL affecting calving traits, was carried out in the Danish Holstein population. Marker and phenotypic data were collected according to the granddaughter design (Weller et al., 1990), which included 34 sires with 2042 progeny-tested sons. Numbers of sons per sire ranged from 20 to 106. Sires and their sons were genotyped for marker information whereas phenotypic records were taken from granddaughter performances. Numbers of daughters of each son ranged between 70 and 100. The marker data set included a total of 384 microsatellites covering all 29 Bos Taurus chromosomes.

Purification of genomic DNA

Genomic DNA was purified from semen according to the following protocol:

After thawing the semen-straw, both ends of the straw were cut away with a pair of scissors and the content of semen transferred to a 1.5 ml eppendorf tube. 1 ml of 0.9% NaCl was used to flush the straw into the tube. The tube was then centrifuged for 5 minutes at 2000 rpm, followed by removal of the supernatant. This washing step was repeated twice.

Then 300 μl buffer S (10 mM Tris HCl pH 8, 100 mM NaCl, 10 mM EDTA pH 8; 0.5% SDS), 20 μl 1 M DTT and 20 μl pronase (20 mg/ml) (Boehringer) are added to the tube. After mixing the tubes are incubated over night with slow rotation where after 180 μl saturated NaCl is added followed by vigorous agitation for 15 seconds. The tube is the centrifuged for 15 minutes at 11000 rpm. 0.4 ml of the supernatant is transferred to a 2 ml tube and 1 ml of 96% ethanol is added, mixing is achieved by slow rotation of the tube. The tube is then centrifuged for 10 minutes at 11000 rpm. Remove the supernatant by pouring away the liquid, wash the pellet with 70% ethanol (0.2 ml) and centrifuge again for 10 minutes at 11000 rpm. Pour away the ethanol, dry the pellet and resuspend in 0.5 ml of TE-buffer) for 30 minutes at 55° C.

### Amplification Procedures

PCR reactions were run in a volume of 8 μl using TEMPase (GeneChoice) polymerase and reaction buffer I as provided by the supplier (GeneChoice). Usually 5 different markers are included in each multiplex PCR. 1 μl DNA, 0.1 μl TEMPase enzyme, 0.2 mM dNTPs, 1.2 mM MgCl2, 0.3 μM each primer.

The PCR mixtures were subjected to initial denaturation at 94° C. for 15 min (for TEMPase). Subsequently, the samples were cycled for 10 cycles with touchdown, i.e. the temperature is lowered 1° C. at each cycle (denaturation at 94° C. 30″, annealing at 67° C. 45″, elongation 72° C. 30″), after which the samples were cycled for 20 cycles with normal PCR conditions (denaturation at 94° C. 30″, annealing at 58° C. 45″, elongation 72° C. 30) PCR cycling was terminated by I cycle at 72° C. 30′ and the PCR machine was programmed to cooling down the samples at 4° C. for ‘ever’.

The nucleotide sequence of the primers used for detecting the markers is shown in Table 23. The sequence is listed from the 5′ end.

TABLE 23

| | Forward Primer F | |
|---|---|---|
| Marker name | Reverse Primer R | SEQ ID NO.: |
| BTA3: | | |
| TNRA006 | F AGGAATATCTGTATCAACCTCAGTC | SEQ ID NO.: 1 |
| | R CTGAGCTGGGGTGGGAGCTATAAATA | SEQ ID NO.: 2 |
| UWCA7 | F TGTAGCTCCCTGGAGGAGAA | SEQ ID NO.: 3 |
| | R GCAAATACAACCCAGTCTGGTG | SEQ ID NO.: 4 |
| ILSTS096 | F GTGACCTGGAGAAGTTTTCC | SEQ ID NO.: 5 |
| | R ACCACGCTCTGACTTGTAGC | SEQ ID NO.: 6 |
| DIK4403 | F CGTGCTGCAACTGAGAGTTC | SEQ ID NO.: 7 |
| | R GCTGTATAGCAAAGTGACCCAGT | SEQ ID NO.: 8 |
| RME23 | F AGAACAAATGTGACACTCACA | SEQ ID NO.: 9 |
| | R GTGAGTACAGGCGCTTTCTG | SEQ ID NO.: 10 |
| BMS963 | F GGAGGATGAAGGAGTCTTTGG | SEQ ID NO.: 11 |
| | R AATTTACCACAGTCCACCGC | SEQ ID NO.: 12 |
| BMS819 | F AAAGAATTGGACCTGACTGAGC | SEQ ID NO.: 13 |
| | R GCTTTCACTTCTGCTGGCTT | SEQ ID NO.: 14 |
| FCGR1 | F GGTCTTCATTGGTGTTTTCTCC | SEQ ID NO.: 15 |
| | R GAGCTGCCCTAGATGAGGTG | SEQ ID NO.: 16 |
| BL41 | F CCTCTGCCATCTTTATTCCG | SEQ ID NO.: 17 |
| | R AAGATCAACTTATTCCTCACAGTGG | SEQ ID NO.: 18 |
| DIK4353C | F TGAACTTTAGGGCAGCATGA | SEQ ID NO.: 19 |
| | R AAGACTGAGATGTGGGGAAAA | SEQ ID NO.: 20 |
| INRA003 | F CTGGAGGTGTGTGAGCCCCATTTA | SEQ ID NO.: 21 |
| | R CTAAGAGTCGAAGGTGTGACTAGG | SEQ ID NO.: 22 |
| BMS2790 | F AAGACAAGGACTTTCAGCCC | SEQ ID NO.: 23 |
| | R AAAGAGTCGGACATTACTGAGC | SEQ ID NO.: 24 |
| ILSTS029 | F TGTTTTGATGGAACACAGCC | SEQ ID NO.: 25 |
| | R TGGATTTAGACCAGGGTTGG | SEQ ID NO.: 26 |
| BM220 | F TTTTCTACTGCCCAACAAAGTG | SEQ ID NO.: 27 |
| | R TAGGTACCATAGCCTAGCCAAG | SEQ ID NO.: 28 |
| INRA123 | F TCTAGAGGATCCCCGCTGAC | SEQ ID NO.: 29 |
| | R AGAGAGCAACTCCACTGTGC | SEQ ID NO.: 30 |
| BMS862 | F TATAATGCCCTCTAGATCCACTCA | SEQ ID NO.: 31 |
| | R ATGGAAAAATAAGATGTGGTATGTG | SEQ ID NO.: 32 |
| HUJ246 | F ACTCCAGTTTTCTTTCCTGGG | SEQ ID NO.: 33 |
| | R TGCCATGTAGTAGCTGTGTGC | SEQ ID NO.: 34 |
| BMS937 | F GTAGCCATGGAGACTGGACTG | SEQ ID NO.: 35 |
| | R CATTATCCCCTGTCACACACC | SEQ ID NO.: 36 |
| DIK4664 | F AACTGGCTCCAAGGTCAATG | SEQ ID NO.: 37 |
| | R TCCCCTGTCACACACCTGTA | SEQ ID NO.: 38 |
| DIK2702 | F TGCGATATTTAATGGATGTCT | SEQ ID NO.: 39 |
| | R TTCCTTTCTCCGAACTGCTC | SEQ ID NO.: 40 |
| HUJII77 | F TCCATCAAGTATTTGAGTGCAA | SEQ ID NO.: 41 |
| | R ATAGCCCTACCCACTGTTTCTG | SEQ ID NO.: 42 |
| DIK2686 | F ATGTTTTTCAGGCCAATCCA | SEQ ID NO.: 43 |
| | R TGCCCTGATTTCTCATACCC | SEQ ID NO.: 44 |
| BM7225 | F GGTGTTATGCATTCTCTAGGTGC | SEQ ID NO.: 45 |
| | R AAGAGTTAGACATGACTGAGCACG | SEQ ID NO.: 46 |
| BTA4: | | |
| BMS1788 | F ACGTCCAGATTCAGATTTCTTG | SEQ ID NO.: 47 |
| | R GGAGAGGAATCTTGCAAAGG | SEQ ID NO.: 48 |
| BMS2646 | F CAAAGCCATAAGAAGCAATTATG | SEQ ID NO.: 49 |
| BMS2646 | R CCTTCTATAGTGTGGTGAGTACCC | SEQ ID NO.: 50 |
| TGLA116 | F GCACAGTAATAAGAGTGATGGCAGA | SEQ ID NO.: 51 |
| | R TGGAGAAGATTTGGGTGTGTACCCA | SEQ ID NO.: 52 |
| INRA072 | F CTTAACTCATTCACCTCAACTG | SEQ ID NO.: 53 |
| | R AGTGATTGAGCACATTGCGCAT | SEQ ID NO.: 54 |
| BM8233 | F GCATTGGCAAGTGGATTCTT | SEQ ID NO.: 55 |
| | R AAGGCAATTAACACATACATCACC | SEQ ID NO.: 56 |
| BMS648 | F ACTTCCCATCCATCCATCAG | SEQ ID NO.: 57 |
| | R CTTCCATTCTCAGCCATCTAGC | SEQ ID NO.: 58 |
| BR6303 | F TGAGCCATAGAATTAAGATTCAAGC | SEQ ID NO.: 59 |
| | R TTTGTTCCTCTTTATTTTCTTCTGC | SEQ ID NO.: 60 |
| MGTG4B | F GAGCAGCTTCTTTCTTTCTCATCTT | SEQ ID NO.: 61 |
| | R GCTCTTGGAAGCTTATTGTATAAAG | SEQ ID NO.: 62 |
| BTA5: | | |
| BMS1095 | F AGGGATTGGTTTATGCTCTCTC | SEQ ID NO.: 63 |
| | R GTTGCAGAGTCGGACATGAC | SEQ ID NO.: 64 |
| BM6026 | F GCAACTAAGACCCAACCAAC | SEQ ID NO.: 65 |
| | R ACTGATGTGCTCAGGTATGACG | SEQ ID NO.: 66 |
| MNB-33 | F GCTTTGGTACACCCTTTAAGC | SEQ ID NO.: 67 |
| | R GAACAAATTCACAAGGGAAAAC | SEQ ID NO.: 68 |
| BMS610 | F TTTCACTGTCATCTCCCTAGCA | SEQ ID NO.: 69 |
| | R ATGTATTCATGCACACCACACA | SEQ ID NO.: 70 |
| BP1 | F AAAATCCCTTCATAACAGTGCC | SEQ ID NO.: 71 |
| | R CATCGTGAATTCCAGGGTTC | SEQ ID NO.: 72 |
| DIK4747 | F CCAAAAATTCTGGCACCAAT | SEQ ID NO.: 73 |
| | R CCTGGGCTTGTGACTAGCAT | SEQ ID NO.: 74 |
| DIK2718 | F AGGAAGGACAAGGACATTGC | SEQ ID NO.: 75 |
| | R AGAGGGTCAAAGGCTTAATGG | SEQ ID NO.: 76 |
| AGLA293 | F GAAACTCAACCCAAGACAACTCAAG | SEQ ID NO.: 77 |
| | R ATGACTTTATTCTCCACCTAGCAGA | SEQ ID NO.: 78 |
| DIK5002 | F TGTGCTGGAGGTGATAGCTG | SEQ ID NO.: 79 |
| | R TGCAGGAATATGAGAGCTGAGA | SEQ ID NO.: 80 |
| DIK4759 | F AGTTGGACCTGCCATTGTTC | SEQ ID NO.: 81 |
| | R ACTTATGTGCGTGCGTGCT | SEQ ID NO.: 82 |
| BMC1009 | F GCACCAGCAGAGAGGACATT | SEQ ID NO.: 83 |
| | R ACCGGCTATTGTCCATCTTG | SEQ ID NO.: 84 |
| CSSM034 | F CCATAACTCTGGGACTTTTCCTCA | SEQ ID NO.: 557 |
| | R ATGTTCAGCCATCTCTCCTGGTCC | SEQ ID NO.: 558 |
| RM500 | F CAGACACGACTAAGCGACCA | SEQ ID NO.: 85 |
| | R CCTACAATAAAGCACGGGGA | SEQ ID NO.: 86 |
| BMS1617 | F GCCTGCATGTGTCTGTGG | SEQ ID NO.: 87 |
| | R TCTGTGTCGGAATACCCTCC | SEQ ID NO.: 88 |
| DIK5046 | F TGAATTGTTTCTGCTTCTTGGA | SEQ ID NO.: 89 |
| | R TGCATGACTCCCCTCTCTCT | SEQ ID NO.: 90 |
| ETH10 | F GTTCAGGACTGGCCCTGCTAACA | SEQ ID NO.: 91 |
| | R CCTCCAGCCCACTTTCTCTTCTC | SEQ ID NO.: 92 |
| CSSM022 | F TCTCTCTAATGGAGTTGGTTTTTG | SEQ ID NO.: 93 |
| | R ATATCCCACTGAGGATAAGAATTC | SEQ ID NO.: 94 |
| BMS1216 | F GAGTAGAACACAACTGAGGACACA | SEQ ID NO.: 95 |
| | R CAATGCTGTGGGTACTGAGG | SEQ ID NO.: 96 |
| DIK2943 | F GGTTTCCTCAGGACATGGTG | SEQ ID NO.: 97 |
| | R CAGTCCATGAGGTTGCAGAA | SEQ ID NO.: 98 |
| BMS1248 | F GTAATGTAGCCTTTTGTGCCG | SEQ ID NO.: 99 |
| | R TCACCAACATGAGATAGTGTGC | SEQ ID NO.: 100 |
| BM315 | F TGGTTTAGCAGAGAGCACATG | SEQ ID NO.: 101 |
| | R GCTCCTAGCCCTGCACAC | SEQ ID NO.: 102 |
| BMS1658 | F ATTGATGCTTTATGATCCTCATG | SEQ ID NO.: 103 |
| | R CCCACTAAGAGAGGAGGAGG | SEQ ID NO.: 104 |
| BM2830 | F AATGGGCGTATAAACACAGATG | SEQ ID NO.: 105 |
| | R TGAGTCCTGTCACCATCAGC | SEQ ID NO.: 106 |
| BTA7: | | |
| BM7160 | F TGGATTTTTAAACACAGAATGTGG | SEQ ID NO.: 107 |
| | R TCAGCTTCTCTTTAAATTTCTCTGG | SEQ ID NO.: 108 |
| BL1067 | F AGCCAGTTTCTTCAAATCAACC | SEQ ID NO.: 109 |
| | R ATGGTTCCGCAGAGAAACAG | SEQ ID NO.: 110 |
| BMS713 | F CCAAGGGAGGAAAAATAAGTTAA | SEQ ID NO.: 111 |
| | R ACCAGCAGTAGGTTGAGGTTAA | SEQ ID NO.: 112 |
| DIK5321 | F AACCTTCACAGGCTCCTTCC | SEQ ID NO.: 113 |
| | R CCCATCTCTTGTGCCAAATC | SEQ ID NO.: 114 |
| DIK4421 | F CATCTGAATGGCCAGAATGA | SEQ ID NO.: 115 |
| | R GTCCCCTGCATGTGTCTCTC | SEQ ID NO.: 116 |
| DIK2207 | F ACATTGGCTTACGCTCACACT | SEQ ID NO.: 117 |
| | R CCTGTCTGGGTTTGTTTGCT | SEQ ID NO.: 118 |
| DIK5412 | F ATGGACAGAACAGCCTGACA | SEQ ID NO.: 119 |
| | R TGGTGAACTCAGCCTCACTG | SEQ ID NO.: 120 |
| IL4 | F GTGCTGGACATCTGCAAGTG | SEQ ID NO.: 121 |
| | R ACATTCAGGTCTGTGATCCATG | SEQ ID NO.: 122 |
| BM6105 | F ACTAATAAGAAATTCTGCATGTGTG | SEQ ID NO.: 123 |
| | R CCACCATGACTCAGAAGTAGTTC | SEQ ID NO.: 124 |
| TGLA303 | F TAATCATAAGTCAAAGTAACAGTTT | SEQ ID NO.: 125 |
| | R GATCTGGACATACAAAAGTATTAC | SEQ ID NO.: 126 |
| DIK2819 | F TTACTTTTCGTGGGCCAGAG | SEQ ID NO.: 127 |
| | R GGAACTGTGCCACATAGCAA | SEQ ID NO.: 128 |
| DIK4606 | F TCTTGGAAAGGGGAAAAAGC | SEQ ID NO.: 129 |
| | R TGCTTCATAGCACTTATCTCTTCA | SEQ ID NO.: 130 |
| BM7247 | F AGTAAGGCCTGCAGTATTTATATCC | SEQ ID NO.: 131 |
| | R AATCTTTCCCTAGAACTTACAAAGG | SEQ ID NO.: 132 |
| UWCA20 | F CTGAAACACTCTAAAAGGGTATGC | SEQ ID NO.: 133 |
| | R ATCCCAACATCCACCCATTCC | SEQ ID NO.: 134 |
| BM6117 | F GTTCTGAGGTTTGTAAAGCCC | SEQ ID NO.: 135 |
| | R GGTGAGCTACAATCCATAGGG | SEQ ID NO.: 136 |
| BMS2840 | F AGGAACCCATAGGCAGACAC | SEQ ID NO.: 137 |
| | R GCCTGGCAAAGAGAAAATTC | SEQ ID NO.: 138 |
| DIK2915 | F TCTCACCCTCACATGGTTCA | SEQ ID NO.: 139 |
| | R GTGGAGCCAAGGTGAAAGAA | SEQ ID NO.: 140 |
| BMS2258 | F CCAGCAGAAGAGAAAGATACTGA | SEQ ID NO.: 141 |
| | R AGTGGTAGAACTTCCATCTCACA | SEQ ID NO.: 142 |
| OARAE129 | F AATCCAGTGTGTGAAAGACTAATCCAG | SEQ ID NO.: 143 |
| | R GTAGATCAAGATATAGAATATTTTTCAACACC | SEQ ID NO.: 144 |
| DIK2895 | F CTCAATGACGTTTGGCTTCA | SEQ ID NO.: 145 |
| | R GGTGCCTGACTCCAATTGAT | SEQ ID NO.: 146 |
| ILSTS006 | F TGTCTGTATTTCTGCTGTGG | SEQ ID NO.: 147 |
| | R ACACGGAAGCGATCTAAACG | SEQ ID NO.: 148 |
| BL1043 | F AGTGCCAAAAGGAAGCGC | SEQ ID NO.: 149 |
| | R GACTTGACCGTTCCACCTG | SEQ ID NO.: 150 |
| BTA8: | | |
| IDVGA-11 | F CCTCTGGGTCTATCCATGTTG | SEQ ID NO.: 151 |
| | R TGGATGAATGAAGAAGATGCC | SEQ ID NO.: 152 |
| BMS1591 | F GACAAGATAGGCTTTGCATGA | SEQ ID NO.: 153 |
| | R GATAGAAATATACCAGGAGCTCACA | SEQ ID NO.: 154 |
| BMS678 | F ACCATCTACTGTGCTATGGCTT | SEQ ID NO.: 155 |
| | R GCAGAAACACAATACTCAGTGC | SEQ ID NO.: 156 |
| INRA129 | F GGGTAGCCTGTTAAAATGCAG | SEQ ID NO.: 157 |
| | R CAGTGCTGACCTCTGAAGTAAG | SEQ ID NO.: 158 |
| BMS2072 | F TGTTCAGTGCTTGTCTTAGCTG | SEQ ID NO.: 159 |
| | R TCTTCAAAGCCATCAATCATC | SEQ ID NO.: 160 |
| BMS887 | F AAGCTAACTGATATTCTGCCACA | SEQ ID NO.: 161 |
| | R TTCCCTCTCTTCCCTCTCC | SEQ ID NO.: 162 |
| URB037 | F ACTGGAGACGACTGAAGCAACC | SEQ ID NO.: 163 |
| | R GAGTGGCTGTTGCTAAATTTGG | SEQ ID NO.: 164 |
| MCM64 | F TACAGTCCATGGGGTCACAAGAG | SEQ ID NO.: 165 |
| | R TCTGAATCTACTCCCTCCTCAGAGC | SEQ ID NO.: 166 |
| CSSM047 | F TCTCTGTCTCTATCACTATATGGC | SEQ ID NO.: 167 |
| | R CTGGGCACCTGAAACTATCATCAT | SEQ ID NO.: 168 |
| BMS836 | F GAAACTCTTTTCACTCTGCGC | SEQ ID NO.: 169 |
| | R GCTCTTAGGGATTGCTTCACC | SEQ ID NO.: 170 |
| BTA9: | | |
| BMS2151 | F CCATTAAGAGGAAATTGTGTTCA | SEQ ID NO.: 171 |
| | R ATGGAGTCACTGAAAGGTACTGA | SEQ ID NO.: 172 |
| ETH225 | F GATCACCTTGCCACTATTTCCT | SEQ ID NO.: 173 |
| | R ACATGACAGCCAGCTGCTACT | SEQ ID NO.: 174 |
| ILSTS037 | F TAGGCTATGTACTGACCATGC | SEQ ID NO.: 175 |
| | R CTGAACTGAGATGACTTTGGC | SEQ ID NO.: 176 |
| BM2504 | F CAGCTTTCCATCCCCTTTC | SEQ ID NO.: 177 |
| | R CTCCCATCCCAAACACAGAC | SEQ ID NO.: 178 |
| DIK2892 | F TTGACCCTGAAAGATGTCCA | SEQ ID NO.: 179 |
| | R CACGGTTTATCAGCTTGGGTA | SEQ ID NO.: 180 |
| DIK3003 | F ACTTTCAGTTTTGGGCTGAC | SEQ ID NO.: 181 |
| | R TGTCACTAGGTAAATTGGTG | SEQ ID NO.: 182 |
| DIK3002 | F AAATGGAGGTAATGAAATAAAATA | SEQ ID NO.: 183 |
| | R CAAACCCATGGACTGTAACCT | SEQ ID NO.: 184 |
| BMS1267 | F TTCTGAATTTGATTCCCAACA | SEQ ID NO.: 185 |
| | R ACTGTTTCCTTAAAAGCTTCCC | SEQ ID NO.: 186 |
| DIK5142 | F TGGGTAAGTGGGAAAGGATG | SEQ ID NO.: 187 |
| | R CTCAGCCAGGTTGTCCTCTC | SEQ ID NO.: 188 |
| BMS555 | F GGAAAGAGTAGGTGATTCCCTG | SEQ ID NO.: 189 |
| | R ATTTAATTGTCATCCCAGGTGA | SEQ ID NO.: 190 |
| DIK5364 | F CCTCTGAAACCCCAGACTTG | SEQ ID NO.: 191 |
| | R AAAAACCCAAAACAACACACAA | SEQ ID NO.: 192 |
| UWCA9 | F CCTTCTCTGAATTTTTGTTGAAAGC | SEQ ID NO.: 193 |
| | R GGACAGAAGTGAGTGACTGAGA | SEQ ID NO.: 194 |
| DIK4720 | F CATGATATTTACCCTGTGTGTGC | SEQ ID NO.: 195 |
| | R GAGGAGCTGGAGGGCTAAAG | SEQ ID NO.: 196 |
| BMS1290 | F TTGGCACTTACTACCTCATATGTT | SEQ ID NO.: 197 |
| | R TTTTCTGGATGTTGAGCCTATT | SEQ ID NO.: 198 |
| DIK2816 | F ACCTTGGGAATCAAGGTCAT | SEQ ID NO.: 199 |
| | R CCCAGTAGTCCAGTGGCTCA | SEQ ID NO.: 200 |
| BM6436 | F AAAGACTGCTTGCCTGAAGC | SEQ ID NO.: 201 |
| | R CAACCAGTGATGCTGTACTCTG | SEQ ID NO.: 202 |
| BMS2753 | F TCAAAAAGTTGGACATGACTGA | SEQ ID NO.: 203 |
| | R AGGTTTTCAAATGAGAGACTTTTC | SEQ ID NO.: 204 |
| BM4208 | F TCAGTACACTGGCCACCATG | SEQ ID NO.: 205 |
| | R CACTGCATGCTTTTCCAAAC | SEQ ID NO.: 206 |
| BMS2819 | F GCTCACAGGTTCTGAGGACTC | SEQ ID NO.: 207 |
| | R AACTTGAAGAAGGAATGCTGAG | SEQ ID NO.: 208 |
| BMS2295 | F GCTCTGGTGACCCAGGTG | SEQ ID NO.: 209 |
| | R CTGGCAGGAGATGAGAGGAG | SEQ ID NO.: 210 |
| BMS1967 | F GGGCAGATGTGAGTAATTTTCC | SEQ ID NO.: 211 |
| | R AACTGAGCTGTATGGTGGACG | SEQ ID NO.: 212 |
| BTA10 | | |
| DIK2658 | F GCACATTGGGATCTCTCCTG | SEQ ID NO.: 213 |
| | R AAAGTCCCATCCCACAATCA | SEQ ID NO.: 214 |
| DTK2503 | F TCCTTACAACACACCATGCAA | SEQ ID NO.: 215 |
| | R CACACCCAGGCATCCATAC | SEQ ID NO.: 216 |
| CSSM38 | F TTCATATAAGCAGTTTATAAACGC | SEQ ID NO.: 217 |
| | R ATAGGATCTGGTAACTTACAGATG | SEQ ID NO.: 218 |
| BMS528 | F CTCACTCCACTGGGCTTCTC | SEQ ID NO.: 219 |
| | R TGTGTTCTCACCTCGACCAC | SEQ ID NO.: 220 |
| BM1237 | F TCATCTTGGGCATAAGACAGG | SEQ ID NO.: 221 |
| | R ATTGTTCCCAGCATCTTAGAGG | SEQ ID NO.: 222 |
| MB077 | F CACCCGTACCCTCACTGC | SEQ ID NO.: 223 |
| | R TCACAACCCTCTTCTCACCC | SEQ ID NO.: 224 |
| DIK2000 | F TGGCTTGCAACACTGCAC | SEQ ID NO.: 225 |
| | R CCCACCTACGACTGGGACTTA | SEQ ID NO.: 226 |
| BMS2742 | F GCTTCAGTTCTGCTTTTCACC | SEQ ID NO.: 227 |
| | R CTTCAGCATCTTGATTGTTGC | SEQ ID NO.: 228 |
| BMS529 | F CTCCAGGTAAGACAGGCCAC | SEQ ID NO.: 229 |
| | R CCCGATCTGTGTGTGGGT | SEQ ID NO.: 230 |
| DIK2361 | F TGTGGGTTTGATCTCTGAGT | SEQ ID NO.: 231 |
| | R TGTGTCCTCCTTTGTGGTAGAA | SEQ ID NO.: 232 |
| BM888 | F ACTAGGAGGCCATATAGGAGGC | SEQ ID NO.: 233 |
| | R GAGCTCAAAACGAGGGACAG | SEQ ID NO.: 234 |
| TGLA433 | F ATTTCTATGAAGTAGTCTTCTGACT | SEQ ID NO.: 235 |
| | R ATTTTAAAACTAGTCACGAGTGCCT | SEQ ID NO.: 236 |
| INRA037 | F GATCCTGCTTATATTTAACCAC | SEQ ID NO.: 237 |
| | R AAAATTCCATGGAGAGAGAAAC | SEQ ID NO.: 238 |
| BMS1620 | F TATGAACTCACATGGTTACCACA | SEQ ID NO.: 239 |
| | R TTGCCCAAAAATAGACCTTAAA | SEQ ID NO.: 240 |
| ILSTS070 | F GGTATTTTGAGAATGTGGGC | SEQ ID NO.: 241 |
| | R TCTTTGACCACTACCTATCC | SEQ ID NO.: 242 |
| BMS2641 | F GTGCGGAAAGGAACAGAGTC | SEQ ID NO.: 243 |
| | R AAAGCCGGACTGGAGTGTC | SEQ ID NO.: 244 |
| BMS614 | F AATGCGTGGGACTTGTTTT | SEQ ID NO.: 245 |
| | R CAATTGCTGAAGCAGTCACA | SEQ ID NO.: 246 |
| BMS2614 | F ACTTTCTTTTCCTGTGGCTCG | SEQ ID NO.: 247 |
| | R CAGAGCTGGCACCAGAGG | SEQ ID NO.: 248 |
| BTA11: | | |
| BM716 | F AGTACTTGGCTTGCTTTGCTC | SEQ ID NO.: 249 |
| | R TTAAATTTCCATCTCACCCTGG | SEQ ID NO.: 250 |
| BMS2569 | F AGAGAGGCCAAAGCTGGG | SEQ ID NO.: 251 |
| | R TTTCCTTGGGCTTCAGGAG | SEQ ID NO.: 252 |
| BM2818 | F TTCTGTGGTTGAAGAGTGTTCC | SEQ ID NO.: 253 |
| | R CAATGGCTAAGAGGTCCAGTG | SEQ ID NO.: 254 |
| INRA177-2 | F TCCAAAAGTTTCGTGACATATTG | SEQ ID NO.: 255 |
| | R CACCAGGCTTCTCTGTTGAA | SEQ ID NO.: 256 |
| INRA177 | F TCCAAAAGTTTCGTGACATATTG | SEQ ID NO.: 257 |
| | R CACCAGGCTTCTCTGTTGAA | SEQ ID NO.: 258 |
| RM096 | F TCGCAAAAAGTTGGACAAGAC | SEQ ID NO.: 259 |
| | R TTAGCAGGGTGCCTGACACTT | SEQ ID NO.: 260 |
| INRA131 | F GGTAAAATCCTGCAAAACACAG | SEQ ID NO.: 261 |
| | R TGACTGTATAGACTGAAGCAAC | SEQ ID NO.: 262 |
| BM7169 | F TGGTATGTAGTTACAGCAGCCC | SEQ ID NO.: 263 |
| | R CCATTGAAACAGACATGAATGC | SEQ ID NO.: 264 |
| BM6445 | F GTGTCTGTCAAAAGATGAATGG | SEQ ID NO.: 265 |
| | R GACAACTGCTTCTCGTTGGG | SEQ ID NO.: 266 |
| ILSTS036 | F GAGTATTATGCTTGGGAGGC | SEQ ID NO.: 267 |
| | R AGACAGGATGGGAAGTCACC | SEQ ID NO.: 268 |
| BMS1822 | F AAAGGCTTCTATTTGTGGTGG | SEQ ID NO.: 269 |
| | R TTGATGCTTTATTGTTTTCCTCT | SEQ ID NO.: 270 |
| TGLA58 | F TTCTACTCTCCAGCCTCCTCC | SEQ ID NO.: 271 |
| | R GTTGGCTCCAAGAGCAAGTC | SEQ ID NO.: 272 |
| BMS2047 | F ACTATGGACATTTGGGGCAG | SEQ ID NO.: 273 |
| | R AGTAGGTGGAGATCAAGGATGC | SEQ ID NO.: 274 |
| HUJV174 | F CAGACCAGTTTCTCAGACAAGC | SEQ ID NO.: 275 |
| | R TCATTCCTGTGTCAATACAGCC | SEQ ID NO.: 276 |
| BMS989 | F TTTGAGAACTTTTGTTTCTGAGC | SEQ ID NO.: 277 |
| | R TTATTTTGCTTTTCTGATTTTGTG | SEQ ID NO.: 278 |
| TGLA436 | F TGTATGGCTGAATGATATTCCATTT | SEQ ID NO.: 279 |
| | R CTACTGACAGATGATTAGATAAAGA | SEQ ID NO.: 280 |
| BMS460 | F TGCCCCATAGTGTAGTGCTC | SEQ ID NO.: 281 |
| | R GCCAGCAGAGAATTGTAGCA | SEQ ID NO.: 282 |
| ILSTS045 | F TTCTGGCAAACTATTCCACC | SEQ ID NO.: 283 |
| | R CATGAAAGACACAGATGACC | SEQ ID NO.: 284 |
| DIK4819 | F ATTTTTCCCAGCGCCTCTC | SEQ ID NO.: 285 |
| | R AAACAGAAGACTCAGGAAGACGA | SEQ ID NO.: 286 |
| HEL13 | F TAAGGACTTGAGATAAGGAG | SEQ ID NO.: 287 |
| | R CCATCTACCTCCATCTTAAC | SEQ ID NO.: 288 |
| BTA12: | | |
| BMS410 | F GGCTGAAAAGCTGTGGTGTT | SEQ ID NO.: 289 |
| | R TTGCCACATTTACCTTCTTTCA | SEQ ID NO.: 290 |
| BM6108 | F TTCTAATGTAGAGCAAAGTGATTGA | SEQ ID NO.: 291 |
| | R TGTAGGAGGGACAGATTGGG | SEQ ID NO.: 292 |
| BM860 | F ACCAGATTGGTGGTAGTGGTG | SEQ ID NO.: 293 |
| | R CATGCCGTGGCTAAGACC | SEQ ID NO.: 294 |
| BMS975 | F TGGAGCTAAATCAATGCGTG | SEQ ID NO.: 295 |
| | R CCCAATGGCCAATTAAGTACC | SEQ ID NO.: 296 |
| BMS1316 | F CCTTCATGGAAGAAATTTTGTG | SEQ ID NO.: 297 |
| | R GGAGTTACAGTCCATGGGTTC | SEQ ID NO.: 298 |
| BMS2724 | F GGCTGATACACAGAGACATGC | SEQ ID NO.: 299 |
| | R CCTCTCTGCCTTCTATCAGGT | SEQ ID NO.: 300 |
| BTA15: | | |
| BR3510 | F GCTGGTGGGTTGTTTACCAC | SEQ ID NO.: 301 |
| | R ACCCCGTGGACTGTAGTCTG | SEQ ID NO.: 302 |
| BMS2533 | F TGAAGTAAGTAAGCACACAAGCA | SEQ ID NO.: 303 |
| | R TTGATCATCTTTAGGTCCATCC | SEQ ID NO.: 304 |
| INRA050 | F ACAGGCTACAGTCCATGGGGTT | SEQ ID NO.: 305 |
| | R TATAGAACAGAAAAATGACTACACG | SEQ ID NO.: 306 |
| JAB8 | F CACGTCACCCGCTTTCTCTTG | SEQ ID NO.: 307 |
| | R GGTGAGTGTAACACCTGTGTGCG | SEQ ID NO.: 308 |
| BMS2684 | F CCAAGGTCATTGTTGCAGC | SEQ ID NO.: 309 |
| | R TGGGGATTTGCTTCTCAGTC | SEQ ID NO.: 310 |
| DIK1106 | F CAAGAGTCAGACATGACTTAGTGAC | SEQ ID NO.: 311 |
| | R TCTACCTTTTGATAGCGTGAGC | SEQ ID NO.: 312 |
| INRA145 | F TAATAAAACTGGTCCCTCTGGC | SEQ ID NO.: 313 |
| | R TGCTGGCTCTCCAGTATGC | SEQ ID NO.: 314 |
| IDVGA-10 | F TCTCCTGGCTACAGGGCTAA | SEQ ID NO.: 315 |
| | R CCCACTGGCCTAGAACCC | SEQ ID NO.: 316 |
| DIK4850 | F AGGGGCGAAGTGAGGATTA | SEQ ID NO.: 317 |
| | R TTGCATGGTTCTGCAGATGT | SEQ ID NO.: 318 |
| DIK2768 | F AGCCTTCCCAGTACCTGTCA | SEQ ID NO.: 319 |
| | R TAAGGGAGCTCAAAACCACA | SEQ ID NO.: 320 |
| ILSTS027 | F GGTGTGTTGGTTAAGACTGG | SEQ ID NO.: 321 |
| | R GAATCATAGACCTGACTTCC | SEQ ID NO.: 322 |
| BMS812 | F TGGACAGGACTGAGTATGCA | SEQ ID NO.: 323 |
| | R AGGTATCCAACTAACACAGCCA | SEQ ID NO.: 324 |
| BMS2076 | F AGCACCTGTACCATCTGTTCC | SEQ ID NO.: 325 |
| | R TCCATAGGCTCACAAAGAGTTG | SEQ ID NO.: 326 |
| BL1095 | F TCCCTCTACCATATATTTCCCC | SEQ ID NO.: 327 |
| | R CATTAGCATGGAAAAACCTCTG | SEQ ID NO.: 328 |
| BMS820 | F CCACTACTTGCCTCAGGGAG | SEQ ID NO.: 329 |
| | R ACAGGACTCTCAAGCATCAGC | SEQ ID NO.: 330 |
| BMS927 | F GATGATCCACCATAACTACCAGA | SEQ ID NO.: 331 |
| | R TGGCTCTCAAAGGTCATTGT | SEQ ID NO.: 332 |
| BMS429 | F TACATTAACCCCAAAATTAAATGC | SEQ ID NO.: 333 |
| | R CCCTTGATTTCTCTCATGAGTATT | SEQ ID NO.: 334 |
| BTA18: | | |
| IDVGA-31 | F CCTTGAGATGAATGTTTGAGGATG | SEQ ID NO.: 335 |
| | R AACGCAGCCAGCAGGGTCAGG | SEQ ID NO.: 336 |
| BMS1355 | F TAAAACCCCAAAAAGAACCC | SEQ ID NO.: 337 |
| | R ATATTTGCGACATTGGATGAA | SEQ ID NO.: 338 |
| BMS1322 | F TGATGCTGATTGATTTTGTGTG | SEQ ID NO.: 339 |
| | R TATCTTTGCTCACTCTTTCCCC | SEQ ID NO.: 340 |
| TEXAN-10 | F TGTGGCTAGGTTCAAGCTCC | SEQ ID NO.: 341 |
| | R TCTCTTCTGGTGCATCCATTG | SEQ ID NO.: 342 |
| BMS2213 | F ATGGGCAGCTTAGGGATTG | SEQ ID NO.: 343 |
| | R CTTCAAGAGCCTTCAGTGGG | SEQ ID NO.: 344 |
| INRA121 | F GGAAACCCATTGGAGGATTTG | SEQ ID NO.: 345 |
| | R CTTCACTATTCCCCACAAAGC | SEQ ID NO.: 346 |
| BR4406 | F TACCTACCAGTTTTCCAGCACC | SEQ ID NO.: 347 |
| | R AGAAGAGCCTGGAGGGCTAC | SEQ ID NO.: 348 |
| BMS2554 | F GGGCTGTAAAGAGTAGGACACA | SEQ ID NO.: 349 |
| | R ATCATCTGCTTCCAGTCACAG | SEQ ID NO.: 350 |
| MNB-27 | F GAGTAAATAAAGCTGCATGATGTC | SEQ ID NO.: 351 |
| | R GGATCAGGAGATTTCAACACAG | SEQ ID NO.: 352 |
| BM7109 | F CAGGTAAAAGAGCGGCTTTG | SEQ ID NO.: 353 |
| | R GAGCTTCATGCCCTAGAAGG | SEQ ID NO.: 354 |
| INRA063 | F ATTTGCACAAGCTAAATCTAACC | SEQ ID NO.: 355 |
| | R AAACCACAGAAATGCTTGGAAG | SEQ ID NO.: 356 |
| ILSTS002 | F TCTATACACATGTGCTGTGC | SEQ ID NO.: 357 |
| | R CTTAGGGGTGAAGTGACACG | SEQ ID NO.: 358 |
| BMS2639 | F ATATCGTTTTCAGATTTCTTTTGC | SEQ ID NO.: 359 |
| | R GAGAGATAAATTGGGAGTTTGAGA | SEQ ID NO.: 360 |
| DIK4960 | F CGCAACTTCCAAGTCCATCT | SEQ ID NO.: 361 |
| | R GGACACCTTCCTGTCCTCAA | SEQ ID NO.: 362 |
| DIK4849 | F CCATCTTCCCCCATTGTGTA | SEQ ID NO.: 363 |
| | R CCCCTCTTCATCTCAAAACA | SEQ ID NO.: 364 |
| BMON117 | F TAGGGCCGTGATACTGTGT | SEQ ID NO.: 365 |
| | R CTCTACCATCCAGCACCCTAAT | SEQ ID NO.: 366 |
| DIK4232 | F TTGTGAGGTAAAGGGACATGA | SEQ ID NO.: 367 |
| | R GCCAGATTTGCCAACTGTTT | SEQ ID NO.: 368 |
| BMS2785 | F ACAAACCTGTGCGCCTTG | SEQ ID NO.: 369 |
| | R GGCAATCAGTCGGACACAC | SEQ ID NO.: 370 |
| DIK4569 | F TCCCCCTAAGGCTCAGAGTT | SEQ ID NO.: 371 |
| | R CTAACTTCCCCTTCGGAACC | SEQ ID NO.: 372 |
| BM2078 | F CCCAAAAGAAGCCAGGAAG | SEQ ID NO.: 373 |
| | R TCAGAGTTTGGGGTCCTCAG | SEQ ID NO.: 374 |
| BM6507 | F ACTTAGCACAATGCCCTCTAGG | SEQ ID NO.: 375 |
| | R ATGTTATTCCATCAGGAGGAGC | SEQ ID NO.: 376 |
| TGLA227 | F CGAATTCCAAATCTGTTAATTTGCT | SEQ ID NO.: 377 |
| | R ACAGACAGAAACTCAATGAAAGCA | SEQ ID NO.: 378 |
| DIK4013 | F GAAATTTGTGACCCCTGCAT | SEQ ID NO.: 379 |
| | R CTAAAGCTCTGCCTCCCAAG | SEQ ID NO.: 380 |
| BTA19: | | |
| BM9202 | F TCTATGAAGACTTTCAGGACCTTC | SEQ ID NO.: 381 |
| | R GCATCCCGGTCTCCTATG | SEQ ID NO.: 382 |
| BMS745 | F TAGGGACTTGTTACCCGTGG | SEQ ID NO.: 383 |
| | R TGCAAGCTGTGAGGAGGAG | SEQ ID NO.: 384 |
| BP20 | F TCTGTGGGTGAACAAGCAAG | SEQ ID NO.: 385 |
| | R GGCTCCCTAAAGACCCACTC | SEQ ID NO.: 386 |
| IDVGA-46 | F AAATCCTTTCAAGTATGTTTTCA | SEQ ID NO.: 387 |
| | R ACTCACTCCAGTATTCTTGTCTG | SEQ ID NO.: 388 |
| BMS2389 | F AATGTTAGGTTTACATGCAGCC | SEQ ID NO.: 389 |
| | R AGGCAATAGGATCTCCACTAGC | SEQ ID NO.: 390 |
| CSSM065 | F TTCCTGCTTGGTGAAACTTTGAAC | SEQ ID NO.: 391 |
| | R CAACTCAAAGCTTCAACAGCAGCC | SEQ ID NO.: 392 |
| ETH3 | F GAACCTGCCTCTCCTGCATTGG | SEQ ID NO.: 393 |
| | R ACTCTGCCTGTGGCCAAGTAGG | SEQ ID NO.: 394 |
| BMS601 | F CACTAGGACGATGCTCTCAGG | SEQ ID NO.: 395 |
| | R TCACAAGAGCAATGACGAGG | SEQ ID NO.: 396 |
| BTA20: | | |
| BM3517 | F GTGTGTTGGCATCTGGACTG | SEQ ID NO.: 397 |
| | R TGTCAAATTCTATGCAGGATGG | SEQ ID NO.: 398 |
| HEL12 | F GCATTAGGTTCTCCAGAGAA | SEQ ID NO.: 399 |
| | R CAGACTTGTCAGACTCCATA | SEQ ID NO.: 400 |
| BMS1282 | F ACTCTTCCACAGTTGGCCTG | SEQ ID NO.: 401 |
| | R CCTCCTTCCTCCAGAGCC | SEQ ID NO.: 402 |
| BMS1754 | F GCATTATTCTTTGTTCTTTGGG | SEQ ID NO.: 403 |
| | R GTTTCTGCTCCTGATCTCCTG | SEQ ID NO.: 404 |
| TGLA126 | F CTAATTTAGAATGAGAGAGGCTTCT | SEQ ID NO.: 405 |
| | R TTGGTCTCTATTCTCTGAATATTCC | SEQ ID NO.: 406 |
| BMS2361 | F ACACAACCCAAATGTTACCAA | SEQ ID NO.: 407 |
| | R ATTGTGCAGAGACCAAGTGC | SEQ ID NO.: 408 |
| AGLA29 | F AGGAAGCCGAGTGAGATATGTAAGC | SEQ ID NO.: 409 |
| | R TTACAGCCTGTGTGAATGTCCTCTA | SEQ ID NO.: 410 |
| BMS703 | F CAATGAGCTCAGATTGTTGCA | SEQ ID NO.: 411 |
| | R ATACATGTAGTCAAAAGGCTCATCC | SEQ ID NO.: 412 |
| BM5004 | F TCTGGAGTGAATGTTTCTGAGG | SEQ ID NO.: 413 |
| | R TTGTGATGAGCACCTGAAGG | SEQ ID NO.: 414 |
| UWCA26 | F TGGGGTCTAAAAGAGTCAGAG | SEQ ID NO.: 415 |
| | R TTCAAGTCTGCCTTTTGGTTTCGT | SEQ ID NO.: 416 |
| BTA21: | | |
| DIK5182 | F CGATGTAAAGGGCAGGTTCT | SEQ ID NO.: 417 |
| | R CTCTTAGAATCCTGTTTTAGGG | SEQ ID NO.: 418 |
| BMS1117 | F TGTGTGCTCTCTCACACATGC | SEQ ID NO.: 419 |
| | R AACCAAAGCAGGGATCAGG | SEQ ID NO.: 420 |
| RM151 | F CCCAGAGGTGACAACATTTCCAG | SEQ ID NO.: 421 |
| | R GATCCACCAAAAACCAGCTGGA | SEQ ID NO.: 422 |
| DIK2492 | F AATCCATCCATTCAGCCTTC | SEQ ID NO.: 423 |
| | R GAAATGACAGCCCACTCCAG | SEQ ID NO.: 424 |
| AGLA233 | F TGCAAACATCCACGTAGCATAAATA | SEQ ID NO.: 425 |
| | R GCATGAACAGCCAATAGTGTCATC | SEQ ID NO.: 426 |
| ILSTS095 | F GAAAGATGTTGCTAGTGGGG | SEQ ID NO.: 427 |
| | R ATTCTCCTGTGAACCTCTCC | SEQ ID NO.: 428 |
| DIK4602 | F GACTGCGACCAGGTCTTTTC | SEQ ID NO.: 429 |
| | R AGGCCCATACGCATTTGTTA | SEQ ID NO.: 430 |
| BM103 | F CTAGCTGCTGGCTACTTGGG | SEQ ID NO.: 431 |
| | R GGCTGCTCTGGGCTATTG | SEQ ID NO.: 432 |
| DIK4001 | F TTCTCCAACCCGGTTATGC | SEQ ID NO.: 433 |
| | R CTGATTGGTCACTCCATCCA | SEQ ID NO.: 434 |
| IDVGA-45 | F GTGGTGGCAAAGAGTCAGA | SEQ ID NO.: 435 |
| | R AACAGCCCTGATTTCCATA | SEQ ID NO.: 436 |
| DIK2481 | F CCGTGTTTGTCTTCCTCTGA | SEQ ID NO.: 437 |
| | R TGACAGCAGCCAAGATATGG | SEQ ID NO.: 438 |
| INRA103 | F TTGTCCAGCCCAGCATTTAGC | SEQ ID NO.: 439 |
| | R GGAGAAGACTTATGGGAGC | SEQ ID NO.: 440 |
| BMS2815 | F TGATATTCAAACTCAATGAACCC | SEQ ID NO.: 441 |
| | R CTTGCATATGCTCATCATTATCA | SEQ ID NO.: 442 |
| DIK2842 | F GGATTTTAGCTGCCATTGCT | SEQ ID NO.: 443 |
| | R AATCCCATGGACAGAAAAGC | SEQ ID NO.: 444 |
| DIK3036 | F TGTGTGGCTTTAGCACAT | SEQ ID NO.: 445 |
| | R CAGAAAGGGAAATCACATCC | SEQ ID NO.: 446 |
| DIK4391 | F CCCTTCCAATAGGCAAATCTC | SEQ ID NO.: 447 |
| | R TCCAACAAGCTTTTCCTTCC | SEQ ID NO.: 448 |
| DIK2913 | F AACGTCCAGTCGCTTCAAAT | SEQ ID NO.: 449 |
| | R TCACACACCTGAACTCAAAGC | SEQ ID NO.: 450 |
| BM846 | F GACCACTGGACCACCAGG | SEQ ID NO.: 451 |
| | R CTGGTAAAAAGCAATGATGCC | SEQ ID NO.: 452 |
| TGLA122 | F CCCTCCTCCAGGTAAATCAGC | SEQ ID NO.: 453 |
| | R AATCACATGGCAAATAAGTACATAC | SEQ ID NO.: 454 |
| ILSTS054 | F GAGGATCTTGATTTTGATGTCC | SEQ ID NO.: 455 |
| | R AGGGCCACTATGGTACTTCC | SEQ ID NO.: 456 |
| BMS743 | F AGCTACCCTGGTATACAACACG | SEQ ID NO.: 457 |
| | R GCTCTGAAATTCTGGCAGTG | SEQ ID NO.: 458 |
| IDVGA-30 | F GCATCTGGGAGCCTCGTATCTC | SEQ ID NO.: 459 |
| | R TTGTAAAACTCGGGGCATAAGCA | SEQ ID NO.: 460 |
| BTA22: | | |
| CSSM26 | F GACTTCTGCTTGTGGTTTCCAAGT | SEQ ID NO.: 461 |
| | R TTTTCCCATTATGGTTTATCCCAG | SEQ ID NO.: 462 |
| INRA026 | F TAGTTCCAATGAGACACGAACA | SEQ ID NO.: 463 |
| | R TAGGAGCACGGAGGTAAAACA | SEQ ID NO.: 464 |
| BM1558 | F TGAGGAAAGCCTTGGCAG | SEQ ID NO.: 465 |
| | R ACTGGGCCTAGCTCCTTCTC | SEQ ID NO.: 466 |
| BM3628 | F CTGAGATGGACTCAGGGAGG | SEQ ID NO.: 467 |
| | R GTTGGATTGGAAAGGTTAGGC | SEQ ID NO.: 468 |
| BMS875 | F TCCAGCTTGAATCCCTTCC | SEQ ID NO.: 469 |
| | R AAGCAAAGGCTGGGAACAC | SEQ ID NO.: 470 |
| BM4102 | F CCAAATTCCACTGTGCTGC | SEQ ID NO.: 471 |
| | R GAGCGGCCTATCAACCCTAC | SEQ ID NO.: 472 |
| BTA24: | | |
| BMS917 | F TAATGCCTCTGGAAGGTTGA | SEQ ID NO.: 473 |
| | R CAAGCTGGTTGTTCTTTTGC | SEQ ID NO.: 474 |
| BM7151 | F AAATGTCCACTGCTCAAAGATG | SEQ ID NO.: 475 |
| | R ACTTGGAGATAGAACTGGCAGG | SEQ ID NO.: 476 |
| BM226 | F ATTGCCTTGTCCGTGTATCC | SEQ ID NO.: 477 |
| | R CCGGCTGAATTGCTATAAGC | SEQ ID NO.: 478 |
| BMS2526 | F CAGGCTCCATGTTGGACAC | SEQ ID NO.: 479 |
| | R CATCAGGTTGGCAGAGTCG | SEQ ID NO.: 480 |
| TGLA351 | F GCACATCTGGTGGCCACATCAG | SEQ ID NO.: 481 |
| | R CTCTAGGGGATTTCAGTCTCAGT | SEQ ID NO.: 482 |
| BM7228 | F TTAAATCCTCAAGTAAAGGAAGGC | SEQ ID NO.: 483 |
| | R GCAAACCTAAGAATCCTCATTTC | SEQ ID NO.: 484 |
| CSSM23 | F CACTGGAGTGGGTTGCCATTGTCT | SEQ ID NO.: 485 |
| | R GTTCGCAATATGATCTCTGATTTG | SEQ ID NO.: 486 |
| BMS2270 | F CTGCGTTAACACCCCACC | SEQ ID NO.: 487 |
| | R GCAGGAAGGCTGATGCAC | SEQ ID NO.: 488 |
| ILSTS065 | F GCTGCAAAGAGTTGAACACC | SEQ ID NO.: 489 |
| | R AACTATTACAGGAGGCTCCC | SEQ ID NO.: 490 |
| BMS1862 | F GCACATGCAATCTTGAAAGG | SEQ ID NO.: 491 |
| | R ACCAGAGATGATGAAGAATCCC | SEQ ID NO.: 492 |
| BMS466 | F AGCAGAGGGCAAATGTTATG | SEQ ID NO.: 493 |
| | R GGATGTAAGAGGATGCAGACC | SEQ ID NO.: 494 |
| INRA090 | F GGTCATTTTCCATTATGACAGCAG | SEQ ID NO.: 495 |
| | R GGTGTTACCTTTTTTAGTCTCC | SEQ ID NO.: 496 |
| BMS1926 | F CAACTAGCTTCTCAATGCCTTT | SEQ ID NO.: 497 |
| | R TTCTCCCAATCTGTAACTGCA | SEQ ID NO.: 498 |
| BMS3024 | F CCAAACCAGTGTGACTGACA | SEQ ID NO.: 499 |
| | R TTGCTCATTTAACTTCATTACAACA | SEQ ID NO.: 500 |
| BTA25: | | |
| ILSTS102 | F CAGGACTGAGTAACTAAGGC | SEQ ID NO.: 501 |
| | R AGGAGACAGCTACAAACCCC | SEQ ID NO.: 502 |
| BMS2843 | F ATCCAAGGAGGTCCCAGG | SEQ ID NO.: 503 |
| | R TCCTCCAGTGGGAAATATGG | SEQ ID NO.: 504 |
| BM737 | F TGGGATAGACCACATTGGAA | SEQ ID NO.: 505 |
| | R GAATGCTGTTTGGGAGGGTA | SEQ ID NO.: 506 |
| ILSTS046 | F TAAAGTCCTGCAAGAGAAGG | SEQ ID NO.: 507 |
| | R TTTCTGTCTTGAGTCTCTCC | SEQ ID NO.: 508 |
| BMS1353 | F TTTCAGGACTAATAGGGCATGG | SEQ ID NO.: 509 |
| | R ATTCAGACCTGCCTGGTGAC | SEQ ID NO.: 510 |
| AF5 | F GCAGAAGGAAAAAGCAATGG | SEQ ID NO.: 511 |
| | R GATCCTGCGAGCCACAAG | SEQ ID NO.: 512 |
| BTA26: | | |
| BMS651 | F AATATGTGAAAACAAGTCAAAGCA | SEQ ID NO.: 513 |
| | R CCTGGCAAGCAACAGTTAAT | SEQ ID NO.: 514 |
| HEL11 | F CTTTGTGGAAGGCTAAGATG | SEQ ID NO.: 515 |
| | R TCCCACATGATCTATGGTGC | SEQ ID NO.: 516 |
| BMS332 | F GACAAAACCCTTTTAGCACAGG | SEQ ID NO.: 517 |
| | R AATTGCATGGAAAGTTCTCAGC | SEQ ID NO.: 518 |
| RM026 | F TTGTACATTTCTGTCAATGCCTT | SEQ ID NO.: 519 |
| | R ACAATGTCATTGGTCAATTCATT | SEQ ID NO.: 520 |
| BM9284 | F AGGTGCTGGAATGGCAAC | SEQ ID NO.: 521 |
| | R TGTGATTTTGGTCTTCCTTGC | SEQ ID NO.: 522 |
| RME40 | F TCTGTGAGCATGTGCAGAAT | SEQ ID NO.: 523 |
| | R CTCACAGGTAAATTTGGGTGAT | SEQ ID NO.: 524 |
| IDVGA-59 | F AACCCAAATATCCATCAATAG | SEQ ID NO.: 525 |
| | R CAGTCCCTCAACCCTCTTTTC | SEQ ID NO.: 526 |
| BMS882 | F TAGTGTCCACCAGAGACCCC | SEQ ID NO.: 527 |
| | R CCAAAGACACAGTTTAAAGGGC | SEQ ID NO.: 528 |
| BM804 | F CCAGCATCAACTGTCAGAGC | SEQ ID NO.: 529 |
| | R GGCAGATTCTTTGCCTTCTG | SEQ ID NO.: 530 |
| BM7237 | F CCAGCATCAACTGTCAGAGC | SEQ ID NO.: 531 |
| | R GGCAGATTCTTTGCCTTCTG | SEQ ID NO.: 532 |
| BTA28: | | |
| BMC6020 | F ATTGCATGTAGCTCTTGGGG | SEQ ID NO.: 533 |
| | R AAGTGGGTGGCTTCAACACT | SEQ ID NO.: 534 |
| ETH1112 | F AGTGGATCCTGCATGTTATGCCG | SEQ ID NO.: 535 |
| | R CCAGACGGACCTTTGTGGGCAA | SEQ ID NO.: 536 |
| BL25 | F AACAGTGGCAATGGAAGTGG | SEQ ID NO.: 537 |
| | R AGTCAGGATCTAGTGGGTGAGTG | SEQ ID NO.: 538 |
| DIK2955 | F CATTGAACACTGAAAGGAAAGC | SEQ ID NO.: 539 |
| | R TCACAAGGGCTTTGAAGTGA | SEQ ID NO.: 540 |
| BMS2608 | F GACTAAGCATATGAACCTGGGC | SEQ ID NO.: 541 |
| | R CTGCCCCTTGTCATCTCATC | SEQ ID NO.: 542 |
| BMS2658 | F TCCCTGGACTTCTTGCAGAG | SEQ ID NO.: 543 |
| | R CTGGCCCCAGACACAATC | SEQ ID NO.: 544 |
| DIK713 | F CACTTTGCTGTGGACCTGAA | SEQ ID NO.: 545 |
| | R ACCCAGGAACTGAACCCAT | SEQ ID NO.: 546 |
| BMS1714 | F TTTATCCCAAGAGGTTCCACC | SEQ ID NO.: 547 |
| | R AGGTGCTTGCAGTGAATCTG | SEQ ID NO.: 548 |
| DIK5056 | F CCACCAGGCTAATGGGTAAA | SEQ ID NO.: 549 |
| | R TGGTGTTGCATCTGCATTCT | SEQ ID NO.: 550 |
| DIK5323 | F CTGGGAAGCCTTTTGATCTG | SEQ ID NO.: 551 |
| | R ATGGACCAGATGGTGGAAAT | SEQ ID NO.: 552 |
| DIK4862 | F CTTTCCCATCCTTTCACCAA | SEQ ID NO.: 553 |
| | R AAGTAGGGTGTGTGGGGGTA | SEQ ID NO.: 554 |
| BMC2208 | F GTTGAGCAGGGGGTAACAAG | SEQ ID NO.: 555 |
| | R ACGAGTCCCTGCTGCTCTAC | SEQ ID NO.: 556 |

0.5 μl PCR-product is added to 9.5 μl formamide and analysed on an ABI-3730XL sequencing Instrument (Applied Biosystems Inc.).

### Phenotype Data

The calving traits considered were stillbirth (SB), calving difficulty (CD) and the size of calf at birth (CS) after first calving. The traits were assessed both as a “direct’ effect (D) of the sire in the calf and as a “maternal” effect (M) of the sire in the mother of the calf, giving a total of 6 traits for the QTL analysis. Breeding values for each trait were obtained from the Danish Agricultural Advisory Service database. The breeding values were obtained from the routine breeding value estimation procedure by the exception that information from correlated traits and pedigree information were ignored.

### Statistical Analysis

The calving traits were analyzed using the linear regression mapping procedure of Haley & Knott (1992). Significant QTL were found by using permutation tests developed by Churchill & Doerge (1994). In this procedure traits and chromosomes were analyzed separately and tested for the presence of a single QTL affecting a particular trait. If the test: (1) exceeds the 5% chromosome-wise significance threshold and (2) the QTL-region affecting two or more traits, then the QTL is retained for further characterization. The variance component QTL mapping approach was used to test if it is a single pleiotropic QTL affecting two traits or two linked QTL affecting different traits. The QTL is modeled as a random effect in a bivariate linear mixed model that adjusts for polygenenic and overall trait means. The IBD matrices were computed using a recursive algorithm (Sørensen et al., 2003, Wang et al., 1995), conditional on the most likely marker linkage phase in the sire. The IBD matrices were computed for every 2 cM along the chromosomes and used in the subsequent variance component estimation procedure.

Baysian information criterion (BIC) and correlation between the QTL (r_{q}) were used to compare the pleiotropic and linked model.

### Example 1

The chromosome-wise regression test (table 24) showed a total of 27 significant QTL for calving traits in first lactation on 17 different chromosomes. 15 of the QTL were related to direct calving ease and 12 QTL was related to the maternal effects.

Average number of informative markers per grandsire family varied from 3.0 (BTA25) to 8.5 (BTA3) informative markers per chromosome.

TABLE 24
Chromosome wise regressions analysis across families for calving traits after first
calving. QTL are shown for traits that exceed 5% chromosome wise threshold level.
Numbers of segregating families are shown in brackets for each trait and chromosome.

| | Inform. | | | | | | |
|---|---|---|---|---|---|---|---|
| BTA | Level | D_CD | D_SB | D_CS | M_CD | M_SB | M_CS |
| BTA3 | 8.5^{a} (34)^{b} | | 0.010^{c} (5)^{d} | | | | |
| BTA4 | 5.0 (19) | 0.023 (3) | | | | | |
| BTA7 | 6.4 (34) | | 0.003 (6) | 0.042 (5) | | | |
| BTA8 | 3.6 (34) | 0.042 (2) | | | | 0.030 (3) | |
| BTA9 | 6.0 (19) | | | | | | 0.027 (3) |
| BTA10 | 6.1 (34) | | | | 0.035 (3) | | |
| BTA12 | 5.1 (19) | | 0.031 (1) | | | 0.028 (2) | |
| BTA15 | 6.5 (34) | | | 0.02 (3) | | | |
| BTA18 | 7.0 (34) | 0.010 (5) | 0.026 (4) | 0 (4) | | 0.015 (7) | |
| BTA19 | 5.2 (19) | | | 0.007 (3) | | | |
| BTA20 | 3.5 (19) | | | 0.005 (5) | | | |
| BTA21 | 5.3 (34) | | | | 0.044 (2) | | |
| BTA22 | 4.1 (19) | | | 0.010 (2) | | | 0.029 (3) |
| BTA24 | 4.6 (19) | | | | | 0.041 (2) | |
| BTA25 | 3.0 (19) | 0.006 (2) | | 0.002 (4) | | | |
| BTA26 | 4.7 (34) | | 0.021 (3) | | | 0.00 (7) | |
| BTA28 | 3.5 (33) | | | | 0.025 (3) | 0.045 (0) | |

D_CD: direct calving difficulty,
D_SB: direct stillbirth,
D_CS: direct calf size,
M_CD: maternal calving difficulty,
M_SB: maternal stillbirth,
M_CS: maternal calf size.
^{a}numbers of informative markers,
^{b}number of analyzed grandsires,
^{c}p-values

Each QTL was detected significant in 0 to 7 Holstein families when the test was performed within family analysis. BTA 28 showed no significant families for M_SB, but four families were candidates to significance (p<0.10).

Seven chromosomes showed more than one significant QTL in the same region and were further examined for the presence of pleiotropic or linked QTL. Only BTA 18 showed more than two significant QTL.

### Example 2

Table 25 shows results of tests to distinguish between pleiotropic and linked QTL. Two regions (BTA 12, BTA25) indicate QTL with pleiotropic effects with strong correlations between the traits (close to 1 or −1). For BTA7 and BTA26 the linkage model is in favor with correlations closer to 0 and high BIC-values. The analysis on BTA22 and BTA28 could not clarify whether it is linked or pleiotropic QTL. BTA8 did not give useful results because the likelihood did not converge to a maximum. On BTA 18 there may be a pleiotropic QTL affecting all the direct calving traits and probably one QTL affecting maternal stillbirth (M_SB).

TABLE 25
Multi-trait analysis with pleiotropic and linked QTL models for calving
traits on BTA 7, 8, 12, 18, 22, 25, 26, and 28 where QTL were
identified for more than one calving trait in first lactation.

| | | | | No. | Bayes |
|---|---|---|---|---|---|
| BTA | | r_{q} | Dist (cM) | markers^{a} | factor^{b} |
| BTA7 | D_SB, D_CS | 0.35 | 26 | 1.15 | 0.3 |
| BTA8 | D_CD, M_SB | NC | 38 | 1.12 | NC |
| BTA12 | D_SB, M_SB | 0.99 | 4 | 0 | 27 |
| BTA18 | D_CD, D_SB | 0.87 | 0 | 0 | 27 |
| | D_CD, D_CS | 0.93 | 0 | 0 | 109848 |
| | D_CD, M_SB | 0.71 | 14 | 1.15 | 0.7 |
| | D_SB, D_CS | 0.95 | 0 | 0 | 1806411 |
| | D_SB, M_SB | NC | 14 | 1.15 | NC |
| | D_CS, M_SB | 0.49 | 14 | 1.15 | 0.7 |
| BTA22 | D_CS, M_SB | 0.72 | 14 | 0.68 | 3.7 |
| BTA25 | D_CD, D_CS | 1.00 | 0 | 0 | 548 |
| BTA26 | D_SB, M_SB | 0.1 | 10 | 0.32 | 0.13 |
| BTA28 | M_CD, M_SB | 0.78 | 10 | 0.39 | 3.7 |

D_CD: direct calving difficulty,
D_SB: direct stillbirth,
D_CS: direct calf size,
M_CD: maternal calving difficulty,
M_SB: maternal stillbirth,
M_CS: maternal calf size.
^{a}average number of informative markers between QTL,
^{b}probability of a pleiotropic model over the linked model

Several QTL affecting both direct and maternal calving traits were identified. The QTL for D_CD on BTA8 confirmed the result in Ashwell et al (2003) and the QTL for direct and maternal stillbirth on BTA7 and BTA18 confirmed the results in Kuhn et al (2003). The multi-trait and multiple QTL variance component approach detected two pleiotropic QTL affecting both direct calving size and calving difficulties, and two pleiotropic QTL affecting both direct and maternal stillbirth. The identified QTL could have important implications for the Danish Holstein breeding program because of relative high economic weight in the combined selection index. In particular, QTL affecting survival and stillbirth without affecting calf size will be an efficient way to improve genetic progress for calving traits. More marker information is needed to get a more precise characterization of the QTL, before it can be used for effective selection purposes.

1. A method of determining calving characteristics in a bovine subject, comprising detecting in a sample from said bovine subject the presence or absence of at least one genetic marker that is linked to at least one trait indicative of increased risk of stillbirth and/or increased risk of calving difficulties and/or increased risk of non-desired calf size, wherein said at least one genetic marker is located on the bovine chromosome BTA3 in a region flanked by and including polymorphic microsatellite markers INRA006 and BM7225 and/or
BTA4 in the region flanked by and including polymorphic microsatellite markers BMS1788 and MGTG4B and/or,
BTA5 in the region flanked by and including polymorphic microsatellite markers BMS1095 and BM2830 and/or,
BTA7 in a region flanked by and including polymorphic microsatellite markers BM7160 and BL1043 and/or,
BTA8 in a region flanked by and including polymorphic microsatellite markers IDVGA-11 and BMS836 and/or,
BTA9 in a region flanked by and including polymorphic microsatellite markers BMS2151 and BMS1967 and/or,
BTA10 in a region flanked by and including polymorphic microsatellite markers DIK2658 and BMS2614 and/or,
BTA11 in the region flanked by and including polymorphic microsatellite markers BM716 and HEL13 and/or,
BTA12 in a region flanked by and including polymorphic microsatellite markers BMS410 and BMS2724 and/or,
BTA15 in a region flanked by and including polymorphic microsatellite markers BR3510 and BMS429 and/or,
BTA18 in a region flanked by and including polymorphic microsatellite markers IDVGA-31 and DIK4013 and/or,
BTA19 in a region flanked by and including polymorphic microsatellite markers BM9202 and BMS601 and/or,
BTA20 in a region flanked by and including polymorphic microsatellite markers BM3517 and UWCA26 and/or,
BTA21 in a region flanked by and including polymorphic microsatellite markers DIK5182 and IDVGA-30 and/or,
BTA22 in a region flanked by and including polymorphic microsatellite markers CSSM26 and BM4102 and/or,
BTA24 in a region flanked by and including polymorphic microsatellite markers BMS917 and BMS3024 and/or,
BTA25 in a region flanked by and including polymorphic microsatellite markers ILSTS102 and AF5 and/or,
BTA26 in a region flanked by and including polymorphic microsatellite markers BMS651 and BM7237 and/or,
BTA28 in a region flanked by and including polymorphic microsatellite markers, BMC6020 and BMC2208,
wherein the presence of said at least one genetic marker is indicative of calving characteristics of said bovine subject and/or off-spring therefrom.

2-62. (canceled)

63. The method according to claim 1, wherein the at least one genetic marker is located in the region of the bovine chromosome BTA3 in the region from about 17.1 to 101.8 cM, or between genetic markers INRA006 and BM7225.

64. The method according to claim 1, wherein the at least one genetic marker is located in the region of the bovine chromosome BTA4 in the region from about 12.5 to 112.8 cM, or between genetic markers BMS1788 and MGTG4B.

65. The method according to claim 1, wherein the at least one genetic marker is located in the region of the bovine chromosome BTA5 in the region from about 0.0 to 116.9 cM, or between genetic markers BMS1095 and BM2830.

66. The method according to claim 1, wherein the at least one genetic marker is located in the region of the bovine chromosome BTA7 in the region from about 0.0 to 135.6 cM, or between genetic markers BM7160 and BL1043.

67. The method according to claim 1, wherein the at least one genetic marker is located in the region of the bovine chromosome BTA8 in the region from about 11.3 to 122.9 cM, or between genetic markers IDVGA-11 and BMS836.

68. The method according to claim 1, wherein the at least one genetic marker is located in the region of the bovine chromosome BTA9 in the region from about 8.49 to 109.3 cM, or between genetic markers BMS2151 and BMS1967.

69. The method according to claim 1, wherein the at least one genetic marker is located in the region of the bovine chromosome BTA10 in the region from about 2.7 to 109.4 cM, or between genetic markers DIK2658 and BMS2614.

70. The method according to claim 1, wherein the at least one genetic marker is located in the region of the bovine chromosome BTA11 in the region from about 19.4 to 122.4 cM, or between genetic markers BM716 and HEL13.

71. The method according to claim 1, wherein the at least one genetic marker is located in the region of the bovine chromosome BTA12 in the region from about 0.0 to 109.0 cM, or between genetic markers BMS410 and BMS2724.

72. The method according to claim 1, wherein the at least one genetic marker is located in the region of the bovine chromosome BTA15 in the region from about 9.4 to 109.8 cM, or between genetic markers BR3510 and BMS429.

73. The method according to claim 1, wherein the at least one genetic marker is located in the region of the bovine chromosome BTA18 in the region from about 0.0 to 84.4 cM, or between genetic markers IDVGA-31 and DIK4013.

74. The method according to claim 1, wherein the at least one genetic marker is located in the region of the bovine chromosome BTA19 in the region from about 0.0 to 108.0 cM, or between genetic markers BM9202 and BMS601.

75. The method according to claim 1, wherein the at least one genetic marker is located in the region of the bovine chromosome BTA20 in the region from about 0.0 to 77.1 cM, or between genetic markers BM3517 and UWCA26.

76. The method according to claim 1, wherein the at least one genetic marker is located in the region of the bovine chromosome BTA21 in the region from about 5.5 to 76.8 cM, or between genetic markers DIK5182 and IDVGA-30.

77. The method according to claim 1, wherein the at least one genetic marker is located in the region of the bovine chromosome BTA22 in the region from about 0.0 to 82.9 cM, or between genetic markers CSSM26 and BM4102.

78. The method according to claim 1, wherein the at least one genetic marker is located in the region of the bovine chromosome BTA24 in the region from about 6.2 to 65.9 cM, or between genetic markers BMS917 and BMS3024.

79. The method according to claim 1, wherein the at least one genetic marker is located in the region of the bovine chromosome BTA25 in the region from about 7.2 to 61.7 cM, or between genetic markers ILSTS102 and AF5.

80. The method according to claim 1, wherein the at least one genetic marker is located in the region of the bovine chromosome BTA26 in the region from about 2.8 to 66.8 cM, or between genetic markers BMS651 and BM7237.

81. The method according to claim 1, wherein the at least one genetic marker is located in the region of the bovine chromosome BTA28 in the region from about 8.0 to 59.6 cM, or between genetic markers BMC6020 and BMC2208.

82. A diagnostic kit for use in detecting the presence or absence in a bovine subject of at least one genetic marker associated with bovine calving characteristics, comprising at least one oligonucleotide sequence selected from the group consisting of SEQ ID NO.: 1 to SEQ ID NO.: 558 and combinations thereof.


SEQUENCE LISTING (558 sequences)

SEQ ID NO: 1 - DNA, 25 bp, Artificial
  Primer
aggaatatctgtatcaacctcagtc

SEQ ID NO: 2 - DNA, 26 bp, Artificial
  Primer
ctgagctggggtgggagctataaata

SEQ ID NO: 3 - DNA, 20 bp, Artificial
  Primer
tgtagctccctggaggagaa

SEQ ID NO: 4 - DNA, 22 bp, Artificial
  Primer
gcaaatacaacccagtctggtg

SEQ ID NO: 5 - DNA, 20 bp, Artificial
  Primer
gtgacctggagaagttttcc

SEQ ID NO: 6 - DNA, 20 bp, Artificial
  Primer
accacgctctgacttgtagc

SEQ ID NO: 7 - DNA, 20 bp, Artificial
  Primer
cgtgctgcaactgagagttc

SEQ ID NO: 8 - DNA, 23 bp, Artificial
  Primer
gctgtatagcaaagtgacccagt

SEQ ID NO: 9 - DNA, 21 bp, Artificial
  Primer
agaacaaatgtgacactcaca

SEQ ID NO: 10 - DNA, 20 bp, Artificial
  Primer
gtgagtacaggcgctttctg

SEQ ID NO: 11 - DNA, 21 bp, Artificial
  Primer
ggaggatgaaggagtctttgg

SEQ ID NO: 12 - DNA, 20 bp, Artificial
  Primer
aatttaccacagtccaccgc

SEQ ID NO: 13 - DNA, 22 bp, Artificial
  Primer
aaagaattggacctgactgagc

SEQ ID NO: 14 - DNA, 20 bp, Artificial
  Primer
gctttcacttctgctggctt

SEQ ID NO: 15 - DNA, 22 bp, Artificial
  Primer
ggtcttcattggtgttttctcc

SEQ ID NO: 16 - DNA, 20 bp, Artificial
  Primer
gagctgccctagatgaggtg

SEQ ID NO: 17 - DNA, 20 bp, Artificial
  Primer
cctctgccatctttattccg

SEQ ID NO: 18 - DNA, 25 bp, Artificial
  Primer
aagatcaacttattcctcacagtgg

SEQ ID NO: 19 - DNA, 20 bp, Artificial
  Primer
tgaactttagggcagcatga

SEQ ID NO: 20 - DNA, 21 bp, Artificial
  Primer
aagactgagatgtggggaaaa

SEQ ID NO: 21 - DNA, 24 bp, Artificial
  Primer
ctggaggtgtgtgagccccattta

SEQ ID NO: 22 - DNA, 24 bp, Artificial
  Primer
ctaagagtcgaaggtgtgactagg

SEQ ID NO: 23 - DNA, 20 bp, Artificial
  Primer
aagacaaggactttcagccc

SEQ ID NO: 24 - DNA, 22 bp, Artificial
  Primer
aaagagtcggacattactgagc

SEQ ID NO: 25 - DNA, 20 bp, Artificial
  Primer
tgttttgatggaacacagcc

SEQ ID NO: 26 - DNA, 20 bp, Artificial
  Primer
tggatttagaccagggttgg

SEQ ID NO: 27 - DNA, 22 bp, Artificial
  Primer
ttttctactgcccaacaaagtg

SEQ ID NO: 28 - DNA, 22 bp, Artificial
  Primer
taggtaccatagcctagccaag

SEQ ID NO: 29 - DNA, 20 bp, Artificial
  Primer
tctagaggatccccgctgac

SEQ ID NO: 30 - DNA, 20 bp, Artificial
  Primer
agagagcaactccactgtgc

SEQ ID NO: 31 - DNA, 24 bp, Artificial
  Primer
tataatgccctctagatccactca

SEQ ID NO: 32 - DNA, 25 bp, Artificial
  Primer
atggaaaaataagatgtggtatgtg

SEQ ID NO: 33 - DNA, 21 bp, Artificial
  Primer
actccagttttctttcctggg

SEQ ID NO: 34 - DNA, 21 bp, Artificial
  Primer
tgccatgtagtagctgtgtgc

SEQ ID NO: 35 - DNA, 21 bp, Artificial
  Primer
gtagccatggagactggactg

SEQ ID NO: 36 - DNA, 21 bp, Artificial
  Primer
cattatcccctgtcacacacc

SEQ ID NO: 37 - DNA, 20 bp, Artificial
  Primer
aactggctccaaggtcaatg

SEQ ID NO: 38 - DNA, 20 bp, Artificial
  Primer
tcccctgtcacacacctgta

SEQ ID NO: 39 - DNA, 21 bp, Artificial
  Primer
tgcgatatttaatggatgtct

SEQ ID NO: 40 - DNA, 20 bp, Artificial
  Primer
ttcctttctccgaactgctc

SEQ ID NO: 41 - DNA, 22 bp, Artificial
  Primer
tccatcaagtatttgagtgcaa

SEQ ID NO: 42 - DNA, 22 bp, Artificial
  Primer
atagccctacccactgtttctg

SEQ ID NO: 43 - DNA, 20 bp, Artificial
  Primer
atgtttttcaggccaatcca

SEQ ID NO: 44 - DNA, 20 bp, Artificial
  Primer
tgccctgatttctcataccc

SEQ ID NO: 45 - DNA, 23 bp, Artificial
  Primer
ggtgttatgcattctctaggtgc

SEQ ID NO: 46 - DNA, 24 bp, Artificial
  Primer
aagagttagacatgactgagcacg

SEQ ID NO: 47 - DNA, 22 bp, Artificial
  Primer
acgtccagattcagatttcttg

SEQ ID NO: 48 - DNA, 20 bp, Artificial
  Primer
ggagaggaatcttgcaaagg

SEQ ID NO: 49 - DNA, 23 bp, Artificial
  Primer
caaagccataagaagcaattatg

SEQ ID NO: 50 - DNA, 24 bp, Artificial
  Primer
ccttctatagtgtggtgactaccc

SEQ ID NO: 51 - DNA, 25 bp, Artificial
  Primer
gcacagtaataagagtgatggcaga

SEQ ID NO: 52 - DNA, 25 bp, Artificial
  Primer
tggagaagatttggctgtgtaccca

SEQ ID NO: 53 - DNA, 22 bp, Artificial
  Primer
cttaactcattcacctcaactg

SEQ ID NO: 54 - DNA, 22 bp, Artificial
  Primer
agtgattgagcacattgcgcat

SEQ ID NO: 55 - DNA, 20 bp, Artificial
  Primer
gcattggcaagtggattctt

SEQ ID NO: 56 - DNA, 24 bp, Artificial
  Primer
aaggcaattaacacatacatcacc

SEQ ID NO: 57 - DNA, 20 bp, Artificial
  Primer
acttcccatccatccatcag

SEQ ID NO: 58 - DNA, 22 bp, Artificial
  Primer
cttccattctcagccatctagc

SEQ ID NO: 59 - DNA, 25 bp, Artificial
  Primer
tgagccatagaattaagattcaagc

SEQ ID NO: 60 - DNA, 25 bp, Artificial
  Primer
tttgttcctctttattttcttctgc

SEQ ID NO: 61 - DNA, 25 bp, Artificial
  Primer
gagcagcttctttctttctcatctt

SEQ ID NO: 62 - DNA, 25 bp, Artificial
  Primer
gctcttggaagcttattgtataaag

SEQ ID NO: 63 - DNA, 22 bp, Artificial
  Primer
agggattggtttatgctctctc

SEQ ID NO: 64 - DNA, 20 bp, Artificial
  Primer
gttgcagagtcggacatgac

SEQ ID NO: 65 - DNA, 20 bp, Artificial
  Primer
gcaactaagacccaaccaac

SEQ ID NO: 66 - DNA, 22 bp, Artificial
  Primer
actgatgtgctcaggtatgacg

SEQ ID NO: 67 - DNA, 21 bp, Artificial
  Primer
gctttggtacaccctttaagc

SEQ ID NO: 68 - DNA, 22 bp, Artificial
  Primer
gaacaaattcacaagggaaaac

SEQ ID NO: 69 - DNA, 22 bp, Artificial
  Primer
tttcactgtcatctccctagca

SEQ ID NO: 70 - DNA, 22 bp, Artificial
  Primer
atgtattcatgcacaccacaca

SEQ ID NO: 71 - DNA, 22 bp, Artificial
  Primer
aaaatcccttcataacagtgcc

SEQ ID NO: 72 - DNA, 20 bp, Artificial
  Primer
catcgtgaattccagggttc

SEQ ID NO: 73 - DNA, 20 bp, Artificial
  Primer
ccaaaaattctggcaccaat

SEQ ID NO: 74 - DNA, 20 bp, Artificial
  Primer
cctgggcttgtgactagcat

SEQ ID NO: 75 - DNA, 20 bp, Artificial
  Primer
aggaaggacaaggacattgc

SEQ ID NO: 76 - DNA, 21 bp, Artificial
  Primer
agagggtcaaaggcttaatgg

SEQ ID NO: 77 - DNA, 25 bp, Artificial
  Primer
gaaactcaacccaagacaactcaag

SEQ ID NO: 78 - DNA, 25 bp, Artificial
  Primer
atgactttattctccacctagcaga

SEQ ID NO: 79 - DNA, 20 bp, Artificial
  Primer
tgtgctggaggtgatagctg

SEQ ID NO: 80 - DNA, 22 bp, Artificial
  Primer
tgcaggaatatgagagctgaga

SEQ ID NO: 81 - DNA, 20 bp, Artificial
  Primer
agttggacctgccattgttc

SEQ ID NO: 82 - DNA, 19 bp, Artificial
  Primer
acttatgtgcgtgcgtgct

SEQ ID NO: 83 - DNA, 20 bp, Artificial
  Primer
gcaccagcagagaggacatt

SEQ ID NO: 84 - DNA, 20 bp, Artificial
  Primer
accggctattgtccatcttg

SEQ ID NO: 85 - DNA, 20 bp, Artificial
  Primer
cagacacgactaagcgacca

SEQ ID NO: 86 - DNA, 20 bp, Artificial
  Primer
cctacaataaagcacgggga

SEQ ID NO: 87 - DNA, 18 bp, Artificial
  Primer
gcctgcatgtgtctgtgg

SEQ ID NO: 88 - DNA, 20 bp, Artificial
  Primer
tctgtgtcggaataccctcc

SEQ ID NO: 89 - DNA, 22 bp, Artificial
  Primer
tgaattgtttctgcttcttgga

SEQ ID NO: 90 - DNA, 20 bp, Artificial
  Primer
tgcatgactcccctctctct

SEQ ID NO: 91 - DNA, 23 bp, Artificial
  Primer
gttcaggactggccctgctaaca

SEQ ID NO: 92 - DNA, 23 bp, Artificial
  Primer
cctccagcccactttctcttctc

SEQ ID NO: 93 - DNA, 24 bp, Artificial
  Primer
tctctctaatggagttggtttttg

SEQ ID NO: 94 - DNA, 24 bp, Artificial
  Primer
atatcccactgaggataagaattc

SEQ ID NO: 95 - DNA, 24 bp, Artificial
  Primer
gagtagaacacaactgaggacaca

SEQ ID NO: 96 - DNA, 20 bp, Artificial
  Primer
caatgctgtgggtactgagg

SEQ ID NO: 97 - DNA, 20 bp, Artificial
  Primer
ggtttcctcaggacatggtg

SEQ ID NO: 98 - DNA, 20 bp, Artificial
  Primer
cagtccatgaggttgcagaa

SEQ ID NO: 99 - DNA, 21 bp, Artificial
  Primer
gtaatgtagccttttgtgccg

SEQ ID NO: 100 - DNA, 22 bp, Artificial
  Primer
tcaccaacatgagatagtgtgc

SEQ ID NO: 101 - DNA, 21 bp, Artificial
  Primer
tggtttagcagagagcacatg

SEQ ID NO: 102 - DNA, 18 bp, Artificial
  Primer
gctcctagccctgcacac

SEQ ID NO: 103 - DNA, 23 bp, Artificial
  Primer
attgatgctttatgatcctcatg

SEQ ID NO: 104 - DNA, 20 bp, Artificial
  Primer
cccactaagagaggaggagg

SEQ ID NO: 105 - DNA, 22 bp, Artificial
  Primer
aatgggcgtataaacacagatg

SEQ ID NO: 106 - DNA, 20 bp, Artificial
  Primer
tgagtcctgtcaccatcagc

SEQ ID NO: 107 - DNA, 24 bp, Artificial
  Primer
tggatttttaaacacagaatgtgg

SEQ ID NO: 108 - DNA, 25 bp, Artificial
  Primer
tcagcttctctttaaatttctctgg

SEQ ID NO: 109 - DNA, 22 bp, Artificial
  Primer
agccagtttcttcaaatcaacc

SEQ ID NO: 110 - DNA, 20 bp, Artificial
  Primer
atggttccgcagagaaacag

SEQ ID NO: 111 - DNA, 23 bp, Artificial
  Primer
ccaagggaggaaaaataagttaa

SEQ ID NO: 112 - DNA, 22 bp, Artificial
  Primer
accagcagtaggttgaggttaa

SEQ ID NO: 113 - DNA, 20 bp, Artificial
  Primer
aaccttcacaggctccttcc

SEQ ID NO: 114 - DNA, 20 bp, Artificial
  Primer
cccatctcttgtgccaaatc

SEQ ID NO: 115 - DNA, 20 bp, Artificial
  Primer
catctgaatggccagaatga

SEQ ID NO: 116 - DNA, 20 bp, Artificial
  Primer
gtcccctgcatgtgtctctc

SEQ ID NO: 117 - DNA, 21 bp, Artificial
  Primer
acattggcttacgctcacact

SEQ ID NO: 118 - DNA, 20 bp, Artificial
  Primer
cctgtctgggtttgtttgct

SEQ ID NO: 119 - DNA, 20 bp, Artificial
  Primer
atggacagaacagcctgaca

SEQ ID NO: 120 - DNA, 20 bp, Artificial
  Primer
tggtgaactcagcctcactg

SEQ ID NO: 121 - DNA, 20 bp, Artificial
  Primer
gtgctggacatctgcaagtg

SEQ ID NO: 122 - DNA, 22 bp, Artificial
  Primer
acattcaggtctgtgatccatg

SEQ ID NO: 123 - DNA, 25 bp, Artificial
  Primer
actaataagaaattctgcatgtgtg

SEQ ID NO: 124 - DNA, 23 bp, Artificial
  Primer
ccaccatgactcagaagtagttc

SEQ ID NO: 125 - DNA, 25 bp, Artificial
  Primer
taatcataagtcaaagtaacagttt

SEQ ID NO: 126 - DNA, 24 bp, Artificial
  Primer
gatctggacatacaaaagtattac

SEQ ID NO: 127 - DNA, 20 bp, Artificial
  Primer
ttacttttcgtgggccagag

SEQ ID NO: 128 - DNA, 20 bp, Artificial
  Primer
ggaactgtgccacatagcaa

SEQ ID NO: 129 - DNA, 20 bp, Artificial
  Primer
tcttggaaaggggaaaaagc

SEQ ID NO: 130 - DNA, 24 bp, Artificial
  Primer
tgcttcatagcacttatctcttca

SEQ ID NO: 131 - DNA, 25 bp, Artificial
  Primer
agtaaggcctgcagtatttatatcc

SEQ ID NO: 132 - DNA, 25 bp, Artificial
  Primer
aatctttccctagaacttacaaagg

SEQ ID NO: 133 - DNA, 24 bp, Artificial
  Primer
ctgaaacactctaaaagggtatgc

SEQ ID NO: 134 - DNA, 21 bp, Artificial
  Primer
atcccaacatccacccattcc

SEQ ID NO: 135 - DNA, 21 bp, Artificial
  Primer
gttctgaggtttgtaaagccc

SEQ ID NO: 136 - DNA, 21 bp, Artificial
  Primer
ggtgagctacaatccataggg

SEQ ID NO: 137 - DNA, 20 bp, Artificial
  Primer
aggaacccataggcagacac

SEQ ID NO: 138 - DNA, 20 bp, Artificial
  Primer
gcctggcaaagagaaaattc

SEQ ID NO: 139 - DNA, 20 bp, Artificial
  Primer
tctcaccctcacatggttca

SEQ ID NO: 140 - DNA, 20 bp, Artificial
  Primer
gtggagccaaggtgaaagaa

SEQ ID NO: 141 - DNA, 23 bp, Artificial
  Primer
ccagcagaagagaaagatactga

SEQ ID NO: 142 - DNA, 23 bp, Artificial
  Primer
agtggtagaacttccatctcaca

SEQ ID NO: 143 - DNA, 27 bp, Artificial
  Primer
aatccagtgtgtgaaagactaatccag

SEQ ID NO: 144 - DNA, 32 bp, Artificial
  Primer
gtagatcaagatatagaatatttttcaacacc

SEQ ID NO: 145 - DNA, 20 bp, Artificial
  Primer
ctcaatgacgtttggcttca

SEQ ID NO: 146 - DNA, 20 bp, Artificial
  Primer
ggtgcctgactccaattgat

SEQ ID NO: 147 - DNA, 20 bp, Artificial
  Primer
tgtctgtatttctgctgtgg

SEQ ID NO: 148 - DNA, 20 bp, Artificial
  Primer
acacggaagcgatctaaacg

SEQ ID NO: 149 - DNA, 18 bp, Artificial
  Primer
agtgccaaaaggaagcgc

SEQ ID NO: 150 - DNA, 19 bp, Artificial
  Primer
gacttgaccgttccacctg

SEQ ID NO: 151 - DNA, 21 bp, Artificial
  Primer
cctctgggtctatccatgttg

SEQ ID NO: 152 - DNA, 21 bp, Artificial
  Primer
tggatgaatgaagaagatgcc

SEQ ID NO: 153 - DNA, 21 bp, Artificial
  Primer
gacaagataggctttgcatga

SEQ ID NO: 154 - DNA, 25 bp, Artificial
  Primer
gatagaaatataccaggagctcaca

SEQ ID NO: 155 - DNA, 22 bp, Artificial
  Primer
accatctactgtgctatggctt

SEQ ID NO: 156 - DNA, 22 bp, Artificial
  Primer
gcagaaacacaatactcagtgc

SEQ ID NO: 157 - DNA, 21 bp, Artificial
  Primer
gggtagcctgttaaaatgcag

SEQ ID NO: 158 - DNA, 22 bp, Artificial
  Primer
cagtgctgacctctgaagtaag

SEQ ID NO: 159 - DNA, 22 bp, Artificial
  Primer
tgttcagtgcttgtcttagctg

SEQ ID NO: 160 - DNA, 21 bp, Artificial
  Primer
tcttcaaagccatcaatcatc

SEQ ID NO: 161 - DNA, 23 bp, Artificial
  Primer
aagctaactgatattctgccaca

SEQ ID NO: 162 - DNA, 19 bp, Artificial
  Primer
ttccctctcttccctctcc

SEQ ID NO: 163 - DNA, 22 bp, Artificial
  Primer
actggagacgactgaagcaacc

SEQ ID NO: 164 - DNA, 22 bp, Artificial
  Primer
gagtggctgttgctaaatttgg

SEQ ID NO: 165 - DNA, 23 bp, Artificial
  Primer
tacagtccatggggtcacaagag

SEQ ID NO: 166 - DNA, 25 bp, Artificial
  Primer
tctgaatctactccctcctcagagc

SEQ ID NO: 167 - DNA, 24 bp, Artificial
  Primer
tctctgtctctatcactatatggc

SEQ ID NO: 168 - DNA, 24 bp, Artificial
  Primer
ctgggcacctgaaactatcatcat

SEQ ID NO: 169 - DNA, 21 bp, Artificial
  Primer
gaaactcttttcactctgcgc

SEQ ID NO: 170 - DNA, 21 bp, Artificial
  Primer
gctcttagggattgcttcacc

SEQ ID NO: 171 - DNA, 23 bp, Artificial
  Primer
ccattaagaggaaattgtgttca

SEQ ID NO: 172 - DNA, 23 bp, Artificial
  Primer
atggagtcactgaaaggtactga

SEQ ID NO: 173 - DNA, 22 bp, Artificial
  Primer
gatcaccttgccactatttcct

SEQ ID NO: 174 - DNA, 21 bp, Artificial
  Primer
acatgacagccagctgctact

SEQ ID NO: 175 - DNA, 21 bp, Artificial
  Primer
taggctatgtactgaccatgc

SEQ ID NO: 176 - DNA, 21 bp, Artificial
  Primer
ctgaactgagatgactttggc

SEQ ID NO: 177 - DNA, 19 bp, Artificial
  Primer
cagctttccatcccctttc

SEQ ID NO: 178 - DNA, 20 bp, Artificial
  Primer
ctcccatcccaaacacagac

SEQ ID NO: 179 - DNA, 20 bp, Artificial
  Primer
ttgaccctgaaagatgtcca

SEQ ID NO: 180 - DNA, 21 bp, Artificial
  Primer
cacggtttatcagcttgggta

SEQ ID NO: 181 - DNA, 20 bp, Artificial
  Primer
actttcagttttgggctgac

SEQ ID NO: 182 - DNA, 20 bp, Artificial
  Primer
tgtcactaggtaaattggtg

SEQ ID NO: 183 - DNA, 24 bp, Artificial
  Primer
aaatggaggtaatgaaataaaata

SEQ ID NO: 184 - DNA, 21 bp, Artificial
  Primer
caaacccatggactgtaacct

SEQ ID NO: 185 - DNA, 21 bp, Artificial
  Primer
ttctgaatttgattcccaaca

SEQ ID NO: 186 - DNA, 22 bp, Artificial
  Primer
actgtttccttaaaagcttccc

SEQ ID NO: 187 - DNA, 20 bp, Artificial
  Primer
tgggtaagtgggaaaggatg

SEQ ID NO: 188 - DNA, 20 bp, Artificial
  Primer
ctcagccaggttgtcctctc

SEQ ID NO: 189 - DNA, 22 bp, Artificial
  Primer
ggaaagagtaggtgattccctg

SEQ ID NO: 190 - DNA, 22 bp, Artificial
  Primer
atttaattgtcatcccaggtga

SEQ ID NO: 191 - DNA, 20 bp, Artificial
  Primer
cctctgaaaccccagacttg

SEQ ID NO: 192 - DNA, 22 bp, Artificial
  Primer
aaaaacccaaaacaacacacaa

SEQ ID NO: 193 - DNA, 25 bp, Artificial
  Primer
ccttctctgaatttttgttgaaagc

SEQ ID NO: 194 - DNA, 22 bp, Artificial
  Primer
ggacagaagtgagtgactgaga

SEQ ID NO: 195 - DNA, 23 bp, Artificial
  Primer
catgatatttaccctgtgtgtgc

SEQ ID NO: 196 - DNA, 20 bp, Artificial
  Primer
gaggagctggagggctaaag

SEQ ID NO: 197 - DNA, 24 bp, Artificial
  Primer
ttggcacttactacctcatatgtt

SEQ ID NO: 198 - DNA, 22 bp, Artificial
  Primer
ttttctggatgttgagcctatt

SEQ ID NO: 199 - DNA, 20 bp, Artificial
  Primer
accttgggaatcaaggtcat

SEQ ID NO: 200 - DNA, 20 bp, Artificial
  Primer
cccagtagtccagtggctca

SEQ ID NO: 201 - DNA, 20 bp, Artificial
  Primer
aaagactgcttgcctgaagc

SEQ ID NO: 202 - DNA, 22 bp, Artificial
  Primer
caaccagtgatgctgtactctg

SEQ ID NO: 203 - DNA, 22 bp, Artificial
  Primer
tcaaaaagttggacatgactga

SEQ ID NO: 204 - DNA, 24 bp, Artificial
  Primer
aggttttcaaatgagagacttttc

SEQ ID NO: 205 - DNA, 20 bp, Artificial
  Primer
tcagtacactggccaccatg

SEQ ID NO: 206 - DNA, 20 bp, Artificial
  Primer
cactgcatgcttttccaaac

SEQ ID NO: 207 - DNA, 21 bp, Artificial
  Primer
gctcacaggttctgaggactc

SEQ ID NO: 208 - DNA, 22 bp, Artificial
  Primer
aacttgaagaaggaatgctgag

SEQ ID NO: 209 - DNA, 18 bp, Artificial
  Primer
gctctggtgacccaggtg

SEQ ID NO: 210 - DNA, 20 bp, Artificial
  Primer
ctggcaggagatgagaggag

SEQ ID NO: 211 - DNA, 22 bp, Artificial
  Primer
gggcagatgtgagtaattttcc

SEQ ID NO: 212 - DNA, 21 bp, Artificial
  Primer
aactgagctgtatggtggacg

SEQ ID NO: 213 - DNA, 20 bp, Artificial
  Primer
gcacattgggatctctcctg

SEQ ID NO: 214 - DNA, 20 bp, Artificial
  Primer
aaagtcccatcccacaatca

SEQ ID NO: 215 - DNA, 21 bp, Artificial
  Primer
tccttacaacacaccatgcaa

SEQ ID NO: 216 - DNA, 19 bp, Artificial
  Primer
cacacccaggcatccatac

SEQ ID NO: 217 - DNA, 24 bp, Artificial
  Primer
ttcatataagcagtttataaacgc

SEQ ID NO: 218 - DNA, 24 bp, Artificial
  Primer
ataggatctggtaacttacagatg

SEQ ID NO: 219 - DNA, 20 bp, Artificial
  Primer
ctcactccactgggcttctc

SEQ ID NO: 220 - DNA, 20 bp, Artificial
  Primer
tgtgttctcacctcgaccac

SEQ ID NO: 221 - DNA, 21 bp, Artificial
  Primer
tcatcttgggcataagacagg

SEQ ID NO: 222 - DNA, 22 bp, Artificial
  Primer
attgttcccagcatcttagagg

SEQ ID NO: 223 - DNA, 18 bp, Artificial
  Primer
cacccgtaccctcactgc

SEQ ID NO: 224 - DNA, 20 bp, Artificial
  Primer
tcacaaccctcttctcaccc

SEQ ID NO: 225 - DNA, 18 bp, Artificial
  Primer
tggcttgcaacactgcac

SEQ ID NO: 226 - DNA, 21 bp, Artificial
  Primer
cccacctacgactgggactta

SEQ ID NO: 227 - DNA, 21 bp, Artificial
  Primer
gcttcagttctgcttttcacc

SEQ ID NO: 228 - DNA, 21 bp, Artificial
  Primer
cttcagcatcttgattgttgc

SEQ ID NO: 229 - DNA, 20 bp, Artificial
  Primer
ctccaggtaagacaggccac

SEQ ID NO: 230 - DNA, 18 bp, Artificial
  Primer
cccgatctgtgtgtgggt

SEQ ID NO: 231 - DNA, 20 bp, Artificial
  Primer
tgtgggtttgatctctgagt

SEQ ID NO: 232 - DNA, 22 bp, Artificial
  Primer
tgtgtcctcctttgtggtagaa

SEQ ID NO: 233 - DNA, 22 bp, Artificial
  Primer
actaggaggccatataggaggc

SEQ ID NO: 234 - DNA, 20 bp, Artificial
  Primer
gagctcaaaacgagggacag

SEQ ID NO: 235 - DNA, 25 bp, Artificial
  Primer
atttctatgaagtagtcttctgact

SEQ ID NO: 236 - DNA, 25 bp, Artificial
  Primer
attttaaaactagtcacgagtgcct

SEQ ID NO: 237 - DNA, 22 bp, Artificial
  Primer
gatcctgcttatatttaaccac

SEQ ID NO: 238 - DNA, 22 bp, Artificial
  Primer
aaaattccatggagagagaaac

SEQ ID NO: 239 - DNA, 23 bp, Artificial
  Primer
tatgaactcacatggttaccaca

SEQ ID NO: 240 - DNA, 22 bp, Artificial
  Primer
ttgcccaaaaatagaccttaaa

SEQ ID NO: 241 - DNA, 20 bp, Artificial
  Primer
ggtattttgagaatgtgggc

SEQ ID NO: 242 - DNA, 20 bp, Artificial
  Primer
tctttgaccactacctatcc

SEQ ID NO: 243 - DNA, 20 bp, Artificial
  Primer
gtgcggaaaggaacagagtc

SEQ ID NO: 244 - DNA, 19 bp, Artificial
  Primer
aaagccggactggagtgtc

SEQ ID NO: 245 - DNA, 19 bp, Artificial
  Primer
aatgcgtgggacttgtttt

SEQ ID NO: 246 - DNA, 20 bp, Artificial
  Primer
caattgctgaagcagtcaca

SEQ ID NO: 247 - DNA, 21 bp, Artificial
  Primer
actttcttttcctgtggctcg

SEQ ID NO: 248 - DNA, 18 bp, Artificial
  Primer
cagagctggcaccagagg

SEQ ID NO: 249 - DNA, 21 bp, Artificial
  Primer
agtacttggcttgctttgctc

SEQ ID NO: 250 - DNA, 22 bp, Artificial
  Primer
ttaaatttccatctcaccctgg

SEQ ID NO: 251 - DNA, 18 bp, Artificial
  Primer
agagaggccaaagctggg

SEQ ID NO: 252 - DNA, 19 bp, Artificial
  Primer
tttccttgggcttcaggag

SEQ ID NO: 253 - DNA, 22 bp, Artificial
  Primer
ttctgtggttgaagagtgttcc

SEQ ID NO: 254 - DNA, 21 bp, Artificial
  Primer
caatggctaagaggtccagtg

SEQ ID NO: 255 - DNA, 23 bp, Artificial
  Primer
tccaaaagtttcgtgacatattg

SEQ ID NO: 256 - DNA, 20 bp, Artificial
  Primer
caccaggcttctctgttgaa

SEQ ID NO: 257 - DNA, 23 bp, Artificial
  Primer
tccaaaagtttcgtgacatattg

SEQ ID NO: 258 - DNA, 20 bp, Artificial
  Primer
caccaggcttctctgttgaa

SEQ ID NO: 259 - DNA, 21 bp, Artificial
  Primer
tcgcaaaaagttggacaagac

SEQ ID NO: 260 - DNA, 21 bp, Artificial
  Primer
ttagcagggtgcctgacactt

SEQ ID NO: 261 - DNA, 22 bp, Artificial
  Primer
ggtaaaatcctgcaaaacacag

SEQ ID NO: 262 - DNA, 22 bp, Artificial
  Primer
tgactgtatagactgaagcaac

SEQ ID NO: 263 - DNA, 22 bp, Artificial
  Primer
tggtatgtagttacagcagccc

SEQ ID NO: 264 - DNA, 22 bp, Artificial
  Primer
ccattgaaacagacatgaatgc

SEQ ID NO: 265 - DNA, 22 bp, Artificial
  Primer
gtgtctgtcaaaagatgaatgg

SEQ ID NO: 266 - DNA, 20 bp, Artificial
  Primer
gacaactgcttctcgttggg

SEQ ID NO: 267 - DNA, 20 bp, Artificial
  Primer
gagtattatgcttgggaggc

SEQ ID NO: 268 - DNA, 20 bp, Artificial
  Primer
agacaggatgggaagtcacc

SEQ ID NO: 269 - DNA, 21 bp, Artificial
  Primer
aaaggcttctatttgtggtgg

SEQ ID NO: 270 - DNA, 23 bp, Artificial
  Primer
ttgatgctttattgttttcctct

SEQ ID NO: 271 - DNA, 21 bp, Artificial
  Primer
ttctactctccagcctcctcc

SEQ ID NO: 272 - DNA, 20 bp, Artificial
  Primer
gttggctccaagagcaagtc

SEQ ID NO: 273 - DNA, 20 bp, Artificial
  Primer
actatggacatttggggcag

SEQ ID NO: 274 - DNA, 22 bp, Artificial
  Primer
agtaggtggagatcaaggatgc

SEQ ID NO: 275 - DNA, 22 bp, Artificial
  Primer
cagaccagtttctcagacaagc

SEQ ID NO: 276 - DNA, 22 bp, Artificial
  Primer
tcattcctgtgtcaatacagcc

SEQ ID NO: 277 - DNA, 23 bp, Artificial
  Primer
tttgagaacttttgtttctgagc

SEQ ID NO: 278 - DNA, 24 bp, Artificial
  Primer
ttattttgcttttctgattttgtg

SEQ ID NO: 279 - DNA, 25 bp, Artificial
  Primer
tgtatggctgaatgatattccattt

SEQ ID NO: 280 - DNA, 25 bp, Artificial
  Primer
ctactgacagatgattagataaaga

SEQ ID NO: 281 - DNA, 20 bp, Artificial
  Primer
tgccccatagtgtagtgctc

SEQ ID NO: 282 - DNA, 20 bp, Artificial
  Primer
gccagcagagaattgtagca

SEQ ID NO: 283 - DNA, 20 bp, Artificial
  Primer
ttctggcaaactattccacc

SEQ ID NO: 284 - DNA, 20 bp, Artificial
  Primer
catgaaagacacagatgacc

SEQ ID NO: 285 - DNA, 19 bp, Artificial
  Primer
atttttcccagcgcctctc

SEQ ID NO: 286 - DNA, 23 bp, Artificial
  Primer
aaacagaagactcaggaagacga

SEQ ID NO: 287 - DNA, 20 bp, Artificial
  Primer
taaggacttgagataaggag

SEQ ID NO: 288 - DNA, 20 bp, Artificial
  Primer
ccatctacctccatcttaac

SEQ ID NO: 289 - DNA, 20 bp, Artificial
  Primer
ggctgaaaagctgtggtgtt

SEQ ID NO: 290 - DNA, 22 bp, Artificial
  Primer
ttgccacatttaccttctttca

SEQ ID NO: 291 - DNA, 25 bp, Artificial
  Primer
ttctaatgtagagcaaagtgattga

SEQ ID NO: 292 - DNA, 20 bp, Artificial
  Primer
tgtaggagggacagattggg

SEQ ID NO: 293 - DNA, 21 bp, Artificial
  Primer
accagattggtggtagtggtg

SEQ ID NO: 294 - DNA, 18 bp, Artificial
  Primer
catgccgtggctaagacc

SEQ ID NO: 295 - DNA, 20 bp, Artificial
  Primer
tggagctaaatcaatgcgtg

SEQ ID NO: 296 - DNA, 21 bp, Artificial
  Primer
cccaatggccaattaagtacc

SEQ ID NO: 297 - DNA, 22 bp, Artificial
  Primer
ccttcatggaagaaattttgtg

SEQ ID NO: 298 - DNA, 21 bp, Artificial
  Primer
ggagttacagtccatgggttc

SEQ ID NO: 299 - DNA, 21 bp, Artificial
  Primer
ggctgatacacagagacatgc

SEQ ID NO: 300 - DNA, 21 bp, Artificial
  Primer
cctctctgccttctatcaggt

SEQ ID NO: 301 - DNA, 20 bp, Artificial
  Primer
gctggtgggttgtttaccac

SEQ ID NO: 302 - DNA, 20 bp, Artificial
  Primer
accccgtggactgtagtctg

SEQ ID NO: 303 - DNA, 23 bp, Artificial
  Primer
tgaagtaagtaagcacacaagca

SEQ ID NO: 304 - DNA, 22 bp, Artificial
  Primer
ttgatcatctttaggtccatcc

SEQ ID NO: 305 - DNA, 22 bp, Artificial
  Primer
acaggctacagtccatggggtt

SEQ ID NO: 306 - DNA, 25 bp, Artificial
  Primer
tatagaacagaaaaatgactacacg

SEQ ID NO: 307 - DNA, 21 bp, Artificial
  Primer
cacgtcacccgctttctcttg

SEQ ID NO: 308 - DNA, 23 bp, Artificial
  Primer
ggtgagtgtaacacctgtgtgcg

SEQ ID NO: 309 - DNA, 19 bp, Artificial
  Primer
ccaaggtcattgttgcagc

SEQ ID NO: 310 - DNA, 20 bp, Artificial
  Primer
tggggatttgcttctcagtc

SEQ ID NO: 311 - DNA, 25 bp, Artificial
  Primer
caagagtcagacatgacttagtgac

SEQ ID NO: 312 - DNA, 22 bp, Artificial
  Primer
tctaccttttgatagcgtgagc

SEQ ID NO: 313 - DNA, 22 bp, Artificial
  Primer
taataaaactggtccctctggc

SEQ ID NO: 314 - DNA, 19 bp, Artificial
  Primer
tgctggctctccagtatgc

SEQ ID NO: 315 - DNA, 20 bp, Artificial
  Primer
tctcctggctacagggctaa

SEQ ID NO: 316 - DNA, 18 bp, Artificial
  Primer
cccactggcctagaaccc

SEQ ID NO: 317 - DNA, 19 bp, Artificial
  Primer
aggggcgaagtgaggatta

SEQ ID NO: 318 - DNA, 20 bp, Artificial
  Primer
ttgcatggttctgcagatgt

SEQ ID NO: 319 - DNA, 20 bp, Artificial
  Primer
agccttcccagtacctgtca

SEQ ID NO: 320 - DNA, 20 bp, Artificial
  Primer
taagggagctcaaaaccaca

SEQ ID NO: 321 - DNA, 20 bp, Artificial
  Primer
ggtgtgttggttaagactgg

SEQ ID NO: 322 - DNA, 20 bp, Artificial
  Primer
gaatcatagacctgacttcc

SEQ ID NO: 323 - DNA, 20 bp, Artificial
  Primer
tggacaggactgagtatgca

SEQ ID NO: 324 - DNA, 22 bp, Artificial
  Primer
aggtatccaactaacacagcca

SEQ ID NO: 325 - DNA, 21 bp, Artificial
  Primer
agcacctgtaccatctgttcc

SEQ ID NO: 326 - DNA, 22 bp, Artificial
  Primer
tccataggctcacaaagagttg

SEQ ID NO: 327 - DNA, 22 bp, Artificial
  Primer
tccctctaccatatatttcccc

SEQ ID NO: 328 - DNA, 22 bp, Artificial
  Primer
cattagcatggaaaaacctctg

SEQ ID NO: 329 - DNA, 20 bp, Artificial
  Primer
ccactacttgcctcagggag

SEQ ID NO: 330 - DNA, 21 bp, Artificial
  Primer
acaggactctcaagcatcagc

SEQ ID NO: 331 - DNA, 23 bp, Artificial
  Primer
gatgatccaccataactaccaga

SEQ ID NO: 332 - DNA, 20 bp, Artificial
  Primer
tggctctcaaaggtcattgt

SEQ ID NO: 333 - DNA, 24 bp, Artificial
  Primer
tacattaaccccaaaattaaatgc

SEQ ID NO: 334 - DNA, 24 bp, Artificial
  Primer
cccttgatttctctcatgagtatt

SEQ ID NO: 335 - DNA, 24 bp, Artificial
  Primer
ccttgagatgaatgtttgaggatg

SEQ ID NO: 336 - DNA, 21 bp, Artificial
  Primer
aacgcagccagcagggtcagg

SEQ ID NO: 337 - DNA, 20 bp, Artificial
  Primer
taaaaccccaaaaagaaccc

SEQ ID NO: 338 - DNA, 21 bp, Artificial
  Primer
atatttgcgacattggatgaa

SEQ ID NO: 339 - DNA, 22 bp, Artificial
  Primer
tgatgctgattgattttgtgtg

SEQ ID NO: 340 - DNA, 22 bp, Artificial
  Primer
tatctttgctcactctttcccc

SEQ ID NO: 341 - DNA, 20 bp, Artificial
  Primer
tgtggctaggttcaagctcc

SEQ ID NO: 342 - DNA, 21 bp, Artificial
  Primer
tctcttctggtgcatccattg

SEQ ID NO: 343 - DNA, 19 bp, Artificial
  Primer
atgggcagcttagggattg

SEQ ID NO: 344 - DNA, 20 bp, Artificial
  Primer
cttcaagagccttcagtggg

SEQ ID NO: 345 - DNA, 21 bp, Artificial
  Primer
ggaaacccattggaggatttg

SEQ ID NO: 346 - DNA, 21 bp, Artificial
  Primer
cttcactattccccacaaagc

SEQ ID NO: 347 - DNA, 22 bp, Artificial
  Primer
tacctaccagttttccagcacc

SEQ ID NO: 348 - DNA, 20 bp, Artificial
  Primer
agaagagcctggagggctac

SEQ ID NO: 349 - DNA, 22 bp, Artificial
  Primer
gggctgtaaagagtaggacaca

SEQ ID NO: 350 - DNA, 21 bp, Artificial
  Primer
atcatctgcttccagtcacag

SEQ ID NO: 351 - DNA, 24 bp, Artificial
  Primer
gagtaaataaagctgcatgatgtc

SEQ ID NO: 352 - DNA, 22 bp, Artificial
  Primer
ggatcaggagatttcaacacag

SEQ ID NO: 353 - DNA, 20 bp, Artificial
  Primer
caggtaaaagagcggctttg

SEQ ID NO: 354 - DNA, 20 bp, Artificial
  Primer
cagcttcatgccctagaagg

SEQ ID NO: 355 - DNA, 23 bp, Artificial
  Primer
atttgcacaagctaaatctaacc

SEQ ID NO: 356 - DNA, 22 bp, Artificial
  Primer
aaaccacagaaatgcttggaag

SEQ ID NO: 357 - DNA, 20 bp, Artificial
  Primer
tctatacacatgtgctgtgc

SEQ ID NO: 358 - DNA, 20 bp, Artificial
  Primer
cttaggggtgaagtgacacg

SEQ ID NO: 359 - DNA, 24 bp, Artificial
  Primer
atatcgttttcagatttcttttgc

SEQ ID NO: 360 - DNA, 24 bp, Artificial
  Primer
gagagataaattgggagtttgaga

SEQ ID NO: 361 - DNA, 20 bp, Artificial
  Primer
cgcaacttccaagtccatct

SEQ ID NO: 362 - DNA, 20 bp, Artificial
  Primer
ggacaccttcctgtcctcaa

SEQ ID NO: 363 - DNA, 20 bp, Artificial
  Primer
ccatcttcccccattgtgta

SEQ ID NO: 364 - DNA, 20 bp, Artificial
  Primer
cccctcttcatctcaaaaca

SEQ ID NO: 365 - DNA, 19 bp, Artificial
  Primer
tagggccgtgatactgtgt

SEQ ID NO: 366 - DNA, 22 bp, Artificial
  Primer
ctctaccatccagcaccctaat

SEQ ID NO: 367 - DNA, 21 bp, Artificial
  Primer
ttgtgaggtaaagggacatga

SEQ ID NO: 368 - DNA, 20 bp, Artificial
  Primer
gccagatttgccaactgttt

SEQ ID NO: 369 - DNA, 18 bp, Artificial
  Primer
acaaacctgtgcgccttg

SEQ ID NO: 370 - DNA, 19 bp, Artificial
  Primer
ggcaatcagtcggacacac

SEQ ID NO: 371 - DNA, 20 bp, Artificial
  Primer
tccccctaaggctcagagtt

SEQ ID NO: 372 - DNA, 20 bp, Artificial
  Primer
ctaacttccccttcggaacc

SEQ ID NO: 373 - DNA, 19 bp, Artificial
  Primer
cccaaaagaagccaggaag

SEQ ID NO: 374 - DNA, 20 bp, Artificial
  Primer
tcagagtttggggtcctcag

SEQ ID NO: 375 - DNA, 22 bp, Artificial
  Primer
acttagcacaatgccctctagg

SEQ ID NO: 376 - DNA, 22 bp, Artificial
  Primer
atgttattccatcaggaggagc

SEQ ID NO: 377 - DNA, 25 bp, Artificial
  Primer
cgaattccaaatctgttaatttgct

SEQ ID NO: 378 - DNA, 24 bp, Artificial
  Primer
acagacagaaactcaatgaaagca

SEQ ID NO: 379 - DNA, 20 bp, Artificial
  Primer
gaaatttgtgacccctgcat

SEQ ID NO: 380 - DNA, 20 bp, Artificial
  Primer
ctaaagctctgcctcccaag

SEQ ID NO: 381 - DNA, 24 bp, Artificial
  Primer
tctatgaagactttcaggaccttc

SEQ ID NO: 382 - DNA, 18 bp, Artificial
  Primer
gcatcccggtctcctatg

SEQ ID NO: 383 - DNA, 20 bp, Artificial
  Primer
tagggacttgttacccgtgg

SEQ ID NO: 384 - DNA, 19 bp, Artificial
  Primer
tgcaagctgtgaggaggag

SEQ ID NO: 385 - DNA, 20 bp, Artificial
  Primer
tctgtgggtgaacaagcaag

SEQ ID NO: 386 - DNA, 20 bp, Artificial
  Primer
ggctccctaaagacccactc

SEQ ID NO: 387 - DNA, 23 bp, Artificial
  Primer
aaatcctttcaagtatgttttca

SEQ ID NO: 388 - DNA, 23 bp, Artificial
  Primer
actcactccagtattcttgtctg

SEQ ID NO: 389 - DNA, 22 bp, Artificial
  Primer
aatgttaggtttacatgcagcc

SEQ ID NO: 390 - DNA, 22 bp, Artificial
  Primer
aggcaataggatctccactagc

SEQ ID NO: 391 - DNA, 24 bp, Artificial
  Primer
ttcctgcttggtgaaactttgaac

SEQ ID NO: 392 - DNA, 24 bp, Artificial
  Primer
caactcaaagcttcaacagcagcc

SEQ ID NO: 393 - DNA, 22 bp, Artificial
  Primer
gaacctgcctctcctgcattgg

SEQ ID NO: 394 - DNA, 22 bp, Artificial
  Primer
actctgcctgtggccaagtagg

SEQ ID NO: 395 - DNA, 21 bp, Artificial
  Primer
cactaggacgatgctctcagg

SEQ ID NO: 396 - DNA, 20 bp, Artificial
  Primer
tcacaagagcaatgacgagg

SEQ ID NO: 397 - DNA, 20 bp, Artificial
  Primer
gtgtgttggcatctggactg

SEQ ID NO: 398 - DNA, 22 bp, Artificial
  Primer
tgtcaaattctatgcaggatgg

SEQ ID NO: 399 - DNA, 20 bp, Artificial
  Primer
gcattaggttctccagagaa

SEQ ID NO: 400 - DNA, 20 bp, Artificial
  Primer
cagacttgtcagactccata

SEQ ID NO: 401 - DNA, 20 bp, Artificial
  Primer
actcttccacagttggcctg

SEQ ID NO: 402 - DNA, 18 bp, Artificial
  Primer
cctccttcctccagagcc

SEQ ID NO: 403 - DNA, 22 bp, Artificial
  Primer
gcattattctttgttctttggg

SEQ ID NO: 404 - DNA, 21 bp, Artificial
  Primer
gtttctgctcctgatctcctg

SEQ ID NO: 405 - DNA, 25 bp, Artificial
  Primer
ctaatttagaatgagagaggcttct

SEQ ID NO: 406 - DNA, 25 bp, Artificial
  Primer
ttggtctctattctctgaatattcc

SEQ ID NO: 407 - DNA, 21 bp, Artificial
  Primer
acacaacccaaatgttaccaa

SEQ ID NO: 408 - DNA, 20 bp, Artificial
  Primer
attgtgcagagaccaagtgc

SEQ ID NO: 409 - DNA, 25 bp, Artificial
  Primer
aggaagccgagtgagatatgtaagc

SEQ ID NO: 410 - DNA, 25 bp, Artificial
  Primer
ttacagcctgtgtgaatgtcctcta

SEQ ID NO: 411 - DNA, 21 bp, Artificial
  Primer
caatgagctcagattgttgca

SEQ ID NO: 412 - DNA, 25 bp, Artificial
  Primer
atacatgtagtcaaaaggctcatcc

SEQ ID NO: 413 - DNA, 22 bp, Artificial
  Primer
tctggagtgaatgtttctgagg

SEQ ID NO: 414 - DNA, 20 bp, Artificial
  Primer
ttgtgatgagcacctgaagg

SEQ ID NO: 415 - DNA, 21 bp, Artificial
  Primer
tggggtctaaaagagtcagag

SEQ ID NO: 416 - DNA, 24 bp, Artificial
  Primer
ttcaagtctgccttttggtttcgt

SEQ ID NO: 417 - DNA, 20 bp, Artificial
  Primer
cgatgtaaagggcaggttct

SEQ ID NO: 418 - DNA, 22 bp, Artificial
  Primer
ctcttagaatcctgttttaggg

SEQ ID NO: 419 - DNA, 21 bp, Artificial
  Primer
tgtgtgctctctcacacatgc

SEQ ID NO: 420 - DNA, 19 bp, Artificial
  Primer
aaccaaagcagggatcagg

SEQ ID NO: 421 - DNA, 23 bp, Artificial
  Primer
cccagaggtgacaacatttccag

SEQ ID NO: 422 - DNA, 22 bp, Artificial
  Primer
gatccaccaaaaaccagctgga

SEQ ID NO: 423 - DNA, 20 bp, Artificial
  Primer
aatccatccattcagccttc

SEQ ID NO: 424 - DNA, 20 bp, Artificial
  Primer
gaaatgacagcccactccag

SEQ ID NO: 425 - DNA, 25 bp, Artificial
  Primer
tgcaaacatccacgtagcataaata

SEQ ID NO: 426 - DNA, 24 bp, Artificial
  Primer
gcatgaacagccaatagtgtcatc

SEQ ID NO: 427 - DNA, 20 bp, Artificial
  Primer
gaaagatgttgctagtgggg

SEQ ID NO: 428 - DNA, 20 bp, Artificial
  Primer
attctcctgtgaacctctcc

SEQ ID NO: 429 - DNA, 20 bp, Artificial
  Primer
gactgcgaccaggtcttttc

SEQ ID NO: 430 - DNA, 20 bp, Artificial
  Primer
aggcccatacgcatttgtta

SEQ ID NO: 431 - DNA, 20 bp, Artificial
  Primer
ctagctgctggctacttggg

SEQ ID NO: 432 - DNA, 18 bp, Artificial
  Primer
ggctgctctgggctattg

SEQ ID NO: 433 - DNA, 19 bp, Artificial
  Primer
ttctccaacccggttatgc

SEQ ID NO: 434 - DNA, 20 bp, Artificial
  Primer
ctgattggtcactccatcca

SEQ ID NO: 435 - DNA, 19 bp, Artificial
  Primer
gtggtggcaaagagtcaga

SEQ ID NO: 436 - DNA, 19 bp, Artificial
  Primer
aacagccctgatttccata

SEQ ID NO: 437 - DNA, 20 bp, Artificial
  Primer
ccgtgtttgtcttcctctga

SEQ ID NO: 438 - DNA, 20 bp, Artificial
  Primer
tgacagcagccaagatatgg

SEQ ID NO: 439 - DNA, 21 bp, Artificial
  Primer
ttgtccagcccagcatttagc

SEQ ID NO: 440 - DNA, 19 bp, Artificial
  Primer
ggagaagacttatgggagc

SEQ ID NO: 441 - DNA, 23 bp, Artificial
  Primer
tgatattcaaactcaatgaaccc

SEQ ID NO: 442 - DNA, 23 bp, Artificial
  Primer
cttgcatatgctcatcattatca

SEQ ID NO: 443 - DNA, 20 bp, Artificial
  Primer
ggattttagctgccattgct

SEQ ID NO: 444 - DNA, 20 bp, Artificial
  Primer
aatcccatggacagaaaagc

SEQ ID NO: 445 - DNA, 18 bp, Artificial
  Primer
tgtgtggctttagcacat

SEQ ID NO: 446 - DNA, 20 bp, Artificial
  Primer
cagaaagggaaatcacatcc

SEQ ID NO: 447 - DNA, 21 bp, Artificial
  Primer
cccttccaataggcaaatctc

SEQ ID NO: 448 - DNA, 20 bp, Artificial
  Primer
tccaacaagcttttccttcc

SEQ ID NO: 449 - DNA, 20 bp, Artificial
  Primer
aacgtccagtcgcttcaaat

SEQ ID NO: 450 - DNA, 21 bp, Artificial
  Primer
tcacacacctgaactcaaagc

SEQ ID NO: 451 - DNA, 18 bp, Artificial
  Primer
gaccactggaccaccagg

SEQ ID NO: 452 - DNA, 21 bp, Artificial
  Primer
ctggtaaaaagcaatgatgcc

SEQ ID NO: 453 - DNA, 21 bp, Artificial
  Primer
ccctcctccaggtaaatcagc

SEQ ID NO: 454 - DNA, 25 bp, Artificial
  Primer
aatcacatggcaaataagtacatac

SEQ ID NO: 455 - DNA, 22 bp, Artificial
  Primer
gaggatcttgattttgatgtcc

SEQ ID NO: 456 - DNA, 20 bp, Artificial
  Primer
agggccactatggtacttcc

SEQ ID NO: 457 - DNA, 22 bp, Artificial
  Primer
agctaccctggtatacaacacg

SEQ ID NO: 458 - DNA, 20 bp, Artificial
  Primer
gctctgaaattctggcagtg

SEQ ID NO: 459 - DNA, 22 bp, Artificial
  Primer
gcatctgggagcctcgtatctc

SEQ ID NO: 460 - DNA, 23 bp, Artificial
  Primer
ttgtaaaactcggggcataagca

SEQ ID NO: 461 - DNA, 24 bp, Artificial
  Primer
gacttctgcttgtggtttccaagt

SEQ ID NO: 462 - DNA, 24 bp, Artificial
  Primer
ttttcccattatggtttatcccag

SEQ ID NO: 463 - DNA, 22 bp, Artificial
  Primer
tagttccaatgagacacgaaca

SEQ ID NO: 464 - DNA, 21 bp, Artificial
  Primer
taggagcacggaggtaaaaca

SEQ ID NO: 465 - DNA, 18 bp, Artificial
  Primer
tgaggaaagccttggcag

SEQ ID NO: 466 - DNA, 20 bp, Artificial
  Primer
actgggcctagctccttctc

SEQ ID NO: 467 - DNA, 20 bp, Artificial
  Primer
ctgagatggactcagggagg

SEQ ID NO: 468 - DNA, 21 bp, Artificial
  Primer
gttggattggaaaggttaggc

SEQ ID NO: 469 - DNA, 19 bp, Artificial
  Primer
tccagcttgaatcccttcc

SEQ ID NO: 470 - DNA, 19 bp, Artificial
  Primer
aagcaaaggctgggaacac

SEQ ID NO: 471 - DNA, 19 bp, Artificial
  Primer
ccaaattccactgtgctgc

SEQ ID NO: 472 - DNA, 20 bp, Artificial
  Primer
gagcggcctatcaaccctac

SEQ ID NO: 473 - DNA, 20 bp, Artificial
  Primer
taatgcctctggaaggttga

SEQ ID NO: 474 - DNA, 20 bp, Artificial
  Primer
caagctggttgttcttttgc

SEQ ID NO: 475 - DNA, 22 bp, Artificial
  Primer
aaatgtccactgctcaaagatg

SEQ ID NO: 476 - DNA, 22 bp, Artificial
  Primer
acttggagatagaactggcagg

SEQ ID NO: 477 - DNA, 20 bp, Artificial
  Primer
attgccttgtccgtgtatcc

SEQ ID NO: 478 - DNA, 20 bp, Artificial
  Primer
ccggctgaattgctataagc

SEQ ID NO: 479 - DNA, 19 bp, Artificial
  Primer
caggctccatgttggacac

SEQ ID NO: 480 - DNA, 19 bp, Artificial
  Primer
catcaggttggcagagtcg

SEQ ID NO: 481 - DNA, 22 bp, Artificial
  Primer
gcacatctggtggccacatcag

SEQ ID NO: 482 - DNA, 23 bp, Artificial
  Primer
ctctaggggatttcagtctcagt

SEQ ID NO: 483 - DNA, 24 bp, Artificial
  Primer
ttaaatcctcaagtaaaggaaggc

SEQ ID NO: 484 - DNA, 23 bp, Artificial
  Primer
gcaaacctaagaatcctcatttc

SEQ ID NO: 485 - DNA, 24 bp, Artificial
  Primer
cactggagtgggttgccattgtct

SEQ ID NO: 486 - DNA, 24 bp, Artificial
  Primer
gttcgcaatatgatctctgatttg

SEQ ID NO: 487 - DNA, 18 bp, Artificial
  Primer
ctgcgttaacaccccacc

SEQ ID NO: 488 - DNA, 18 bp, Artificial
  Primer
gcaggaaggctgatgcac

SEQ ID NO: 489 - DNA, 20 bp, Artificial
  Primer
gctgcaaagagttgaacacc

SEQ ID NO: 490 - DNA, 20 bp, Artificial
  Primer
aactattacaggaggctccc

SEQ ID NO: 491 - DNA, 20 bp, Artificial
  Primer
gcacatgcaatcttgaaagg

SEQ ID NO: 492 - DNA, 22 bp, Artificial
  Primer
accagagatgatgaagaatccc

SEQ ID NO: 493 - DNA, 20 bp, Artificial
  Primer
agcagagggcaaatgttatg

SEQ ID NO: 494 - DNA, 21 bp, Artificial
  Primer
ggatgtaagaggatgcagacc

SEQ ID NO: 495 - DNA, 24 bp, Artificial
  Primer
ggtcattttccattatgacagcag

SEQ ID NO: 496 - DNA, 22 bp, Artificial
  Primer
ggtgttaccttttttagtctcc

SEQ ID NO: 497 - DNA, 22 bp, Artificial
  Primer
caactagcttctcaatgccttt

SEQ ID NO: 498 - DNA, 21 bp, Artificial
  Primer
ttctcccaatctgtaactgca

SEQ ID NO: 499 - DNA, 20 bp, Artificial
  Primer
ccaaaccagtgtgactgaca

SEQ ID NO: 500 - DNA, 25 bp, Artificial
  Primer
ttgctcatttaacttcattacaaca

SEQ ID NO: 501 - DNA, 20 bp, Artificial
  Primer
caggactgagtaactaaggc

SEQ ID NO: 502 - DNA, 20 bp, Artificial
  Primer
aggagacagctacaaacccc

SEQ ID NO: 503 - DNA, 18 bp, Artificial
  Primer
atccaaggaggtcccagg

SEQ ID NO: 504 - DNA, 20 bp, Artificial
  Primer
tcctccagtgggaaatatgg

SEQ ID NO: 505 - DNA, 20 bp, Artificial
  Primer
tgggatagaccacattggaa

SEQ ID NO: 506 - DNA, 20 bp, Artificial
  Primer
gaatgctgtttgggagggta

SEQ ID NO: 507 - DNA, 20 bp, Artificial
  Primer
taaagtcctgcaagagaagg

SEQ ID NO: 508 - DNA, 20 bp, Artificial
  Primer
tttctgtcttgagtctctcc

SEQ ID NO: 509 - DNA, 22 bp, Artificial
  Primer
tttcaggactaatagggcatgg

SEQ ID NO: 510 - DNA, 20 bp, Artificial
  Primer
attcagacctgcctggtgac

SEQ ID NO: 511 - DNA, 20 bp, Artificial
  Primer
gcagaaggaaaaagcaatgg

SEQ ID NO: 512 - DNA, 18 bp, Artificial
  Primer
gatcctgcgagccacaag

SEQ ID NO: 513 - DNA, 24 bp, Artificial
  Primer
aatatgtgaaaacaagtcaaagca

SEQ ID NO: 514 - DNA, 20 bp, Artificial
  Primer
cctggcaagcaacagttaat

SEQ ID NO: 515 - DNA, 20 bp, Artificial
  Primer
ctttgtggaaggctaagatg

SEQ ID NO: 516 - DNA, 20 bp, Artificial
  Primer
tcccacatgatctatggtgc

SEQ ID NO: 517 - DNA, 22 bp, Artificial
  Primer
gacaaaacccttttagcacagg

SEQ ID NO: 518 - DNA, 22 bp, Artificial
  Primer
aattgcatggaaagttctcagc

SEQ ID NO: 519 - DNA, 23 bp, Artificial
  Primer
ttgtacatttctgtcaatgcctt

SEQ ID NO: 520 - DNA, 23 bp, Artificial
  Primer
acaatgtcattggtcaattcatt

SEQ ID NO: 521 - DNA, 18 bp, Artificial
  Primer
aggtgctggaatggcaac

SEQ ID NO: 522 - DNA, 21 bp, Artificial
  Primer
tgtgattttggtcttccttgc

SEQ ID NO: 523 - DNA, 20 bp, Artificial
  Primer
tctgtgagcatgtgcagaat

SEQ ID NO: 524 - DNA, 22 bp, Artificial
  Primer
ctcacaggtaaatttgggtgat

SEQ ID NO: 525 - DNA, 21 bp, Artificial
  Primer
aacccaaatatccatcaatag

SEQ ID NO: 526 - DNA, 21 bp, Artificial
  Primer
cagtccctcaaccctcttttc

SEQ ID NO: 527 - DNA, 20 bp, Artificial
  Primer
tagtgtccaccagagacccc

SEQ ID NO: 528 - DNA, 22 bp, Artificial
  Primer
ccaaagacacagtttaaagggc

SEQ ID NO: 529 - DNA, 20 bp, Artificial
  Primer
ccagcatcaactgtcagagc

SEQ ID NO: 530 - DNA, 20 bp, Artificial
  Primer
ggcagattctttgccttctg

SEQ ID NO: 531 - DNA, 20 bp, Artificial
  Primer
ccagcatcaactgtcagagc

SEQ ID NO: 532 - DNA, 20 bp, Artificial
  Primer
ggcagattctttgccttctg

SEQ ID NO: 533 - DNA, 20 bp, Artificial
  Primer
attgcatgtagctcttgggg

SEQ ID NO: 534 - DNA, 20 bp, Artificial
  Primer
aagtgggtggcttcaacact

SEQ ID NO: 535 - DNA, 23 bp, Artificial
  Primer
agtggatcctgcatgttatgccg

SEQ ID NO: 536 - DNA, 22 bp, Artificial
  Primer
ccagacggacctttgtgggcaa

SEQ ID NO: 537 - DNA, 20 bp, Artificial
  Primer
aacagtggcaatggaagtgg

SEQ ID NO: 538 - DNA, 23 bp, Artificial
  Primer
agtcaggatctagtgggtgagtg

SEQ ID NO: 539 - DNA, 22 bp, Artificial
  Primer
cattgaacactgaaaggaaagc

SEQ ID NO: 540 - DNA, 20 bp, Artificial
  Primer
tcacaagggctttgaagtga

SEQ ID NO: 541 - DNA, 22 bp, Artificial
  Primer
gactaagcatatgaacctgggc

SEQ ID NO: 542 - DNA, 20 bp, Artificial
  Primer
ctgccccttgtcatctcatc

SEQ ID NO: 543 - DNA, 20 bp, Artificial
  Primer
tccctggacttcttgcagag

SEQ ID NO: 544 - DNA, 18 bp, Artificial
  Primer
ctggccccagacacaatc

SEQ ID NO: 545 - DNA, 20 bp, Artificial
  Primer
cactttgctgtggacctgaa

SEQ ID NO: 546 - DNA, 19 bp, Artificial
  Primer
acccaggaactgaacccat

SEQ ID NO: 547 - DNA, 21 bp, Artificial
  Primer
tttatcccaagaggttccacc

SEQ ID NO: 548 - DNA, 20 bp, Artificial
  Primer
aggtgcttgcagtgaatctg

SEQ ID NO: 549 - DNA, 20 bp, Artificial
  Primer
ccaccaggctaatgggtaaa

SEQ ID NO: 550 - DNA, 20 bp, Artificial
  Primer
tggtgttgcatctgcattct

SEQ ID NO: 551 - DNA, 20 bp, Artificial
  Primer
ctgggaagccttttgatctg

SEQ ID NO: 552 - DNA, 20 bp, Artificial
  Primer
atggaccagatggtggaaat

SEQ ID NO: 553 - DNA, 20 bp, Artificial
  Primer
ctttcccatcctttcaccaa

SEQ ID NO: 554 - DNA, 20 bp, Artificial
  Primer
aagtagggtgtgtgggggta

SEQ ID NO: 555 - DNA, 20 bp, Artificial
  Primer
gttgagcagggggtaacaag

SEQ ID NO: 556 - DNA, 20 bp, Artificial
  Primer
acgagtccctgctgctctac

SEQ ID NO: 557 - DNA, 24 bp, Artificial
  Primer
ccataactctgggacttttcctca

SEQ ID NO: 558 - DNA, 24 bp, Artificial
  Primer
atgttcagccatctctcctggtcc